(19) United States
(12) Patent Application Publication
(10) Pub. No.: US 2024/0352105 A1
(43) Pub. Date: Oct. 24, 2024

(54) COMPOSITIONS AND METHODS FOR NEUTRALIZING ANTIGENS

(71) Applicant: PACAGEN, INC, Cambridge, MA (US)

(72) Inventor: Evan ZHAO, Cambridge, MA (US)

(21) Appl. No.: 18/637,810

(22) Filed: Apr. 17, 2024

Related U.S. Application Data
(63) Continuation of application No. PCT/US2024/024742, filed on Apr. 16, 2024.
(60) Provisional application No. 63/496,508, filed on Apr. 17, 2023.

(51) Int. Cl. C07K 16/18; C07K 16/14; C07K 16/16

(52) U.S. Cl. CPC C07K 16/18; C07K 16/14; C07K 16/16; C07K 2317/35; C07K 2317/569; C07K 2317/76; C07K 2317/92

(57) ABSTRACT

Provided are compositions and methods for neutralizing allergens or for treatment of environmental allergies. The compositions and methods may reduce, minimize, or prevent an allergic response to an environmental allergen.

### CROSS-REFERENCE

This application is a continuation of International Patent Application No. PCT/US2024/024742, filed Apr. 16, 2024, which claims the benefit of U.S. Provisional Application No. 63/496,508, filed Apr. 17, 2023, which application is incorporated herein by reference.

### SEQUENCE LISTING

The instant application contains a Sequence Listing which has been submitted electronically in XML file format and is hereby incorporated by reference in its entirety. Said XML file, created on Apr. 2, 2024, is named 65491-701_601_SL.xml and is 70,130 bytes in size.

### BACKGROUND

Many people have animal, plant, or dust mite allergies, especially people having other allergies or asthma. Despite efforts to develop approaches that reduce, minimize, or prevent an allergic response to animal allergens, there remains an unmet need to overcome the inherent limitations of conventional methods at reasonable costs. The present disclosure addresses these needs and offers related advantages.

### SUMMARY

Provided herein is an allergen binding protein that binds or neutralizes an allergen, wherein the allergen binding protein comprises a nanobody, a monobody, a DARPin, or a small peptide below about 25 kilodalton (kDa) in molecular weight or under about 300 amino acids in length.

Also provided herein is an allergen binding protein that binds or neutralizes an allergen, wherein the allergen binding protein comprises a nanobody or small peptide having at least 80%, at least 85%, at least 90%, at least 95%, at least 97%, at least 99%, or 100% sequence identity to a sequence selected from the group consisting of SEQ ID NOs: 1-57 and 67-73.

In some embodiments, the allergen binding protein is formulated for coating a food or spraying, misting, or brushing an animal or household surface.

In some embodiments, the food is a pet food.

In some embodiments, the allergen is a pet allergen.

In some embodiments, the allergen binding protein is formulated to be applied over the food as a topper.

In some embodiments, the allergen binding protein is formulated to be mixed in with the food.

In some embodiments, the allergen binding protein is at a concentration of about 0.01 milligram per milliliter (mg/ml) to about 500 mg/ml in solution or after suspension.

In some embodiments, the allergen induces an allergic reaction in a human.

In some embodiments, the allergen comprises an environmental allergen.

In some embodiments, the allergen comprises an animal allergen.

In some embodiments, the allergen comprises a pet allergen.

In some embodiments, the allergen comprises a cat, a dog, a rabbit, a mouse, or a cockroach allergen.

In some embodiments, the allergen is selected from the group consisting of Fel d 1, Fel d 2, Fel d 3, Fel d 4, Can f 1, Can f 2, Can f 4, Can f 7, Ory C 1, Mus M 1, and Bla G 2.

In some embodiments, the allergen is Fel d 1.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 49-57.

In some embodiments, the allergen binding protein comprises a nanobody comprising an amino acid sequence having at least 80%, at least 85%, at least 90%, at least 95%, at least 97%, at least 99%, or 100% sequence identity to a sequence of SEQ ID NO: 49.

In some embodiments, the allergen binding protein comprises a nanobody having a sequence of SEQ ID NO: 49.

In some embodiments, the allergen binding protein comprises a nanobody comprising an amino acid sequence having at least 80%, at least 85%, at least 90%, at least 95%, at least 97%, at least 99%, or 100% sequence identity to a sequence of SEQ ID NO: 57.

In some embodiments, the allergen binding protein comprises a nanobody having a sequence of SEQ ID NO: 57.

In some embodiments, the allergen is Can f 1.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 67-73.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 67.

In some embodiments, the allergen binding protein comprises a nanobody having a sequence consisting of SEQ ID NO: 67.

In some embodiments, the allergen is Can f 2.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 9-23.

In some embodiments, wherein the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 16.

In some embodiments, the allergen binding protein comprises a nanobody comprising an amino acid sequence consisting of SEQ ID NO: 16.

In some embodiments, the allergen comprises a dust allergen.

In some embodiments, the dust allergen comprises Der p1 or Der p2.

In some embodiments, the dust allergen is Der p1.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 24-34.

In some embodiments, the allergen binding protein comprises a peptide having least 80%, at least 85%, at least 90%, at least 95%, at least 97%, at least 99%, or 100% sequence identity to a sequence selected from the group consisting of SEQ ID NOs: 35-48.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 27.

In some embodiments, the allergen binding protein comprises a nanobody having a sequence consisting of SEQ ID NO: 27.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 28.

In some embodiments, the allergen binding protein comprises a nanobody having a sequence consisting of SEQ ID NO: 28.

In some embodiments, the dust allergen is Der p2.

In some embodiments, the allergen binding protein comprises a nanobody having at least 80%, at least 85%, at least 90%, or 100% sequence identity to a sequence selected from the group consisting of SEQ ID NOs: 1-8.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 3.

In some embodiments, the allergen binding protein comprises a nanobody having a sequence consisting of SEQ ID NO: 3.

In some embodiments, the allergen comprises a plant or plant pollen allergen.

In some embodiments, the plant or pollen allergen is selected from the group consisting of Bet v1, Phl p 5, Phl p 1, Poa p 1, Cyn d 1, Bet v 2, Ole e 1, Amb a 1, Amb a 11, and Art v 1.

In some embodiments, the allergen comprises a mold allergen.

In some embodiments, the mold allergen is selected from the group consisting of Alt a 1, Asp f 1, Asp f 2, Cla h 8, Pen ch 13, and Pen ch 18.

In some embodiments, the allergen is a food allergen.

In some embodiments, the food allergen is selected from the group consisting of Pen a 1, Ara h 1, Ara h 3.

In some embodiments, the allergen binding protein comprises at least one modified amino acid.

In some embodiments, the allergen binding protein comprises about 1, about 5, about 10, about 15, about 20, about 25 or about 30 modified amino acids.

In some embodiments, the modified amino acid comprises a non-canonical amino acid.

In some embodiments, the non-canonical amino acid is selected from the group consisting of para-benzoylphenylalanine, 3,4-dyhydroxyphenylalanine, a tetrazine, a clooctene, homopropargylglycine, para-proparglyoxyphenylalanine, para-azidophenylalanine, para-isothiocynate phenylalanine, para-benzoylphenylalanine, para-cyanophenylalanine, para-nitrophenylalanine, a m-halogenated tyrosine analog, a halogenated proline analog, a halogenated tryptophan analog, and a halogenated leucine analog.

In some embodiments, the solubility of the allergen binding protein is increased by about 25%, 50%, 75%, 100%, 200%, 300%, 400%, 500%, 600% or more compared to an unmodified allergen binding protein.

In some embodiments, the binding affinity of the allergen binding protein is increased by about 25%, 50%, 75%, 100%, 200%, 300%, 400%, 500%, 600%, 700%, 800%, 900%, 1000%, 1500%, or more compared to an unmodified allergen binding protein.

In some embodiments, the thermal stability of the allergen binding protein is increased by about 10%, 25%, 50%, 75%, 100%, 200%, 300%, or more compared to an unmodified allergen binding protein.

In some embodiments, the allergen binding protein is a multivalent allergen binding protein.

In some embodiments, the multivalent allergen binding protein is a bivalent allergen binding protein.

In some embodiments, the binding affinity of the multivalent allergen binding protein allergen binding protein is increased by about 25%, 50%, 75%, 100%, 200%, 300%, 400%, 500%, 600%, 700%, 800%, 900%, 1000%, 1500%, or more compared to a monovalent allergen binding protein.

In some embodiments, the allergen binding protein further comprises a carrier.

In some embodiments, the carrier comprises a solvent, a diluent, a dispersion medium, or a coating.

In some embodiments, the carrier comprises silica.

In some embodiments, allergen binding protein is in a dry form.

In some embodiments, the carrier comprises water.

In some embodiments, the allergen binding protein comprises a liquid.

In some embodiments, the allergen binding protein further comprises a preservative.

In some embodiments, the preservative comprises potassium sorbate, EDTA, benzoic acid, phenoxyethanol, or maltol.

In some embodiments, the allergen binding protein further comprises a stabilizing agent or thickener.

In some embodiments, the stabilizing agent or thickener comprises dextrin, maltodextrin, glycerol, glucose, sucrose, or trehalose.

In some embodiments, the allergen binding protein further comprises an isotonic agent.

In some embodiments, the allergen binding protein is suspended or resuspended in a solvent.

In some embodiments, the solvent comprises water.

Also provided herein is a composition, comprising: an allergen binding protein that binds or neutralizes an allergen, wherein the allergen binding protein comprises a nanobody, a monobody, a DARPin, or a small peptide below about 25 kilodalton in molecular weight or under about 300 amino acids in length.

Also provided herein is a composition, comprising: an allergen binding protein that binds or neutralizes an allergen, wherein the allergen binding protein comprises a nanobody or a small peptide below about 25 kilodalton in molecular weight or under about 300 amino acids in length, wherein the allergen binding protein comprises a sequence having at least 80%, at least 85%, at least 90%, at least 95%, at least 97%, at least 99%, or 100% sequence identity to a sequence selected from the group consisting of SEQ ID NOs: 1-57 and 67-73.

In some embodiments, the composition is formulated for coating a food or spraying, misting, or brushing an animal or household surface.

In some embodiments, the food is a pet food.

In some embodiments, the allergen is a pet allergen.

In some embodiments, the composition is formulated to be applied over the food as a topper.

In some embodiments, the composition is formulated to be mixed in with the food.

In some embodiments, the allergen binding protein is at a concentration of about 0.01 milligram per milliliter (mg/ml) to about 500 mg/ml in solution or after suspension.

In some embodiments, the allergen induces an allergic reaction in a human.

In some embodiments, the allergen comprises an environmental allergen.

In some embodiments, the allergen comprises an animal allergen.

In some embodiments, the allergen comprises a pet allergen.

In some embodiments, the allergen comprises a cat, a dog, a rabbit, a mouse, or a cockroach allergen.

In some embodiments, the allergen is selected from the group consisting of Fel d 1, Fel d 2, Fel d 3, Fel d 4, Can f 1, Can f 2, Can f4, Can f 7, Ory C 1, Mus M 1, and Bla G 2. In some embodiments, the allergen is Fel d 1.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 49-57.

In some embodiments, the allergen binding protein comprises a nanobody comprising an amino acid sequence having at least 80%, at least 85%, at least 90%, at least 95%, at least 97%, at least 99%, or 100% sequence identity to a sequence of SEQ ID NO: 49.

In some embodiments, the allergen binding protein comprises a nanobody having a sequence of SEQ ID NO: 49.

In some embodiments, the allergen binding protein comprises a nanobody comprising an amino acid sequence having at least 80%, at least 85%, at least 90%, at least 95%, at least 97%, at least 99%, or 100% sequence identity to a sequence of SEQ ID NO: 57.

In some embodiments, the allergen binding protein comprises a nanobody having a sequence of SEQ ID NO: 57.

In some embodiments, the allergen is Can f 1.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 67-73.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 67.

In some embodiments, the allergen binding protein comprises a nanobody having a sequence consisting of SEQ ID NO: 67.

In some embodiments, the allergen is Can f 2.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 9-23.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 16.

In some embodiments, the allergen binding protein comprises a nanobody comprising an amino acid sequence consisting of SEQ ID NO: 16.

In some embodiments, the allergen comprises a dust allergen.

In some embodiments, the dust allergen comprises Der p1 or Der p2.

In some embodiments, the dust allergen is Der p1.

In some embodiments, the allergen binding protein comprises a nanobody comprising an amino acid sequence having at least 80%, at least 85%, at least 90%, at least 95%, at least 97%, at least 99%, or 100% sequence identity to a sequence selected from the group consisting of SEQ ID NOs: 24-34.

In some embodiments, the allergen binding protein comprises a peptide having least 80%, at least 85%, at least 90%, at least 95%, at least 97%, at least 99%, or 100% sequence identity to a sequence selected from the group consisting of SEQ ID NOs: 35-48.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 27.

In some embodiments, the allergen binding protein comprises a nanobody having a sequence consisting of SEQ ID NO: 27.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 28.

In some embodiments, the allergen binding protein comprises a nanobody having a sequence consisting of SEQ ID NO: 28.

In some embodiments, the dust allergen is Der p2.

In some embodiments, the allergen binding protein comprises a nanobody having at least 80%, at least 85%, at least 90%, or 100% sequence identity to a sequence selected from the group consisting of SEQ ID NOs: 1-8.

In some embodiments, the allergen binding protein comprises a nanobody comprising an amino acid sequence having at least 80%, at least 85%, at least 90%, at least 95%, at least 97%, at least 99%, or 100% sequence identity to a sequence set forth in SEQ ID NO: 3.

In some embodiments, the allergen binding protein comprises a nanobody having a sequence consisting of SEQ ID NO: 3.

In some embodiments, the allergen comprises a plant or plant pollen allergen.

In some embodiments, the plant or pollen allergen is selected from the group consisting of Bet v1, Phl p 5, Phl p 1, Poa p 1, Cyn d 1, Bet v 2, Ole e 1, Amb a 1, Amb a 11, and Art v 1.

In some embodiments, the allergen comprises a mold allergen.

In some embodiments, the mold allergen is selected from the group consisting of Alt a 1, Asp f 1, Asp f 2, Cla h 8, Pen ch 13, and Pen ch 18.

In some embodiments, the allergen is a food allergen.

In some embodiments, the food allergen is selected from the group consisting of Pen a 1, Ara h 1, Ara h 3.

In some embodiments, the allergen binding protein comprises at least one amino acid modification.

In some embodiments, the allergen binding protein comprises about 1, about 5, about 10, about 15, about 20, about 25 or about 30 amino acid modifications.

In some embodiments, the amino acid modification comprises introduction of a non-canonical amino acid.

In some embodiments, the non-canonical amino acid is selected from the group consisting of para-benzoylphenylalanine, 3,4-dyhydroxyphenylalanine, a tetrazine, clooctenes, homopropargylglycine, para-proparglyoxyphenylalanine, para-azidophenylalanine, para-isothiocynate phenylalanine, para-benzoylphenylalanine, para-cyanophenylalanine, para-nitrophenylalanine, a m-halogenated tyrosine analog, a halogenated proline analog, a halogenated tryptophan analog, and a halogenated leucine analog.

In some embodiments, the solubility of the composition is increased by about 25%, 50%, 75%, 100%, 200%, 300%, 400%, 500%, 600% or more compared to an unmodified allergen binding protein.

In some embodiments, the binding affinity of the allergen binding protein is increased by about 25%, 50%, 75%, 100%, 200%, 300%, 400%, 500%, 600%, 700%, 800%, 900%, 1000%, 1500%, or more compared to an unmodified allergen binding protein.

In some embodiments, the thermal stability of the allergen binding protein is increased by about 10%, 25%, 50%, 75%, 100%, 200%, 300%, or more compared to an unmodified allergen binding protein.

In some embodiments, the allergen binding protein is a multivalent allergen binding protein.

In some embodiments, the multivalent allergen binding protein is a bivalent allergen binding protein.

In some embodiments, the binding affinity of the multivalent allergen binding protein composition is increased by about 25%, 50%, 75%, 100%, 200%, 300%, 400%, 500%, 600%, 700%, 800%, 900%, 1000%, 1500%, or more compared to a monovalent allergen binding protein.

In some embodiments, the composition further comprises a carrier.

In some embodiments, the carrier comprises a solvent, a diluent, a dispersion medium, or a coating.

In some embodiments, the carrier comprises silica.

In some embodiments, the composition is in a dry form.

In some embodiments, the carrier comprises water.

In some embodiments, the composition comprises a liquid.

In some embodiments, the composition further comprises a preservative.

In some embodiments, the preservative comprises potassium sorbate, EDTA, benzoic acid, phenoxyethanol, or maltol.

In some embodiments, the composition further comprises a stabilizing agent or thickener.

In some embodiments, the stabilizing agent or thickener comprises dextrin, maltodextrin, glycerol, glucose, sucrose, or trehalose.

In some embodiments, the composition further comprises an isotonic agent.

In some embodiments, the composition is suspended or resuspended in a solvent.

In some embodiments, the solvent comprises water.

Also provided herein is a method, comprising: spraying or misting the allergen binding protein of any one of the foregoing embodiments or the composition of any one of the foregoing embodiments, or contacting the surface with the composition of any one of the foregoing embodiments.

In some embodiments, the contacting comprises coating or brushing.

In some embodiments, the surface comprises a surface of an air filter or a humidifier.

In some embodiments, the surface comprises the allergen.

In some embodiments, the composition neutralizes the allergen on the surface.

In some embodiments, the surface comprises food.

In some embodiments, the food is consumed by an animal that is the source of the antigen.

In some embodiments, the surface comprises a pet accessory (e.g. collar or brush) or an area/product where an animal can sit or walk by (e.g. a dog bed).

In some embodiments, the misting is performed by a humidifier or a spray bottle.

In some embodiments, about 0.01 milliliter (ml) to about 20 ml of the composition sprayed per about 1 square meter of area neutralizes the allergenicity of the antigen.

Also provided herein is a method of preparing the allergen binding protein of any one of the foregoing embodiments or the composition of any one of the foregoing embodiments, comprising: harvesting the allergen binding protein from an engineered microbe or from a secretion by the microbe, wherein the microbe comprises a heterologous nucleic acid encoding the allergen binding protein.

In some embodiments, the method further comprises incorporating the heterologous nucleic acid encoding the allergen binding protein into a cell-free protein expression system.

In some embodiments, the microbe comprises a yeast or bacterium.

In some embodiments, the microbe is a bacterium and comprises E. coli.

In some embodiments, the microbe is a yeast and comprises Pichia pastoris.

In some embodiments, the allergen binding protein is purified or concentrated from a secretion by the microbe.

In some embodiments, purification or concentration comprises a filtration step.

In some embodiments, the filtration step comprises passing the secretion through a filter of about 0.01 nm, 0.1 nm, 1 nm, 5 nm or more in size.

In some embodiments, the method does not comprise a centrifugation step.

Also provided herein is a method of treating an allergy in a subject in need thereof, the method comprising administering to the subject the allergen binding protein of any one of the foregoing embodiments or the composition of any one of the foregoing embodiments.

Also provided herein is method of treating an allergy in a subject in need thereof, the method comprising (i) administering to the subject a therapeutically effective amount of a microbe engineered to produce the allergen binding protein of any one of the foregoing embodiments or the composition of any one of the foregoing embodiments.

In some embodiments, the microbe is a yeast or bacterium.

In some embodiments, the microbe is a bacterium and comprises E. coli.

In some embodiments, the microbe is a yeast and comprises Pichia pastoris.

In some embodiments, the microbe is administered to the subject orally.

In some embodiments, the microbe produces the allergen binding protein or composition when ingested.

Also provided herein is a method of neutralizing an allergen, the method comprising (i) aerosolizing the allergen binding protein of any one of the foregoing embodiments or the composition of any one of the foregoing embodiments into a mist and (ii) contacting the mist to a surface comprising an allergen.

In some embodiments, the aerosolizing is performed by an aerosolization machine.

In some embodiments, the aerosolization machine is worn by a subject.

In some embodiments, the surface comprises a pet accessory or an area/product where an animal can sit or walk by.

Also provided herein is a method of neutralizing an allergen, the method comprising (i) aerosolizing the allergen binding protein of any one of the foregoing embodiments or the composition of any one of the foregoing embodiments into a mist and (ii) contacting the mist to a food.

In some embodiments, the food is a pet food.

In some embodiments, the allergen is a pet allergen.

In some embodiments, the mist is applied over the food as a topper.

In some embodiments, the mist is mixed into the food.

Also provided herein is a method of treating an allergy in a subject in need thereof, the method comprising aerosolizing the allergen binding protein of any one of the foregoing embodiments or the composition of any one of the foregoing embodiments into a mist.

In some embodiments, the aerosolizing is performed by an aerosolization machine.

In some embodiments, the aerosolization machine is worn.

In some embodiments, the aerosolization machine is worn by the subject.

In some embodiments, the method further comprises inhalation of the mist by the subject.

In some embodiments, the subject is a human.

In some embodiments, the subject is a non-human animal.

Also provided herein is a pharmaceutical composition comprising (i) the allergen binding protein of any one of the foregoing embodiments or the composition of any one of the foregoing embodiments and (ii) a pharmaceutically acceptable excipient.

Also provided herein is an allergen binding protein comprising a sequence of any one of SEQ ID NOs: 1-57 or 67-73.

Also provided herein is an allergen binding protein comprising a sequence of SEQ ID NO: 3.

Also provided herein is an allergen binding protein comprising a sequence of SEQ ID NO: 16.

Also provided herein is an allergen binding protein comprising a sequence of SEQ ID NO: 27.

Also provided herein is an allergen binding protein comprising a sequence of SEQ ID NO: 28.

Also provided herein is an allergen binding protein comprising a sequence of SEQ ID NO: 49.

Also provided herein is an allergen binding protein comprising a sequence of SEQ ID NO: 57.

Also provided herein is an allergen binding protein comprising a sequence of SEQ ID NO: 67.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a schematically illustration of a functional assay, in accordance with one or more embodiments of the present disclosure.

FIG. 2 depicts an SDS-PAGE Western Blot image of a recombinant His-tagged Der p2 protein, in accordance with one or more embodiments of the present disclosure.

FIG. 3A depicts results from a Der p2 yeast display VHH library depicting the sequence alignment of 50 randomly selected clones, in accordance with one or more embodiments of the present disclosure.

FIGS. 3B-3I are binding curves obtained from ELISA assays performed using the identified anti-Der p2 VHH clones, in accordance with one or more embodiments of the present disclosure.

FIG. 4 depicts an SDS-PAGE Western Blot image of a recombinant His-tagged Can f1 protein, in accordance with one or more embodiments of the present disclosure.

FIG. 5A depicts results from a Can fl yeast display VHH library depicting the sequence alignment of 50 randomly selected clones, in accordance with one or more embodiments of the present disclosure.

FIGS. 5B-5H are binding curves obtained from ELISA assays performed using the identified anti-Can f1 VHH clones, in accordance with one or more embodiments of the present disclosure.

FIG. 6 depicts an SDS-PAGE Western Blot image of a recombinant His-tagged Can f2 protein, in accordance with one or more embodiments of the present disclosure.

FIG. 7A depicts results from a Can f2 yeast display VHH library depicting the sequence alignment of 50 randomly selected clones, in accordance with one or more embodiments of the present disclosure.

FIGS. 7B-7P are binding curves obtained from ELISA assays performed using the identified anti-Can f2 VHH clones, in accordance with one or more embodiments of the present disclosure.

FIG. 8 depicts an SDS-PAGE Western Blot image of a recombinant His-tagged Der p1 protein, in accordance with one or more embodiments of the present disclosure.

FIGS. 9A-9K are binding curves obtained from ELISA assays performed using the identified anti-Der p2 VHH clones, in accordance with one or more embodiments of the present disclosure.

FIG. 10 depicts results of a Der p2 activity assay when Der p2 is incubated with the indicated anti-Der p2 VHH clones, in accordance with one or more embodiments of the present disclosure.

FIGS. 11A-11H depict results of Der p2 activity assays when Der p2 at varying concentrations is incubated with the indicated anti-Der p2 VHH clones, in accordance with one or more embodiments of the present disclosure.

FIG. 12 depicts results from a Der p1 phage display 7-amino acid peptide library depicting the sequence alignment of resulting peptides, in accordance with one or more embodiments of the present disclosure.

FIG. 13 is a binding curve obtained from an ELISA assay performed using the identified peptide clone 2-D3, in accordance with one or more embodiments of the present disclosure.

FIG. 14 depicts an SDS-PAGE Western Blot image of a recombinant His-tagged Fel d1 protein, in accordance with one or more embodiments of the present disclosure.

FIG. 15A depicts results from a Fel d1 yeast display VHH library depicting the sequence alignment of 50 randomly selected clones, in accordance with one or more embodiments of the present disclosure.

FIGS. 15B-15D are binding curves obtained from ELISA assays performed using the identified anti-Fel d1 VHH clones specific for Fel d1 epitope ENARILKNCVDAKM (SEQ ID NO: 61), in accordance with one or more embodiments of the present disclosure.

FIG. 16 is a binding curve obtained from an ELISA assay performed using the identified anti-Fel d1 VHH clone specific for Fel d1 epitope FAVANGNELLLDLS (SEQ ID NO: 59), in accordance with one or more embodiments of the present disclosure.

FIGS. 17A-17E are binding curves obtained from ELISA assays performed using the identified anti-Fel d1 VHH clones specific for Fel d1 epitope AKMTEEDKENALS (SEQ ID NO: 60), in accordance with one or more embodiments of the present disclosure.

FIG. 18 is a binding curve obtained from an ELISA assay performed using the identified anti-Fel d1 VHH clone specific for Fel d1 epitope VAQYKALPVVLENA (SEQ ID NO: 58).

FIGS. 19A-19I depict results from a thermostability assay performed using the identified anti-Fel d1 VHH clones, in accordance with one or more embodiments of the present disclosure.

FIGS. 20A-20C depict size exclusion chromatography (SEC) graphs obtained testing the neutralizing ability of anti-Fel d1 VHH clone C3, in accordance with one or more embodiments of the present disclosure.

### DETAILED DESCRIPTION

An allergy may include an abnormal response from the immune system to exposure to an allergen. The human body's natural immune system may create antibodies. Antibodies may include Y-shaped proteins that bind to different foreign proteins or chemicals interacting with them. An allergic reaction may occur when the immune system reacts strangely with a foreign substance (allergen) and enters a hyperactive state of defense (immune cell TH2 response, etc.). This defense can sometimes be set off by immunoglobulin E antibodies (IgE) that mast cells use to sense foreign substances, in this case the allergen. When people inhale or come into contact with an allergen, the immune system may react, causing an inflammatory response in the nasal passages or lungs. Prolonged exposure to allergens may cause persistent inflammation associated with asthma.

About 7 out of 10 households in the United States have a pet, and approximately 15 to 30 percent of the population is allergic to pets, specifically dogs or cats. Cat and dog allergens may be found in the animal's shed skin cells (dander), saliva, urine, sweat, and fur. Dander may cause a problem because it is very small and may remain airborne. Cats may be one of the major sources of indoor inhalant allergens. The global incidence of cat allergies is rapidly increasing and has been considered a major public health problem. Some examples of cat allergens may include Fel d 1 to Feld d 8. More than about 95% of cat allergies may be caused by a secretoglobin, called Fel d 1. Cats may secrete the Fel d1 protein from their salivary and sebaceous glands, and spread it throughout their fur during regular tongue baths. Fel d 1 may be easily airborne and remain in the indoor environment. Canis familiaris allergen 1 (Can f1) and Canis familiaris allergen 1 (Can f2) may be the two allergens present in dog hair or dander extracts. Dog allergens may also present in dander, saliva, urine, and blood. Allergen levels may differ among breeds and all breeds may trigger allergies including even hairless dogs. There are the Group I (e.g., Der p I and Der f I) and Group II (e.g., Der p II and Der f II) protein allergens in house dust mite allergy.

A number of other allergens can also induce allergic reactions. Examples of pet allergens include, but are not limited to Fel d 1, Fel d 2, Fel d 3, Fel d 4, Can f 1, Can f 2, Can f 4, Can f 7, Ory C 1, Mus M 1, or Bla G 2. Examples of plant or pollen allergens include, but are not limited to Bet v1, Phl p 5, Phl p 1, Poa p 1, Cyn d 1, Bet v 2, Ole e 1, Amb a 1, Amb a 11, and Art v 1. Examples of mold allergens include, but are not limited to Alt a 1, Asp f 1, Asp f 2, Cla h 8, Pen ch 13, and Pen ch 18. Examples of food allergens include, but are not limited to Pen a 1, Ara h 1, Ara h 3.

The human body may produce antibodies that bind to the allergen and negate the allergic reaction. Antibodies may be produced by cells outside of the human body and used to neutralize allergens. However, antibody production may be expensive because it is produced using human, mammalian, or avian cells. For example, a conventional approach may involve immunizing eggs/chickens with Fel d 1 and feeding these eggs to the cats to reduce the cost of production. This approach may temporarily neutralize Fel d 1 in the cat's mouth and the results are not perfect.

Alpacas, llamas, and camels may produce similar immune proteins. Scientists can isolate a small section of the immune proteins, called a single-domain antibody (sdAb) or nanobody. The utility and advantages of single-domain antibodies or nanobodies may include but are not limited to, their smaller size, larger number of accessible epitopes, relatively low production costs due to production in bacteria or yeast and improved robustness, as compared with their full-length antibodies. These also may be produced from bacteria or yeast at very low cost. Peptides with short amino acid chains may also be designed to bind allergens. There are other small protein binders that can be designed to bind and neutralize allergens such as monobodies and designed ankyrin repeat proteins (DARPins).

### Allergen Binding Proteins

In certain aspects, the present disclosure provides an allergen binding protein. In some embodiments, the allergen binding protein may bind or neutralize the allergen. In some embodiments, the allergen binding protein comprises an antibody fragment. The antibody fragment may be monoclonal. The antibody fragment may be polyclonal. In some embodiments, the allergen binding protein comprises a nanobody, a monobody, a DARPin, or a small peptide. In some embodiments, the allergen binding protein may be synthetic. In some embodiments, the allergen binding protein may be engineered. In some embodiments, the allergen binding protein may be recombinant.

In some embodiments, the present disclosure provides an allergen binding protein that binds or neutralizes the allergen. In some embodiments, the allergen binding protein comprises a nanobody, a monobody, a DARPin, or a small peptide below about 25 kilodalton (kDa) in molecular weight or under about 300 amino acids in length. In some embodiments, the allergen binding protein may be formulated for coating a food or spraying, misting, or brushing a surface of an animal or a household. In some embodiments, the food is a pet food. In some embodiments, the allergen is a pet allergen. In some embodiments, the allergen binding protein is formulated to be applied over the food as a topper. In some embodiments, the allergen binding protein is formulated to be mixed in with the food.

In some embodiments, the allergen binding protein may be a nanobody. In some embodiments, the allergen binding protein may be a monobody. In some embodiments, the allergen binding protein may be a DARPin. In some embodiments, DARPins are small, single domain proteins of about 14 kDa which can be selected to bind any given target protein with high affinity and specificity. In some embodiments, the allergen binding protein may be a small peptide.

In some embodiments, the allergen binding protein described herein may be a nanobody. In some embodiments, the allergen binding protein may be a single-domain antibody. The utility and advantages of single-domain antibodies (sdAbs), may include but are not limited to, their smaller size, larger number of accessible epitopes, relatively low production costs, or improved robustness, as compared with their full-length antibodies.

In some embodiments, the allergen binding protein comprises a nanobody or peptide having at least 60%, at least 70%, at least 80%, at least 85%, at least 90%, at least 95%, at least 97%, at least 99%, or 100% sequence identity to a sequence selected from the group consisting of SEQ ID NOs: 1-57 and 67-73. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 1. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 2. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 3. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 4. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 5. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 6. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 7. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 8. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 9. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 10. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 11. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 12. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 13. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 14. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 15. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 16. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 17. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 18. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 19. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 20. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 21. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 22. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 23. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 24. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 25. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 26. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 27. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 28. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 29. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 30. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 31. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 32. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 33. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 34. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 49. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 50. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 51. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 52. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 53. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 54. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 55. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 56. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 57. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 67. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 68. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 69. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 70. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 71. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 72. In some embodiments, the allergen binding protein comprises a nanobody having at least 60%, at least 70%, at least 80%, at least 85%, at least 90%, at least 95%, at least 97%, at least 99%, or 100% sequence identity to a sequence of SEQ ID NO: 73.

In some embodiments, the allergen binding protein comprises a nanobody having a sequence of SEQ ID NO: 1. In some embodiments, the allergen binding protein comprises a nanobody having a sequence of SEQ ID NO: 2. In some embodiments, the allergen binding protein comprises a nanobody having a sequence of SEQ ID NO: 3. In some embodiments, the allergen binding protein comprises a nanobody having a sequence of SEQ ID NO: 4. In some embodiments, the allergen binding protein comprises a nanobody having a sequence of SEQ ID NO: 5. In some embodiments, the allergen binding protein comprises a nanobody having a sequence of SEQ ID NO: 6. In some embodiments, the allergen binding protein comprises a nanobody having a sequence of SEQ ID NO: 7. In some embodiments, the allergen binding protein comprises a nanobody having a sequence of SEQ ID NO: 8. In some embodiments, the allergen binding protein comprises a nanobody having a sequence of SEQ ID NO: 9. In some embodiments, the allergen binding protein comprises a nanobody having a sequence of SEQ ID NO: 10. In some embodiments, the allergen binding protein comprises a nanobody having a sequence of SEQ ID NO: 11. In some embodiments, the allergen binding protein comprises a nanobody having a sequence of SEQ ID NO: 12. In some embodiments, the allergen binding protein comprises a nanobody having a sequence of SEQ ID NO: 13. In some embodiments, the allergen binding protein comprises a nanobody having a sequence of SEQ ID NO: 14. In some embodiments, the allergen binding protein comprises a nanobody having a sequence of SEQ ID NO: 15. In some embodiments, the allergen binding protein comprises a nanobody having a sequence of SEQ ID NO: 16. In some embodiments, the allergen binding protein comprises a nanobody having a sequence of SEQ ID NO: 17. In some embodiments, the allergen binding protein comprises a nanobody having a sequence of SEQ ID NO: 18. In some embodiments, the allergen binding protein comprises a nanobody having a sequence of SEQ ID NO: 19. In some embodiments, the allergen binding protein comprises a nanobody having a sequence of SEQ ID NO: 20. In some embodiments, the allergen binding protein comprises a nanobody having a sequence of SEQ ID NO: 21. In some embodiments, the allergen binding protein comprises a nanobody having a sequence of SEQ ID NO: 22. In some embodiments, the allergen binding protein comprises a nanobody having a sequence of SEQ ID NO: 23. In some embodiments, the allergen binding protein comprises a nanobody having a sequence of SEQ ID NO: 24. In some embodiments, the allergen binding protein comprises a nanobody having a sequence of SEQ ID NO: 25. In some embodiments, the allergen binding protein comprises a nanobody having a sequence of SEQ ID NO: 26. In some embodiments, the allergen binding protein comprises a nanobody having a sequence of SEQ ID NO: 27. In some embodiments, the allergen binding protein comprises a nanobody having a sequence of SEQ ID NO: 28. In some embodiments, the allergen binding protein comprises a nanobody having a sequence of SEQ ID NO: 29. In some embodiments, the allergen binding protein comprises a nanobody having a sequence of SEQ ID NO: 30. In some embodiments, the allergen binding protein comprises a nanobody having a sequence of SEQ ID NO: 31. In some embodiments, the allergen binding protein comprises a nanobody having a sequence of SEQ ID NO: 32. In some embodiments, the allergen binding protein comprises a nanobody having a sequence of SEQ ID NO: 33. In some embodiments, the allergen binding protein comprises a nanobody having a sequence of SEQ ID NO: 34. In some embodiments, the allergen binding protein comprises a nanobody having a sequence of SEQ ID NO: 49. In some embodiments, the allergen binding protein comprises a nanobody having a sequence of SEQ ID NO: 50. In some embodiments, the allergen binding protein comprises a nanobody having a sequence of SEQ ID NO: 51. In some embodiments, the allergen binding protein comprises a nanobody having a sequence of SEQ ID NO: 52. In some embodiments, the allergen binding protein comprises a nanobody having a sequence of SEQ ID NO: 53. In some embodiments, the allergen binding protein comprises a nanobody having a sequence of SEQ ID NO: 54. In some embodiments, the allergen binding protein comprises a nanobody having a sequence of SEQ ID NO: 55. In some embodiments, the allergen binding protein comprises a nanobody having a sequence of SEQ ID NO: 56. In some embodiments, the allergen binding protein comprises a nanobody having a sequence of SEQ ID NO: 57. In some embodiments, the allergen binding protein comprises a nanobody having a sequence of SEQ ID NO: 67. In some embodiments, the allergen binding protein comprises a nanobody having a sequence of SEQ ID NO: 68. In some embodiments, the allergen binding protein comprises a nanobody having a sequence of SEQ ID NO: 69. In some embodiments, the allergen binding protein comprises a nanobody having a sequence of SEQ ID NO: 70. In some embodiments, the allergen binding protein comprises a nanobody having a sequence of SEQ ID NO: 71. In some embodiments, the allergen binding protein comprises a nanobody having a sequence of SEQ ID NO: 72. In some embodiments, the allergen binding protein comprises a nanobody having a sequence of SEQ ID NO: 73.

In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 35. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 36. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 37. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 38. In some embodiments, the allergen binding protein comprises a peptide having at least 60%, at least 70%, at least 80%, at least 85%, at least 90%, at least 95%, at least 97%, at least 99%, or 100%-sequence identity to SEQ ID NO: 39. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 40. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 41. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 42. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 43. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 44. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 45. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 46. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 47. In some embodiments, the allergen binding protein comprises a peptide having at least 60%, at least 70%, at least 80%, at least 85%, at least 90%, at least 95%, at least 97%, at least 99%, or 100% sequence identity to SEQ ID NO: 48.

In some embodiments, the allergen binding protein comprises a peptide having a sequence of SEQ ID NO: 35. In some embodiments, the allergen binding protein comprises a peptide having a sequence of SEQ ID NO: 36. In some embodiments, the allergen binding protein comprises a peptide having a sequence of SEQ ID NO: 37. In some embodiments, the allergen binding protein comprises a peptide having a sequence of SEQ ID NO: 38. In some embodiments, the allergen binding protein comprises a peptide having a sequence of SEQ ID NO: 39. In some embodiments, the allergen binding protein comprises a peptide having a sequence of SEQ ID NO: 40. In some embodiments, the allergen binding protein comprises a peptide having a sequence of SEQ ID NO: 41. In some embodiments, the allergen binding protein comprises a peptide having a sequence of SEQ ID NO: 42. In some embodiments, the allergen binding protein comprises a peptide having a sequence of SEQ ID NO: 43. In some embodiments, the allergen binding protein comprises a peptide having a sequence of SEQ ID NO: 44. In some embodiments, the allergen binding protein comprises a peptide having a sequence of SEQ ID NO: 45. In some embodiments, the allergen binding protein comprises a peptide having a sequence of SEQ ID NO: 46. In some embodiments, the allergen binding protein comprises a peptide having a sequence of SEQ ID NO: 47. In some embodiments, the allergen binding protein comprises a peptide having a sequence of SEQ ID NO: 48.

In some embodiments, the allergen binding protein described herein may be a humanized antibody fragment, a nanobody, a variant, or a derivative thereof, that may, for example, be formulated for administration to a human. In some embodiments, the humanized antibody may be chimeric humanized antibody or fully human antibody, for example, comprising an amino acid sequence from or with similarity to a human antibody amino acid sequence, and a non-human amino acid sequence. For example, a portion of the heavy and/or light chain of a chimeric humanized antibody may be identical to or similar to a corresponding sequence in a human antibody, while the remainder of the chain(s) may be non-human, for example, identical or similar to a corresponding sequence in an antibody derived from another species, or belonging to another antibody class or subclass. The non-human sequence may be humanized to reduce the likelihood of immunogenicity while preserving target specificity, for example, by incorporation of human DNA to the genetic sequence of the genes that produce the antibodies in the non-human animal. Humanized antibody may be fully human antibody, for example, containing an amino acid sequence that is a human antibody amino acid sequence.

In some embodiments, the allergen binding protein described herein comprises a signal peptidase or peptide protein fusion. Signal peptides may result in higher protein expression and/or secretion by a cell. Signal peptidases may cleave a signal peptide off the antibody or the antigen-binding fragment thereof, for example, during a secretion process, generating a mature antibody that does not comprise the signal peptide sequence.

The constant regions of the allergen binding protein described herein may mediate various effector functions, and may be minimally involved in antigen binding.

The allergen binding protein described herein comprises constant regions that are selected or modified to provide suitable protein characteristics, for example, suitable characteristics for treating a disease or condition as disclosed herein.

The variable (V) regions may mediate antigen binding and define the specificity of a particular antibody for an antigen.

Within hypervariable regions are amino acid residues that primarily determine the binding specificity of the antibody. Sequences comprising these residues are known as complementarity determining regions (CDRs). One antigen binding site of an allergen binding protein can be comprised of several variable loops that confer specificity to the allergen binding protein.

In some embodiments, the allergen binding protein described herein comprises variants or derivatives thereof. For example, a non-human animal, bacteria, yeast, or plant may be genetically modified to produce protein variants or derivatives. In some embodiments, an allergen binding protein may be a single-domain antibody (sdAb), for example, a heavy chain only antibody (HCAb) VHH, nanobody, monobody, DARPin, small peptide or scFV.

In other embodiments, the allergen binding protein described herein may be a binding fragment thereof. In some cases, the allergen binding protein described herein may be a humanized antibody or binding fragment thereof, a chimeric antibody or binding fragment thereof, a monoclonal antibody or binding fragment thereof, a multi-specific antibody or binding fragment thereof, a bispecific antibody or binding fragment thereof, or a single-domain antibody (e.g. Nanobody®) thereof. In some cases, the allergen binding protein described herein may be monovalent Fab′, divalent Fab2, F(ab)′3 fragments, single-chain variable fragment (scFv), bis-scFv, (scFv) 2, diabody, minibody, nanobody, triabody, tetrabody, disulfide stabilized Fv protein (“dsFv”), single-domain antibody (sdAb), Ig NAR, camelid antibody or binding fragment thereof, or a chemically modified derivative thereof.

In some embodiments, the allergen binding protein described herein may be a multi-specific antibody. In some cases, the multi-specific protein comprises two or more target binding moieties in which each of the two or more target binding moieties binds specifically to an antigen, and the two or more antigens are different. In some cases, the multi-specific antibody comprises target binding moieties that specifically bind to three or more different antigens, four or more different antigens, or five or more different antigens. In some embodiments, the antibody may be a bispecific antibody. In some cases, the bispecific antibody or binding fragment includes, but is not limited to a Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), FcAAdp, XmAb, Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), or nanobody. In some embodiments, the bispecific antibody may be a trifunctional antibody or a bispecific mini-antibody. In some cases, the bispecific antibody may be a trifunctional antibody. The trifunctional antibody may be a full length monoclonal antibody comprising binding sites for two different antigens.

In some embodiments, the allergen binding protein described herein comprises one or more mutations to stabilize the protein and/or to increase half-life.

In some embodiments, the allergen binding protein described herein comprises a humanized antibody or binding fragment thereof or a chimeric antibody or binding fragment thereof. In some embodiments, the comprises a multi-specific antibody or binding fragment thereof. In some embodiments, the antibody comprises a bispecific antibody or binding fragment thereof. In some embodiments, the antibody may be an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, F(ab′)2, F(ab′)2-scFv2. scFv-KIH, Fab-scFv-Fc, tetravalent HCAb, scDiabody-Fc, diabody-Fc, tandem scFv-Fc, or intrabody. In some cases, the antibody is monovalent Fab′, divalent Fab2, F(ab)′3 fragments, single-chain variable fragment (scFv), bis-scFv, (scFv) 2, diabody, minibody, nanobody, triabody, tetrabody, disulfide stabilized Fv protein (“dsFv”), single-domain antibody (e.g., a nanobody), Ig NAR, camelid antibody, or binding fragment thereof, or a chemically modified derivative thereof.

In some embodiments, the allergen binding protein described herein (e.g., single-domain antibody) may bind to an epitope expressed by the target cell associated with the disease or condition described herein. In some embodiments, the antibody or the antigen-binding fragment thereof may bind to an epitope associated with the microenvironment described herein.

In some embodiments, the exogenous protein may function as an agonist or an antagonist, where upon binding to any one of the epitope described herein, the binding of the antibody or the single-domain antibody induces agonist or antagonist effect.

In some embodiments, the antigen binding protein may be about 1, about 2, about 3, about 4, about 5, about 6, about 7, about 8, about 9, or about 10 nm in length. In some embodiments, the antigen binding protein may be at most 1, at most 2, at most 3, at most 4, at most 5, at most 6, at most 7, at most 8, at most 9, or at most 10 nm in length. In some embodiments, the antigen binding protein may be at least 1, at least 2, at least 3, at least 4, at least 5, at least 6, at least 7, at least 8, at least 9, or at least 10 nm in length. In some embodiments, the antigen binding protein may be between two values described above, for example between about 1 to about 10, about 2 to about 9, about 3 to about 8, about 4 to about 7, or about 5 to about 6 nm in length. In some embodiments, the antigen binding protein may be between two values described above, for example between 1 to 10, 2 to 9, 3 to 8, 4 to 7, or 5 to 6 nm in length. In some embodiments, the antigen binding protein may be 1, 2, 3, 4, 5, 6, 7, 8, 9, or 10 nm in length.

In some embodiments, the antigen binding protein may be about 9, about 11, about 13, about 15, about 17, about 19, about 21, about 25, or about 30 kDa in molecular weight. In some embodiments, the antigen binding protein may be at most 9, at most 11, at most 13, at most 15, at most 17, at most 19, at most 21, at most 25, or at most 30 kDa in molecular weight. In some embodiments, the antigen binding protein may be at least 9, at least 11, at least 13, at least 15, at least 17, at least 19, at least 21, at least 25, or at least 30 kDa in molecular weight. In some embodiments, the antigen binding protein may be between two values described above, for example between about 9 to about 30, about 11 to about 25, about 13 to about 21, about 15 to about 19, or about 17 to about 30 kDa in molecular weight. In some embodiments, the antigen binding protein may be between two values described above, for example between 9 to 30, 11 to 25, 13 to 21, 15 to 19, or 17 to 30 kDa in molecular weight. In some embodiments, the antigen binding protein may be 9, 11, 13, 15, 17, 19, 21, 25, or 30 kDa in molecular weight.

In some embodiments, the antigen binding protein comprises about 5, about 10, about 20, about 30, about 40, about 50, about 60, about 70, about 80, about 90, about 100, about 110, about 120, about 130, about 140, about 150, about 200, or about 250 amino acids. In some embodiments, the antigen binding protein comprises at most 5, at most 10, at most 20, at most 30, at most 40, at most 50, at most 60, at most 70, at most 80, at most 90, at most 100, at most 110, at most 120, at most 130, at most 140, at most 150, at most 200, or at most 250 amino acids. In some embodiments, the antigen binding protein comprises at least 5, at least 10, at least 20, at least 30, at least 40, at least 50, at least 60, at least 70, at least 80, at least 90, at least 100, at least 110, at least 120, at least 130, at least 140, at least 150, at least 200, or at least 250 amino acids. In some embodiments, the antigen binding protein comprises a number of amino acids between two values described above, for example between about 5 to about 250, about 10 to about 200, about 20 to about 150, about 30 to about 140, about 50 to about 130, about 60 to about 120, about 70 to about 110, about 80 to about 100, or about 90 to about 250 amino acids. In some embodiments, the antigen binding protein comprises a number of amino acids between two values described above, for example between 5 to 250, 10 to 200, 20 to 150, 30 to 140, 50 to 130, 60 to 120, 70 to 110, 80 to 100, or 90 to 250 amino acids. In some embodiments, the antigen binding protein comprises 5, 10, 20, 30, 40, 50, 60, 70, 80, 90, 100, 110, 120, 130, 140, 150, 200, or 250 amino acids.

In some embodiments, the nanobody may be about 1, about 2, about 3, about 4, about 5, about 6, about 7, about 8, about 9, or about 10 nm in length. In some embodiments, the nanobody may be at most 1, at most 2, at most 3, at most 4, at most 5, at most 6, at most 7, at most 8, at most 9, or at most 10 nm in length. In some embodiments, the nanobody may be at least 1, at least 2, at least 3, at least 4, at least 5, at least 6, at least 7, at least 8, at least 9, or at least 10 nm in length. In some embodiments, the nanobody may be between two values described above, for example between about 1 to about 10, about 2 to about 9, about 3 to about 8, about 4 to about 7, or about 5 to about 6 nm in length. In some embodiments, the nanobody may be between two values described above, for example between 1 to 10, 2 to 9, 3 to 8, 4 to 7, or 5 to 6 nm in length. In some embodiments, the nanobody may be 1, 2, 3, 4, 5, 6, 7, 8, 9, or 10 nm in length.

In some embodiments, the nanobody may be about 9, about 11, about 13, about 15, about 17, about 19, or about 21 kDa in molecular weight. In some embodiments, the nanobody may be at most 9, at most 11, at most 13, at most 15, at most 17, at most 19, or at most 21 kDa in molecular weight. In some embodiments, the nanobody may be at least 9, at least 11, at least 13, at least 15, at least 17, at least 19, or at least 21 kDa in molecular weight. In some embodiments, the nanobody may be between two values described above, for example between about 9 to about 21, about 11 to about 19, about 13 to about 17, about 15 to about 21, or about 12 to about 15 kDa in molecular weight. In some embodiments, the nanobody may be between two values described above, for example between 9 to 21, 11 to 19, 13 to 17, 15 to 21, or 12 to 15 kDa in molecular weight. In some embodiments, the nanobody may be 9, 11, 13, 15, 17, 19, or 21 kDa in molecular weight.

In some embodiments, the nanobody comprises about 70, about 80, about 90, about 100, about 110, about 120, about 130, about 140, or about 150 amino acids. In some embodiments, the nanobody comprises at most 70, at most 80, at most 90, at most 100, at most 110, at most 120, at most 130, at most 140, or at most 150 amino acids. In some embodiments, the nanobody comprises at least 70, at least 80, at least 90, at least 100, at least 110, at least 120, at least 130, at least 140, or at least 150 amino acids. In some embodiments, the nanobody comprises a number of amino acids between two values described above, for example between about 70 to about 150, about 80 to about 140, about 90 to about 130, about 100 to about 120, or about 110 to about 150 amino acids. In some embodiments, the nanobody comprises a number of amino acids between two values described above, for example between 70 to 150, 80 to 140, 90 to 130, 100 to 120, or 110 to 150 amino acids. In some embodiments, the nanobody comprises 70, 80, 90, 100, 110, 120, 130, 140, or 150 amino acids.

In some embodiments, the antigen binding protein comprises a multi-specific protein. In some embodiments, the antigen binding protein may be conjugated with a protein or peptide.

In some embodiments, the small peptide or protein binder comprises a modified amino acid or non-natural amino acid, or a modified non-natural amino acid, or a combination thereof. In some embodiments, the modified amino acid or a modified non-natural amino acid comprises a post-translational modification. In some embodiments, the small peptide comprises a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to the small peptide including the peptide backbone, the amino acid side chains, and the terminus.

In some embodiments, the allergen binding protein comprises at least one modified amino acid. In some embodiments, the allergen binding protein comprises about 1, about 2, about 3, about 4, about 5, about 6, about 7, about 8, about 9, about 10, about 11, about 12, about 13, about 14, about 15, about 20, about 25, about 30, about 35, about 40, about 45, about 50, about 60, about 65, or about 70 modified amino acids. In some embodiments, the allergen binding protein comprises at most 1, at most 2, at most 3, at most 4, at most 5, at most 6, at most 7, at most 8, at most 9, at most 10, at most 11, at most 12, at most 13, at most 14, at most 15, at most 20, at most 25, at most 30, at most 35, at most 40, at most 45, at most 50, at most 60, at most 65, or at most 70 modified amino acids. In some embodiments, the allergen binding protein comprises at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 60, at least 65, or at least 70 modified amino acids. In some embodiments, the allergen binding protein comprises about 1 to about 70, about 2 to about 65, about 3 to about 60, about 4 to about 50, about 5 to about 45, about 6 to about 40, about 7 to about 35, about 8 to about 30, about 9 to about 25, about 10 to about 20, or about 11 to about 15 modified amino acids. In some embodiments, the allergen binding protein comprises 1 to 70, 2 to 65, 3 to 60, 4 to 50, 5 to 45, 6 to 40, 7 to 35, 8 to 30, 9 to 25, 10 to 20, or 11 to 15 modified amino acids. In some embodiments, the allergen binding protein comprises 1, 2, 3, 4, 5, 6, 7, 8, 9, 10, 11, 12, 13, 14, 15, 20, 25, 30, 35, 40, 45, 50, 60, 65, or 70 modified amino acids.

In some embodiments, the modified amino acid comprises a non-canonical amino acid. In some embodiments, the non-canonical amino acid is selected from the group consisting of para-benzoylphenylalanine, 3,4-dyhydroxyphenylalanine, a tetrazine, a clooctene, homopropargylglycine, para-proparglyoxyphenylalanine, para-azidophenylalanine, para-isothiocynate phenylalanine, para-benzoylphenylalanine, para-cyanophenylalanine, para-nitrophenylalanine, a m-halogenated tyrosine analog, a halogenated proline analog, a halogenated tryptophan analog, and a halogenated leucine analog. In some embodiments, the non-canonical amino acid is para-benzoylphenylalanine. In some embodiments, the non-canonical amino acid is 3,4-dyhydroxyphenylalanine. In some embodiments, the nan-canonical amino acid is a tetrazine. In some embodiments, the non-canonical amino acid is a clooctene. In some embodiments, the non-canonical amino acid is homopropargylglycine. In some embodiments, the non-canonical amino acid is para-proparglyoxyphenylalanine. In some embodiments, the non-canonical amino acid is para-azidophenylalanine. In some embodiments, the non-canonical amino acid is para-isothiocynate phenylalanine. In some embodiments, the non-canonical amino acid is para-benzoylphenylalanine. In some embodiments, the non-canonical amino acid is para-cyanophenylalanine. In some embodiments, the non-canonical amino acid is para-nitrophenylalanine. In some embodiments, the non-canonical amino acid is a m-halogenated tyrosine analog. In some embodiments, the non-canonical amino acid is a halogenated proline analog. In some embodiments, the non-canonical amino acid is a halogenated tryptophan analog. In some embodiments, the non-canonical amino acid is a halogenated leucine analog.

In some embodiments, the solubility of the allergen binding protein is increased by about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 150%, about 200%, about 250%, about 300%, about 350%, about 400%, about 450%, about 500%, about 600%, about 700%, about 800%, about 900%, about 1000% or more compared to an unmodified allergen binding protein. In some embodiments, the solubility of the allergen binding protein is increased by at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 90%, at most 100%, at most 150%, at most 200%, at most 250%, at most 300%, at most 350%, at most 400%, at most 450%, at most 500%, at most 600%, at most 700%, at most 800%, at most 900%, at most 1000% or more compared to an unmodified allergen binding protein. In some embodiments, the solubility of the allergen binding protein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 100%, at least 150%, at least 200%, at least 250%, at least 300%, at least 350%, at least 400%, at least 450%, at least 500%, at least 600%, at least 700%, at least 800%, at least 900%, at least 1000% or more compared to an unmodified allergen binding protein. In some embodiments, the solubility of the allergen binding protein is increased by about 5% to about 1000%, about 10% to about 900%, about 15% to about 800%, about 20% to about 700%, about 25% to about 600%, about 30% to about 500%, about 35% to about 400%, about 40% to about 300%, about 45% to about 200%, about 50% to about 100%, about 55% to about 90%, or about 60% to about 80% compared to an unmodified allergen binding protein. In some embodiments, the solubility of the allergen binding protein is increased by 5% to1000%, 10% to 900%, 15% to 800%, 20% to 700%, 25% to 600%, 30% to 500%, 35% to 400%, 40% to 300%, 45% to 200%, 50% to 100%, 55% to 90%, or 60% to 80% compared to an unmodified allergen binding protein. In some embodiments, the solubility of the allergen binding protein is increased by 5%, 10%, 15%, 20%, 25%, 30%, 35%, 40%, 45% 50%, 55%, 60%, 65%, 70%, 75%, 80%, 90%, 100%, 150%, 200%, 250%, 300%, 350%, 400%, 450%, 500%, 600%, 700%, 800%, 900%, 1000% or more compared to an unmodified allergen binding protein.

In some embodiments, the binding affinity of the allergen binding protein is increased by about 10%, about 15%, about 20%, about 25%, about 50%, about 75%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, about 1000%, about 1500%, about 2000%, about 3000% or more compared to an unmodified allergen binding protein. In some embodiments, at most the binding affinity of the allergen binding protein is increased by at most 10%, at most 15%, at most 20% 25%, at most 50%, at most 75%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, at most 1000%, at most 1500%, at most 2000%, at most 3000% or more compared to an unmodified allergen binding protein. In some embodiments, at least the binding affinity of the allergen binding protein is increased by at least 10%, at least 15%, at least 20% 25%, at least 50%, at least 75%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 1500%, at least 2000%, at least 3000% or more compared to an unmodified allergen binding protein. In some embodiments, the binding affinity of the allergen binding protein is increased by about 10% to about 3000%, about 15% to about 2000%, about 20% to about 1500%, about 25% to about 1000%, about 50% to about 950%, about 75% to about 900%, about 100% to about 850%, about 150% to about 800%, about 200% to about 750%, about 250% to about 700%, about 300% to about 650%, about 350% to about 600%, about 400% to about 550%, or about 450% to about 500% compared to an unmodified allergen binding protein. In some embodiments, the binding affinity of the allergen binding protein is increased by 10% to 3000%, 15% to 2000%, 20% to 1500%, 25% to 1000%, 50% to 950%, 75% to 900%, 100% to 850%, 150% to 800%, 200% to 750%, 250% to 700%, 300% to 650%, 350% to 600%, 400% to 550%, or 450% to 500% compared to an unmodified allergen binding protein. In some embodiments, the binding affinity of the allergen binding protein is increased by 10%, 15%, 20% 25%, 50%, 75%, 100%, 150%, 200%, 250%, 300%, 350%, 400%, 450%, 500%, 550%, 600%, 650%, 700%, 750%, 800%, 850%, 900%, 950%, 1000%, 1500%, 2000%, 3000% or more compared to an unmodified allergen binding protein.

In some embodiments, the thermal stability of the allergen binding protein is increased by about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 150%, about 200%, about 250%, about 300%, about 350%, about 400%, about 450%, about 500%, about 600%, about 700%, about 800%, about 900%, about 1000% or more compared to an unmodified allergen binding protein. In some embodiments, the thermal stability of the allergen binding protein is increased by at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 90%, at most 100%, at most 150%, at most 200%, at most 250%, at most 300%, at most 350%, at most 400%, at most 450%, at most 500%, at most 600%, at most 700%, at most 800%, at most 900%, at most 1000% or more compared to an unmodified allergen binding protein. In some embodiments, the thermal stability of the allergen binding protein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 100%, at least 150%, at least 200%, at least 250%, at least 300%, at least 350%, at least 400%, at least 450%, at least 500%, at least 600%, at least 700%, at least 800%, at least 900%, at least 1000% or more compared to an unmodified allergen binding protein. In some embodiments, the thermal stability of the allergen binding protein is increased by about 5% to about 1000%, about 10% to about 900%, about 15% to about 800%, about 20% to about 700%, about 25% to about 600%, about 30% to about 500%, about 35% to about 400%, about 40% to about 300%, about 45% to about 200%, about 50% to about 100%, about 55% to about 90%, or about 60% to about 80% compared to an unmodified allergen binding protein. In some embodiments, the thermal stability of the allergen binding protein is increased by 5% to 1000%, 10% to 900%, 15% to 800%, 20% to 700%, 25% to 600%, 30% to 500%, 35% to 400%, 40% to 300%, 45% to 200%, 50% to 100%, 55% to 90%, or 60% to 80% compared to an unmodified allergen binding protein. In some embodiments, the thermal stability of the allergen binding protein is increased by 5%, 10%, 15%, 20%, 25%, 30%, 35%, 40%, 45% 50%, 55%, 60%, 65%, 70%, 75%, 80%, 90%, 100%, 150%, 200%, 250%, 300%, 350%, 400%, 450%, 500%, 600%, 700%, 800%, 900%, 1000% or more compared to an unmodified allergen binding protein.

In some embodiments, the allergen binding protein is multivalent. In some embodiments, the allergen binding protein is bivalent. In some embodiments, the allergen binding protein is trivalent. In some embodiments, the allergen binding protein is quadrivalent. In some embodiments, the solubility of the allergen binding protein is increased by about 5%, about 10%, about 15%, about 20%, about 25%, about 30%, about 35%, about 40%, about 45%, about 50%, about 55%, about 60%, about 65%, about 70%, about 75%, about 80%, about 90%, about 100%, about 150%, about 200%, about 250%, about 300%, about 350%, about 400%, about 450%, about 500%, about 600%, about 700%, about 800%, about 900%, about 1000% or more compared to a monovalent allergen binding protein. In some embodiments, the solubility of the allergen binding protein is increased by at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 90%, at most 100%, at most 150%, at most 200%, at most 250%, at most 300%, at most 350%, at most 400%, at most 450%, at most 500%, at most 600%, at most 700%, at most 800%, at most 900%, at most 1000% or more compared to a monovalent allergen binding protein. In some embodiments, the solubility of the allergen binding protein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 100%, at least 150%, at least 200%, at least 250%, at least 300%, at least 350%, at least 400%, at least 450%, at least 500%, at least 600%, at least 700%, at least 800%, at least 900%, at least 1000% or more compared to a monovalent allergen binding protein. In some embodiments, the solubility of the allergen binding protein is increased by about 5% to about 1000%, about 10% to about 900%, about 15% to about 800%, about 20% to about 700%, about 25% to about 600%, about 30% to about 500%, about 35% to about 400%, about 40% to about 300%, about 45% to about 200%, about 50% to about 100%, about 55% to about 90%, or about 60% to about 80% compared to a monovalent allergen binding protein. In some embodiments, the solubility of the allergen binding protein is increased by 5% to1000%, 10% to 900%, 15% to 800%, 20% to 700%, 25% to 600%, 30% to 500%, 35% to 400%, 40% to 300%, 45% to 200%, 50% to 100%, 55% to 90%, or 60% to 80% compared to a monovalent allergen binding protein. In some embodiments, the solubility of the allergen binding protein is increased by 5%, 10%, 15%, 20%, 25%, 30%, 35%, 40%, 45% 50%, 55%, 60%, 65%, 70%, 75%, 80%, 90%, 100%, 150%, 200%, 250%, 300%, 350%, 400%, 450%, 500%, 600%, 700%, 800%, 900%, 1000% or more compared to a monovalent allergen binding protein.

In some embodiments, the small peptide may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 28, about 30, about 32, or about 35 kDa in molecular weight. In some embodiments, the small peptide may be at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 28, at most 30, at most 32, or at most 35 kDa in molecular weight. In some embodiments, the small peptide may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 28, at least 30, at least 32, or at least 35 kDa in molecular weight. In some embodiments, the small peptide may be between two values described above, for example between about 1 to about 35, about 2 to about 34, about 3 to about 33, about 4 to about 32, about 5 to about 31, about 6 to about 30, about 7 to about 29, about 8 to about 28, about 9 to about 27, about 10 to about 26, about 11 to about 25, about 12 to about 24, about 13 to about 23, about 14 to about 21, about 15 to about 20, about 16 to about 19, about 17 to about 18 kDa in molecular weight. In some embodiments, the small peptide may be 1, 2, 3, 4, 5, 6, 7, 8, 9, 10, 11, 12, 13, 14, 15, 16, 17, 18, 19, 20, 21, 22, 23, 24, 25, 26, 28, 30, 32, or 35 kDa in molecular weight.

In some embodiments, the small peptide comprises about 5, about 10, about 20, about 60, about 120, about 140, about 160, about 180, about 200, about 220, about 240, about 260, about 280, about 300, about 320, about 340, about 360, about 380, or 400 amino acids in length. In some embodiments, the small peptide comprises at most 5, at most 10, at most 20, at most 60, at most 120, at most 140, at most 160, at most 180, at most 200, at most 220, at most 240, at most 260, at most 280, at most 300, at most 320, at most 340, at most 360, at most 380, or at most 400 amino acids in length. In some embodiments, the small peptide comprises at least 5, at least 10, at least 20, at least 60, at least 120, at least 140, at least 160, at least 180, at least 200, at least 220, at least 240, at least 260, at least 280, at least 300, at least 320, at least 340, at least 360, at least 380, or at least 400 amino acids in length. In some embodiments, the small peptide comprises a number of amino acids between two values described above, for example between about 5 to about 400, about 10 to about 380, about 20 to about 360, about 60 to about 340, about 120 to about 320, about 140 to about 300, about 160 to about 280, about 180 to about 260, or about 200 to about 240 amino acids in length. In some embodiments, the small peptide comprises a number of amino acids between 5 to 400, 10 to 80, 20 to 360, 60 to 340, 120 to 320, 140 to 300, 160 to 280, 180 to 260, or 200 to 240 amino acids in length. In some embodiments, the small peptide comprises about 5, 10, 20, 60, 120, 140, 160, 180, 200, 220, 240, 260, 280, 300, 320, 340, 360, 380, or 400 amino acids in length.

The allergen binding protein disclosed herein may be formulated as any suitable physical form. Suitable forms may include, but are not limited to an aerosol, a liquid, a gel, a semisolid, a solid, or a powder. In some embodiments, the allergen binding protein may be formulated as a toner, a cream, an emulsion, a lotion, an ointment, a paste, a gel, a suspension, a serum, an oil, a spray, a shampoo, a foam, a cleanser, a mousse, an aerosol, or a powder to be resuspended in a solvent such as water.

In some embodiments, the allergen binding protein may be formulated for coating a food. In some embodiments, the allergen binding protein may be mixed with any type of food for an animal. In some embodiments, the allergen binding protein may be provided as or added to the normal intake of food. In some embodiments, the allergen binding protein may be provided as an addition to the animal's liquid intake, including its drinking water. In some embodiments, the allergen binding protein may be formulated as a food product.

In some embodiments, the allergen binding protein may be formulated for spraying, misting, or brushing an animal or a household surface. In some embodiments, the allergen binding protein may be formulated for a humidifier and an air filter. In some embodiments, the allergen binding protein may be applied to a surface in the environment, for example by spraying, misting, depositing, wiping, or other suitable method of application to a surface. In some embodiments, the surface may be the surface of an animal that is a source of the allergen.

In some embodiments, the allergen binding protein disclosed herein may reduce, minimize, or prevent at least one symptom of an allergic response to an environmental or animal allergen. In some embodiments, the allergen binding protein disclosed herein may reduce at least one symptom of an allergic response to an environmental or animal allergen. In some embodiments, the allergen binding protein disclosed herein may minimize at least one symptom of an allergic response to an environmental or animal allergen. In some embodiments, the allergen binding protein disclosed herein may prevent at least one symptom of an allergic response to an environmental or animal allergen. In some embodiments, the allergen binding protein may contact with an environmental allergen, bind to the allergen, and prevent the allergen from inducing an allergic reaction in the subject susceptible to or suffering from allergies caused by the allergen. In some embodiments, the allergen binding protein may contact with an environmental allergen. In some embodiments, the allergen binding protein may bind to the allergen.

In some embodiments, the allergen binding protein may be resuspended or in solution at a concentration of about 0.01 milli gram per milli liter (mg/ml) to about 500 mg/ml. In some embodiments, the allergen binding protein may be at a concentration of about 0.001, about 0.005, about 0.01, about 0.05, about 0.1, about 0.5, about 1, about 5, about 10, about 20, about 30, about 40, about 50, about 60, about 70, about 80, about 90, about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, or about 600 mg/ml. In some embodiments, the allergen binding protein may be at a concentration of at most 0.001, at most 0.005, at most 0.01, at most 0.05, at most 0.1, at most 0.5, at most 1, at most 5, at most 10, at most 20, at most 30, at most 40, at most 50, at most 60, at most 70, at most 80, at most 90, at most 100, at most 120, at most 140, at most 160, at most 180, at most 200, at most 220, at most 240, at most 260, at most 280, at most 300, at most 320, at most 340, at most 360, at most 380, at most 400, at most 420, at most 440, at most 460, at most 480, at most 500, at most 520, at most 540, at most 560, at most 580, or at most 600 mg/ml. In some embodiments, the allergen binding protein may be at a concentration of at least 0.001, at least 0.005, at least 0.01, at least 0.05, at least 0.1, at least 0.5, at least 1, at least 5, at least 10, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 320, at least 340, at least 360, at least 380, at least 400, at least 420, at least 440, at least 460, at least 480, at least 500, at least 520, at least 540, at least 560, at least 580, or at least 600 mg/ml. In some embodiments, the allergen binding protein may be at a concentration of about 0.001 to about 600, about 0.005 to about 580, about 0.01 to about 560, about 0.05 to about 540, about 0.1 to about 520, about 0.5 to about 500, about 1 to about 480, about 5 to about 460, about 10 to about 440, about 20 to about 420, about 30 to about 400, about 40 to about 380, about 50 to about 360, about 60 to about 340, about 70 to about 320, about 80 to about 300, about 90 to about 280, about 100 to about 260, about 120 to about 240, about 140 to about 220, about 160 to about 200, or about 180 to about 500 mg/ml. In some embodiments, the allergen binding protein may be at a concentration of 0.001 to 600, 0.005 to 580, 0.01 to 560, 0.05 to 540, 0.1 to 520, 0.5 to 500, 1 to 480, 5 to 460, 10 to 440, 20 to 420, 30 to 400, 40 to 380, 50 to 360, 60 to 340, 70 to 320, 80 to 300, 90 to 280, 100 to 260, 120 to 240, 140 to 220, 160 to 200, or 180 to 500 mg/ml. In some embodiments, the allergen binding protein may be at a concentration of 0.001, 0.005, 0.01, 0.05, 0.1, 0.5, 1, 5, 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, or 600 mg/ml.

In some embodiments, the allergen may induce an allergic reaction in a human. In some embodiments, the allergen may comprise an environmental allergen. In some embodiments, the allergen may comprise an animal allergen. In some embodiments, the allergen may comprise a pet allergen. In some embodiments, the allergen may comprise a cat, a dog, a rabbit, a mouse, or a cockroach allergen. In some embodiments, the allergen may be a cat allergen. In some embodiments, the allergen may be a dog allergen. In some embodiments, the allergen may be a rabbit allergen. In some embodiments, the allergen may be a mouse allergen. In some embodiments, the allergen may be a cockroach allergen.

In some embodiments, the allergen may comprise a dust allergen.

In some embodiments, the allergen may comprise a plant or plant pollen allergen. In some embodiments, the plant may comprise trees, grasses, or weeds. In some embodiments, the plant or pollen allergen may be selected from the group consisting of Bet v1, Phl p 5, Phl p 1, Poa p 1, Cyn d 1, Bet v 2, Ole e 1, Amb a 1, Amb a 11, and Art v 1. In some embodiments, the plant or pollen allergen may be Bet v1. In some embodiments, the plant or pollen allergen may be Phl p 5. In some embodiments, the plant or pollen allergen may be Phl p 1. In some embodiments, the plant or pollen allergen may be Poa p 1. In some embodiments, the plant or pollen allergen may be Cyn d 1. In some embodiments, the plant or pollen allergen may be Bet v 2. In some embodiments, the plant or pollen allergen may be Ole e 1. In some embodiments, the plant or pollen allergen may be Amb a 1. In some embodiments, the plant or pollen allergen may be Amb a 11. In some embodiments, the plant or pollen allergen may be Art v 1.

In some embodiments, the allergen comprises a mold allergen. In some embodiments, the mold allergen may be selected from the group consisting of Alt a 1, Asp f 1, Asp f 2, Cla h 8, Pen ch 13, and Pen ch 18. In some embodiments, the mold allergen may be Alt a 1. In some embodiments, the mold allergen may be Asp f 1. In some embodiments, the mold allergen may be Asp f 2. In some embodiments, the mold allergen may be Cla h 8. In some embodiments, the mold allergen may be Pen ch 13. In some embodiments, the mold allergen may be Pen ch 18.

In some embodiments, the allergen comprises a food allergen. In some embodiments, the food allergen may be selected from the group consisting of Pen a 1, Ara h 1, Ara h 3. In some embodiments, the food allergen may be Pen a 1. In some embodiments, the food allergen may be Ara h 1. In some embodiments, the food allergen may be Ara h 1. In some embodiments, the food allergen may be Ara h 3.

In some embodiments, the allergen may comprise Fel d 1, Fel d 2, Fel d 3, Fel d 4, Can f1, Can f2, Can f4, Can f7, Der P1, Der P2, Ory C1, Mus M1, Bla G2, Bet v 1, Phl p 5, or a combination thereof. In some embodiments, the allergen may comprise Fel d 1. In some embodiments, the allergen may comprise Fel d 2. In some embodiments, the allergen may comprise Fel d 3. In some embodiments, the allergen may comprise Fel d 4. In some embodiments, the allergen may comprise Can f1. In some embodiments, the allergen may comprise Can f2. In some embodiments, the allergen may comprise Can f4. In some embodiments, the allergen may comprise Can f7. In some embodiments, the allergen may comprise Der P1. In some embodiments, the allergen may comprise Der P2. In some embodiments, the allergen may comprise Ory C1. In some embodiments, the allergen may comprise Mus M1. In some embodiments, the allergen may comprise Bla G2. In some embodiments, the allergen may comprise Bet v 1. In some embodiments, the allergen may comprise Phl p 5.

### Composition

In a certain aspect, this disclosure provides a composition comprising an allergen binding protein disclosed herein. In some embodiments, the composition may be a pharmaceutical composition. In some embodiments, the composition further may comprise a carrier. In some embodiments, the carrier may be a pharmaceutically acceptable carrier. The carrier may include, but is not limited to, a solvent, a stabilizing agent, a diluent, a dispersion medium, and a coating. In some embodiments, the carrier may comprise silica. In some embodiments, the composition may be in a dry form.

In some embodiments, the carrier may comprise water. In some embodiments, the composition may comprise a liquid. In some embodiments, the composition may comprise a buffer comprising a phosphate buffer or saline buffer. In some embodiments, the composition may comprise phosphate-buffered saline (PBS), citric acid buffer, carbonic acid bicarbonate buffer, sodium hydrogen phosphate-citrate buffer solution, citric acid-sodium citrate buffer, sodium hydrogen phosphate-phosphate sodium dihydrogen buffer solution, or acetic acid-sodium acetate buffer. In some embodiments, pH of the composition may be about 2, about 3, about 4, about 5, about 6, about 7, about 8, about 9, about 10, or about 11. In some embodiments, pH of the composition may be at least 2, at least 3, at least 4, at least 5, at least 6, at least 7, at least 8, at least 9, at least 10, or at least 11. In some embodiments, pH of the composition may be at most 5, at most 6, at most 7, at most 8, at most 9, at most 10, or at most 11. In some embodiments, pH of the composition may be about 2 to about 11, about 3 to about 10, or about 4 to about 9. In some embodiments, pH of the composition may be 2 to 11, 3 to 10, or 4 to 9. In some embodiments, pH of the composition may be 2, 3, 4, 5, 6, 7, 8, 9, 10, or 11.

Non-limiting examples of the carrier may comprise water, dimethyl sulfoxide (DMSO), alpha-thujene, alpha-pinene, camphene, sabinene, beta-pinene, alpha-terpinene, limonene, peltay2-carene, trans sabinene hydrate, terpinolene, 3-cyclohexen-1-ol, terpinene-4-ol, 1,2-benzenediol, linalyl acetate, borneol, bornyl acetate, alpha-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol) decanal, and a combination thereof.

In some embodiments, the composition further may comprise a preservative. In some embodiments, the preservative may comprise potassium sorbate. In some embodiments, the composition may comprise one or more preservatives. The preservatives may include, but are not limited to benzyl alcohol, methylparaben, ethyl paraben, propylparaben, butylparaben, isobutyl paraben, glycerin, ethylhexylglycerin, phenoxyethanol, sodium benzoate, ethylenediamine tetraacetic acid (EDTA), benzoic acid, phenoxyethanol, maltol, potassium sorbate, imidazolidinyl urea, diazolidinyl urea, sorbic acid, methylisothiazolinone, chlorhexidme digluconate, polyaminopropyl biguanide, sodium dehydroacetate, grapefruit seed extract, salicylic acid, DMDM hydantoin, formaldehyde, chlorphenism, triclosan, dehydroacetic acid, quaternium-15, stearalkonium chloride, zinc pyrithione, sodium metabisulfite, 2-bromo-2-nitropropane, benzalkonium chloride, sodium sulfite, sodium salicylate, citric acid, neem oil, essential oils, lactic acid, vitamin E (tocopherol), and a combination thereof.

The composition disclosed herein optionally further may comprise at least one excipient. The at least one excipient may be a pharmaceutically acceptable excipient. In some embodiments, the at least one excipient may be selected from the group consisting of: animal and vegetable fats, oils, waxes, paraffins, starch, tragacanth, cellulose derivatives, polyethylene glycols, silicones, bentonites, silicic acid, talc, zinc oxide, lactose, talc, silicic acid, aluminum hydroxide, calcium silicates, polyamide powder, and a combination thereof. In some embodiments, the at least one excipient may be naturally occurring. In another embodiments, at least one excipient may be non-naturally occurring.

The composition disclosed herein optionally further may comprise at least one additives. Non-limiting examples of the at least one additive may comprise a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, antiseptics, stabilizing agents, hydrating agents, emulsification promoters or salts and/or buffers for osmotic control, and a combination thereof. In some embodiments, the at least one additive may be naturally occurring. In another embodiments, at least one additive may be non-naturally occurring. In some embodiments, the at least one additive may comprise other useful substances. In additional embodiments, the at least one additive further may comprise absorption enhancers, permeation enhancers, thickening agents, viscosity enhancers, agents for adjusting and/or maintaining the pH, agents to adjust the osmotic pressure, preservatives, surfactants, buffers, salts, suspending agents, dispersing agents, solubilizing agents, stabilizers and/or tonicity agents.

In some embodiments, the composition further may comprise a stabilizing agent or thickener. In some embodiments, the stabilizing agent or thickener may comprise dextrin, maltodextrin, glycerol, glucose, sucrose, or trehalose. In some embodiments, the composition further may comprise an isotonic agent. In some embodiments, the stabilizing agent may be glucose, sucrose, glycerol, or trehalose.

In some embodiments, the carrier may comprise maltodextrin, dextrin, potassium sorbate, silica, water, or a combination thereof.

In another embodiments, the composition of the present invention may comprise a pharmaceutically accepted carrier. Non-limiting examples of the pharmaceutically accepted carriers may comprise a solvent, a dispersion media, a coating, an adjuvant, a stabilizing agent, a diluent, a preservative, an antibacterial and antifungal agent, an isotonic agent, or a combination thereof. In some embodiments, the diluent may comprise water, saline, dextrose, ethanol, glycerol, or a combination thereof. In some embodiments, the isotonic agent may comprise sodium chloride, dextrose, mannitol, sorbitol, lactose, or a combination thereof.

In some embodiments, the carrier may be at a concentration of at most about 80% weight per volume (wt/v, grams per milliliter of solvent) of the allergen binding protein. In some embodiments, the carrier may be at a concentration of about 50, about 55, about 60, about 65, about 70, about 75, about 80, about 85, about 90, or about 95 wt/v % of the composition. In some embodiments, the carrier may be at a concentration of at most 50, at most 55, at most 60, at most 65, at most 70, at most 75, at most 80, at most 85, at most 90, or at most 95 wt/v % of the composition. In some embodiments, the carrier may be at a concentration of at least 0.01, at least 0.05, at least 0.1, at least 0.5, at least 1, at least 2, at least 5, at least 10, or at least 20 wt/v % of the composition. In some embodiments, the carrier may be at a concentration of about 0.01 to about 95, about 0.1 to about 90, about 0.5 to about 85, about 1 to about 80, about 5 to about 75, about 10 to about 70, or about 20 to about 65 wt/v % of the composition. In some embodiments, the carrier may be at a concentration of 0.01 to 95, 0.1 to 90, 0.5 to 85, 1 to 80, 5 to 75, 10 to 70, or 20 to 65 wt/v % of the composition. In some embodiments, the carrier may be at a concentration of 50, 55, 60, 65, 70, 75, 80, 85, 90, or 95 wt/v % of the composition.

In some embodiments, the composition comprises one or more allergen binding proteins disclosed herein. In some embodiments, the composition comprises two allergen binding proteins disclosed herein. In some embodiments, the composition comprises three or more allergen binding proteins disclosed herein.

The composition may be formulated as any suitable physical form. Suitable forms may include, but are not limited to an aerosol, a liquid, a gel, a semisolid, a solid, or a powder. In some embodiments, the composition may be formulated as a toner, a cream, an emulsion, a lotion, an ointment, a paste, a gel, a suspension, a serum, an oil, a spray, a shampoo, a foam, a cleanser, a mousse, an aerosol, or a powder to be resuspended in a solvent such as water.

In some embodiments, the composition may be formulated for coating a food. In some embodiments, the composition may be edible. In some embodiments, the composition may be mixed with any type of food for an animal. In some embodiments, the composition may be provided as or added to the normal intake of food. In some embodiments, the composition may be provided as an addition to the animal's liquid intake, including its drinking water. In some embodiments, the composition may be formulated as a food product. In some embodiments, the food is a pet food. In some embodiments, the allergen is a pet allergen. In some embodiments, the allergen binding protein is formulated to be applied over the food as a topper. In some embodiments, the allergen binding protein is formulated to be mixed in with the food.

In some embodiments, the composition may be formulated for spraying, misting, or brushing an animal or a household surface. In some embodiments, the composition may be formulated for a humidifier and an air filter. In some embodiments, the composition may be applied to a surface in the environment, for example by spraying, misting, depositing, wiping, or other suitable method of application to a surface. In some embodiments, the surface may be the surface of an animal that is a source of the allergen.

In some embodiments, the composition disclosed herein may reduce, minimize, or prevent at least one symptom of an allergic response to an environmental or animal allergen. In some embodiments, the composition disclosed herein may reduce at least one symptom of an allergic response to an environmental or animal allergen. In some embodiments, the composition disclosed herein may minimize at least one symptom of an allergic response to an environmental or animal allergen. In some embodiments, the composition disclosed herein may prevent at least one symptom of an allergic response to an environmental or animal allergen. In some embodiments, the composition may contact with an environmental allergen, bind to the allergen, and prevent the allergen from inducing an allergic reaction in the subject susceptible to or suffering from allergies caused by the allergen. In some embodiments, the composition may contact with an environmental allergen. In some embodiments, the allergen binding protein may bind to the allergen.

### Methods

In certain aspects, the present disclosure provides a method of reducing, minimizing, or preventing at least one symptom of an allergic response to an environmental allergen. In some embodiments, the present disclosure provides a method of reducing at least one symptom of an allergic response to an environmental allergen. In some embodiments, the present disclosure provides a method of minimizing at least one symptom of an allergic response to an environmental allergen. In some embodiments, the present disclosure provides a method of preventing at least one symptom of an allergic response to an environmental allergen. The method may include neutralizing an allergen. Non-limiting examples of the symptom of an allergic response may include congestion, itchy nose or throat, sneezing, a runny nose and itchy, watery eyes, pain or tenderness around your cheeks, eyes or forehead, coughing, wheezing or breathlessness, itchy skin or a raised rash (hives), feeling or being sick, swollen eyes, lips, mouth or throat, a weak and rapid pulse, nausea, vomiting or diarrhea, dizziness or fainting, or a combination thereof. In some embodiments, the composition comprising the allergen binding protein may contact with an environmental allergen, bind to the allergen, and prevent the allergen from inducing an allergic reaction in the subject susceptible to or suffering from allergies caused by the allergen. In some embodiments, the method comprises administering to a subject an allergen binding protein as disclosed herein. In some embodiments, the method comprises administering to the subject a composition comprising an allergen binding protein as disclosed herein.

In some embodiments, the method may comprise spraying or misting the composition disclosed herein or contacting the surface with any one of the allergen binding proteins or compositions disclosed herein after resuspending the allergen binding protein or composition in a solvent disclosed herein. In some embodiments, contacting may comprise coating or brushing. In some embodiments, the surface may comprise the allergen. In some embodiments, the surface may comprise a surface of an air filter or a humidifier. In some embodiments, the surface may comprise food. In some embodiments, the food may be consumed by an animal that is the source of the antigen.

In some embodiments, the composition may neutralize the allergen on the surface.

In some embodiments, the surface may comprise a pet accessory (e.g., collar or brush), an air filter, or an area/product where an animal can sit or walk by (e.g., a dog bed).

In some embodiments, the misting may be performed by a humidifier or a spray bottle.

In some embodiments, about 0.01 milliliter (ml) to about 20 ml of the composition sprayed per about 1 square meter of area may neutralize the allergenicity of the antigen. In some embodiments, the neutralization of the allergenicity of the antigen may be performed by about 0.001, about 0.005, about 0.01, about 0.05, about 0.1, about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 ml of the composition sprayed per about 1 square meter of area. In some embodiments, the neutralization of the allergenicity of the antigen may be performed by at most 0.001, at most 0.005, at most 0.01, at most 0.05, at most 0.1, at most 0.5, at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, or at most 30 ml of the composition sprayed per about 1 square meter of area. In some embodiments, the neutralization of the allergenicity of the antigen may be performed by at least 0.001, at least 0.005, at least 0.01, at least 0.05, at least 0.1, at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 ml of the composition sprayed per about 1 square meter of area. In some embodiments, the neutralization of the allergenicity of the antigen may be performed by about 0.001 to about 30, about 0.005 to about 29, about 0.01 to about 28, about 0.05 to about 27, about 0.1 to about 26, about 0.5 to about 25, about 1 to about 24, about 2 to about 23, about 3 to about 22, about 4 to about 21, about 5 to about 20, about 6 to about 19, about 7 to about 18, about 8 to about 17, about 9 to about 16, about 10 to about 15, about 11 to about 14, or about 12 to about 13 ml of the composition sprayed per about 1 square meter of area. In some embodiments, the neutralization of the allergenicity of the antigen may be performed by about 0.001 to 30, 0.005 to 29, 0.01 to 28, 0.05 to 27, 0.1 to 26, 0.5 to 25, 1 to 24, 2 to 23, 3 to 22, 4 to 21, 5 to 20, 6 to 19, 7 to 18, 8 to 17, 9 to 16, 10 to 15, 11 to 14, or 12 to 13 ml of the composition sprayed per about 1 square meter of area. In some embodiments, the neutralization of the allergenicity of the antigen may be performed by 0.001, 0.005, 0.01, 0.05, 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 ml of the composition sprayed per about 1 square meter of area.

In certain aspects, the present disclosure provides a method of preparing the allergen binding protein disclosed herein. In some embodiments, the method may comprise harvesting the allergen binding protein from an engineered microbe or from a secretion by the microbe. In some embodiments, the microbe may comprise a heterologous nucleic acid encoding the allergen binding protein. In some embodiments, the microbe may comprise a yeast or bacterium. In some embodiments, the microbe may comprise a bacterium. In some embodiments, the microbe may comprise a bacterium comprising E. coli. In some embodiments, the microbe may comprise a yeast comprising Pichia pastoris. In some embodiments, the allergen binding protein may be purified or concentrated from a secretion by the microbe. In some embodiments, the allergen binding protein may be secreted by the microbe through a secretion tag and purified further. In some embodiments, the allergen binding protein may be purified through affinity purification. In some embodiments, the allergen binding protein may be purified through hyper filtration. In some embodiments, purification or concentration comprises a filtration step. In some embodiments, the filtration step comprises passing the secretion through a filter of about 0.01 nm, 0.1 nm, 1 nm, 5 nm or more in size. In some embodiments, the filter is about 0.0001 nm, about 0.0005 nm, about 0.001 nm, about 0.005 nm, about 0.01 nm, about 0.05 nm, about 0.1 nm, nm, about 0.5 nm, about 1 nm, about 2 nm, about 3 nm, about 4 nm, about 5 nm, about 6 nm, about 7 nm, about 8 nm, about 9 nm, about 10 nm, or more in size. In some embodiments, the filter is at most 0.0001 nm, at most 0.0005 nm, at most 0.001 nm, at most 0.005 nm, at most 0.001 nm, at most 0.05 nm, at most 0.1 nm, nm, at most 0.5 nm, at most 1 nm, at most 2 nm, at most 3 nm, at most 4 nm, at most 5 nm, at most 6 nm, at most 7 nm, at most 8 nm, at most 9 nm, at most 10 nm, or more in size. In some embodiments, the filter is at least 0.0001 nm, at least 0.0005 nm, at least 0.001 nm, at least 0.005 nm, at least 0.01 nm, at least 0.05 nm, at least 0.1 nm, nm, at least 0.5 nm, at least 1 nm, at least 2 nm, at least 3 nm, at least 4 nm, at least 5 nm, at least 6 nm, at least 7 nm, at least 8 nm, at least 9 nm, at least 10 nm, or more in size. In some embodiments, the size of the filter may be between two values described above, for example between about 0.0001 nm to about 10 nm, about 0.0005 nm to about 9 nm, about 0.001 nm to about 8 nm, about 0.005 nm to about 7 nm, about 0.01 nm to about 6 nm, about 0.05 nm to about 5 nm, about 0.1 nm to about 4 nm, about 0.5 nm to about 3 nm, about 1 nm to about 2 nm. In some embodiments, the size of the filter may be 0.0001 nm, 0.0005 nm, 0.001 nm, 0.005 nm, 0.01 nm, 0.05 nm, 0.1 nm, nm, 0.5 nm, 1 nm, 2 nm, 3 nm, 4 nm, 5 nm, 6 nm, 7 nm, 8 nm, 9 nm, or 10 nm. In some embodiments, the method does not comprise a centrifugation step.

In some embodiments, the method further may comprise incorporating the heterologous nucleic acid encoding the allergen binding protein into a cell-free protein expression system. In some embodiments, the cell-free protein expression system comprises ribosomes.

Also provided herein is a method of treating an allergy in a subject in need thereof. In some embodiments, the method comprises administering to the subject a therapeutically effective amount of a microbe engineered to produce any one of the allergen binding proteins disclosed herein. In some embodiments, the method comprises administering to the subject a therapeutically effective amount of a microbe engineered to produce any one of the compositions comprising an allergen binding proteins disclosed herein. In some embodiments, the microbe may be a yeast. In some embodiments, the microbe may be a bacterium. In some embodiments, the microbe may comprise a bacterium. In some embodiments, the microbe may comprise a bacterium comprising E. coli. In some embodiments, the microbe may comprise a yeast comprising Pichia pastoris. In some embodiments, the microbe may be administered to the subject orally. In some embodiments, the microbe may be ingested. In some embodiments, the microbe produces the allergen binding protein or the composition when ingested.

Also provided herein is a method of neutralizing an allergen. In some embodiments, the method comprises aerosolizing any one of the allergen binding proteins described herein into a mist and contacting the mist to a surface comprising an allergen. In some embodiments, the method comprises aerosolizing any one of the compositions comprising an allergen binding protein described herein into a mist and contacting mist to a surface comprising an allergen. In some embodiments, the aerosolizing may be performed by an aerosolization machine. In some embodiments, the aerosolization machine may be worn by a subject. In some embodiments, the surface comprises a pet accessory. In some embodiments, the surface comprises an area/product where an animal can sit or walk by.

Also provided herein is a method of neutralizing an allergen. In some embodiments, embodiments, the method comprises aerosolizing any one of the allergen binding proteins described herein into a mist and contacting the mist to a food. In embodiments, the method comprises aerosolizing any one of the compositions comprising an allergen binding protein described herein into a mist and contacting the mist to a food. In some embodiments, the food is a pet food. In some embodiments, the allergen is a pet allergen. In some embodiments, the mist is applied over the food as a topper. In some embodiments, the mist is mixed into the food.

Also provided herein is a method of neutralizing an allergen. In some embodiments, the method comprises contacting any one of the allergen binding proteins described herein to a food. In some embodiments, the method comprises contacting any one of the compositions comprising an allergen binding protein described herein to a food. In some embodiments, the food is a pet food. In some embodiments, the allergen is a pet allergen. In some embodiments, the allergen binding protein is applied over the food as a topper. In some embodiments, the allergen binding protein is mixed into the food. In some embodiments, the composition is applied over the food as a topper. In some embodiments, the composition is mixed into the food.

Also provided herein is a method of treating an allergy in a subject in need thereof. In some embodiments, the method comprises aerosolizing any one of the allergen binding proteins described herein into a mist. In some embodiments, the method comprises aerosolizing any one of the compositions comprising an allergen binding protein described herein into a mist. In some embodiments, the aerosolizing may be performed by an aerosolization machine. In some embodiments, the aerosolization machine may be worn by a subject. In some embodiments, the method further comprises inhalation of the mist by the subject. In some embodiments, the subject may be a human. In some embodiments, the subject may be a non-human animal.

Also provided herein is a method of identifying allergen binding proteins. In some embodiments, the method comprises transfecting target cells with human Fc epsilon receptor I (FceR1). In some embodiments the method comprises contacting the target cells with an IgE isolated from a pet-allergic subject. In some embodiments, the method comprises contacting the target cells with a candidate allergen binding protein and a pet allergen. In some embodiments, the pet allergic subject is a cat allergic subject. In some embodiments, the pet allergen is a cat allergen. In some embodiments, the cat allergen is FelD1. In some embodiments, the method comprises measuring histamine release by the target cells. In some embodiments, the method comprises measuring beta-hexosaminidase release by the target cells.

### Kits

Disclosed herein, in some embodiments, are kits for using the compositions described herein. In some embodiments, the kits disclosed herein may be used to reduce, minimize, or prevent at least one symptom of an allergic response to an environmental allergen. In some embodiments, the kits may comprise an assemblage of materials or components apart from the composition. In some embodiments, the kits may require additional of external materials comprising water.

Instructions for use may be included in the kit. In some embodiments, the kit may comprise instructions for administering the composition to a subject in need thereof. In some embodiments, the kit may comprise instructions for measure viability of the restored compositions, to ensure efficacy for its intended purpose (e.g., therapeutic efficacy if used for treating a subject).

Optionally, the kit also may comprise other useful components, such as, diluents, buffers, pharmaceutically acceptable carriers, syringes, catheters, applicators, or measuring tools, or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components may be typically contained in suitable packaging material(s).

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The expressions “at least one of A and B” and “at least one of A or B” may be construed to mean at least A, at least B, or at least A and B (i.e., a set comprising A and B, which set may include one or more additional elements). The term “A and/or B” may be construed to mean only A, only B, or both A and B.

The expressions “at least about A, B, and C” and “at least about A, B, or C” may be construed to mean at least about A, at least about B, or at least about C. The expressions “at most about A, B, and C” and “at most about A, B, or C” may be construed to mean at most about A, at most about B, or at most about C.

The expression “between about A and B, C and D, and E and F” may be construed to mean between about A and about B, between about C and about D, and between about E and about F. The expression “between about A and B, C and D, or E and F” may be construed to mean between about A and about B, between about C and about D, or between about E and about F.

The expression “about A to B and C to D” may be construed to mean between about A and about B and between about C and about D. The expression “about A to B or C to D” may be construed to mean between about A and about B or between about C and about D.

The term “exemplary” as used herein means “serving as an example, instance, or illustration.” Any embodiment described herein as “exemplary” is not to be construed as preferred or advantageous over other embodiments.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “pharmaceutically acceptable” refers to approved or approvable by a regulatory agency of the Federal or a state government or listed in the U.S. Pharmacopeia (U.S.P.) or other generally recognized pharmacopeia for use in animals, including humans. A “pharmaceutically acceptable excipient” refers to an excipient that can be administered to a subject, together with an active moiety (i.e., an allergen binding protein), and which does not destroy the pharmacological activity thereof and is nontoxic when administered in doses sufficient to deliver a therapeutic amount of the moiety. The term “pharmaceutically acceptable carrier” refers to any non-toxic substance that may be safely administered to a patient and which does not interfere with the effectiveness of the biological activity of the active ingredients. The term “pharmaceutically acceptable carrier” can refer to a “biologically compatible carrier”, which is a substance that does not interfere with the ability of an allergen binding protein to bind and is non-toxic for human or animal use. The carrier can be solid, liquid, or gas, and it helps in delivering the active ingredient in the medicine to the target area in the body. The pharmaceutically acceptable carrier may be chosen based on its compatibility with the active ingredient(s) and its suitability in the route of administration. It may include substances which serve to stabilize, solubilize, emulsify, suspend, or otherwise facilitate the functional dispersion of the active ingredient(s) in the pharmaceutical composition. Examples include, but are not limited to, binders, fillers, diluents, solvents, buffers, preservatives, surfactants, and the like, which are commonly used in the art of pharmaceutical formulation.

The term “therapeutically effective amount” refers to the quantity of a compound, a composition, or pharmaceutical agent that, when administered to a subject or patient, is sufficient to effect a beneficial therapeutic response over time. This response may include, but is not limited to, the alleviation of one or more symptoms, the modification or halt of disease progression, or the complete elimination of the disease condition. A therapeutically effective amount can vary based on various factors such as the specific condition being treated, the particular compound, composition, or pharmaceutical agent being used, the severity of the condition, the age and weight of the patient, and the route of administration. The determination of a therapeutically effective amount is within the ability of one skilled in the art, often employing a regimen of dosage administration adjusted over time.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “antibody” may include fully assembled antibodies, antibody fragments that can bind antigen, for example, Fab, F(ab′)2, Fv, single chain antibodies (scFv), diabodies, antibody chimeras, hybrid antibodies, bispecific antibodies, single VHH domains from camelids (also known as nanobodies), and the like.

The term “complementarity determining region” or “CDR” is a segment of the variable region of an antibody or allergen binding protein that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

A “nanobody” is a single domain antibody or portion of a single domain antibody that is usually derived from Alpacas, Llamas, or other camelids. The nanobody may refer to a single variable domain on a heavy chain of the antibody (VHH domain). A nanobody may be modified to increase solubility, stability, efficacy, or any other important characteristics.

A “monobody” is a synthetic binding protein derived from mutating the fibronectin type III domain (FN3). The binding interface for the FN3 domain is mutated in a monobody to develop protein binders to bind to a protein of interest.

A “DARPin” is a genetically engineered antibody mimetic protein that stands for designed ankyrin repeat proteins.

As used herein, the terms “polypeptide,” “peptide” and “protein” may be used interchangeably herein in reference to a polymer of amino acid residues. A protein may refer to a full-length polypeptide as translated from a coding open reading frame, or as processed to its mature form, while a polypeptide or peptide may refer to a degradation fragment or a processing fragment of a protein that nonetheless uniquely or identifiably maps to a particular protein. A polypeptide may be a single linear polymer chain of amino acids bonded together by peptide bonds between the carboxyl and amino groups of adjacent amino acid residues. Polypeptides may be modified, for example, by the addition of carbohydrate, phosphorylation, etc.

As used herein, the terms “fragment,” or “portion,” or equivalent terms may refer to a portion of a protein that has less than the full length of the protein and optionally maintains the function of the protein.

The term “allergen” refers to any naturally occurring protein or mixtures of proteins that have been reported to induce allergic, i.e., IgE mediated reactions upon their repeated exposure to an individual.

The section headings used herein are for organizational purposes only and are not to be construed as limiting the subject matter described.

### EXAMPLES

The following examples are included for illustrative purposes only and are not intended to limit the scope of the invention.

### Example 1: ELISA Binding Assay for Anti-Fel D1 Nanobodies

2 microgram per milliliter (μg/mL) target antigen is coated in a 96-well plate and ELISA assay is performed after incubation with candidate VHHs. The binding EC50 of each clone is calculated using Prism GraphPad. The positive control antibody is involved as an internal control.

### Example 2: Fel D1 Functional Blocking Assay

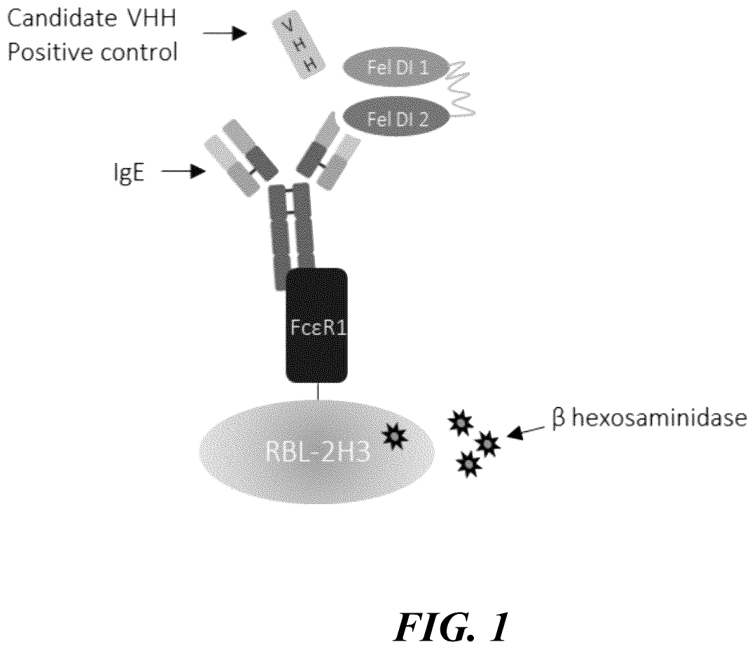

Human FcεR1 (Fc epsilon RI) is stably transfected into RBL-2H3 cells and the resulted stable cells, human IgE from cat-allergic population, cat saliva, positive control antibody and candidate VHH are used for evaluation of candidate VHH function. The EC50 of each clone is calculated using Prism GraphPad. The functional assay is schematically illustrated in FIG. 1.

### Example 3: Use of the Composition to Alleviate Allergy Symptoms

A person, who is allergic to cat or dog dander, obtains the composition disclosed herein with the nanobodies neutralizing the pet allergens. The composition is mixed with water in a bottle with an attached sprayer. The water may be tap water. The composition is well suspended in water by shaking the bottle. The composition suspended in water is sprayed in the air, around the house, and on various household surfaces comprising pet bedding, personal bedding, air filters, couch, pillows, walls, or floors. The person finds that the composition alleviates the allergy symptoms.

### Example 4: Identification of Anti-Der p2 VHH

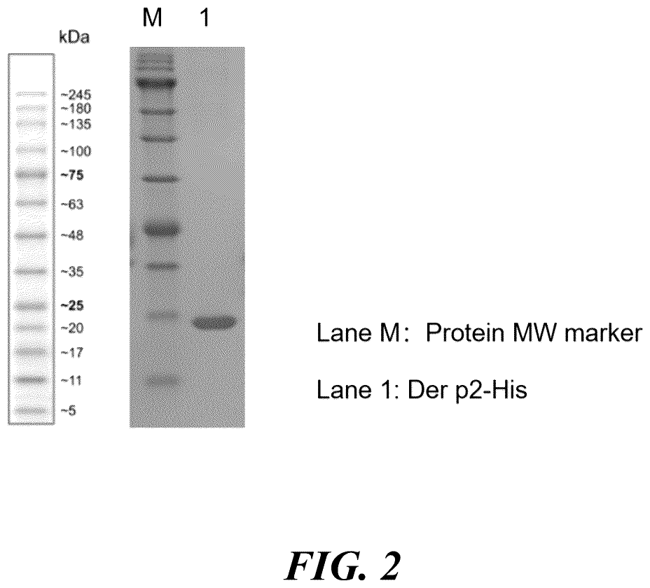

In order to obtain single variable domain on heavy chain, i.e., VHH, specific for dust mite allergen Der p2, a recombinant His-tagged Der p2 was prepared, which reached high purity, as shown via SDS-Page Western blotting in FIG. 2. To confirm this, an ELISA was performed. Briefly, the Der p2-HIS was coated onto an ELISA plate and a known Der p2 antibody (clone 6OY4) was added. A secondary detection antibody capable of binding to the Der p2 antibody was then added. The secondary detection antibody was conjugated to a horseradish peroxidase, which can be utilized to produce a colorimetric reaction. Then, optical density was measured. As summarized in Table 1, the recombinant Der p2-His was effectively bound by the antibody.

TABLE 1
Recombinant Der p2-His ELISA Binding Assay

| Der p2-His Concentration | Repeat 1 | Repeat 2 |
|---|---|---|
| (μg/mL) | (OD at 450 nm) | (OD at 450 nm) |
| 10 | 4.61 | 4.57 |
| 2 | 4.06 | 4.02 |
| 0.4 | 1.31 | 1.37 |
| 0.08 | 0.38 | 0.35 |
| 0.016 | 0.11 | 0.12 |
| 0.0032 | 0.09 | 0.10 |
| 0.00064 | 0.07 | 0.09 |
| Blank | 0.11 | 0.06 |

The recombinant Der p2 was then administered to two alpacas in four rounds. Alpacas were administered 250 ug of the recombinant Der p2 on day 0, day 14, day 28, and day 49. An ELISA using titrated serum from blood was performed detecting levels of anti-Der p2 VHH. The results of which are summarized in Table 2. Each measurement was performed in duplicate. Negative serum was taken from naïve alpacas to be used as a negative control.

TABLE 2
Der p2 Immunized Alpaca Serum Titration

| | | OD at 450 nm | OD at 450 nm |
|---|---|---|---|
| | | Day 14 (2^{nd} | Day 28 (3^{rd} |
| Titration | Negative Serum | Immunization) | Immunization) |

Alpaca 1

| | | | | | | |
|---|---|---|---|---|---|---|
| 1:1K | 0.246 | 0.255 | 3.574 | 3.468 | 3.697 | 3.739 |
| 1:2K | 0.149 | 0.154 | 3.486 | 3.365 | 3.679 | 3.623 |
| 1:4k | 0.119 | 0.116 | 3.126 | 3.020 | 3.215 | 3.231 |
| 1:8K | 0.104 | 0.099 | 2.341 | 2.308 | 2.460 | 2.383 |
| 1:16K | 0.099 | 0.093 | 1.484 | 1.521 | 1.641 | 1.601 |
| 1:32k | 0.099 | 0.081 | 0.929 | 0.887 | 1.045 | 1.063 |
| 1:64K | 0.085 | 0.081 | 0.505 | 0.491 | 0.589 | 0.648 |
| PBS | 0.124 | 0.089 | 0.095 | 0.084 | 0.091 | 0.100 |

Alpaca 2

| | | | | | | |
|---|---|---|---|---|---|---|
| 1:1K | 0.201 | 0.190 | 0.474 | 0.509 | 3.628 | 3.739 |
| 1:2K | 0.152 | 0.150 | 0.297 | 0.310 | 2.916 | 3.120 |
| 1:4k | 0.113 | 0.113 | 0.203 | 0.201 | 2.198 | 2.230 |
| 1:8K | 0.101 | 0.103 | 0.151 | 0.147 | 1.480 | 1.452 |
| 1:16K | 0.091 | 0.089 | 0.103 | 0.107 | 0.927 | 0.899 |
| 1:32k | 0.082 | 0.082 | 0.105 | 0.107 | 0.603 | 0.554 |
| 1:64K | 0.071 | 0.079 | 0.090 | 0.087 | 0.359 | 0.327 |
| PBS | 0.093 | 0.079 | 0.095 | 0.091 | 0.105 | 0.084 |

After the 3^{rd} immunization, peripheral blood mononuclear cells (PBMCs) were obtained from the immunized alpacas, RNA was extracted from the PBMCs, and RNA was reverse transcribed into cDNA. VHH sequences were then amplified from cDNA using the single domain antibody cloning primer combinations and subcloned into the yeast display vector pYDisplay, which were electro-transformed to EYB100 competent cells to construct single domain antibody yeast display libraries.

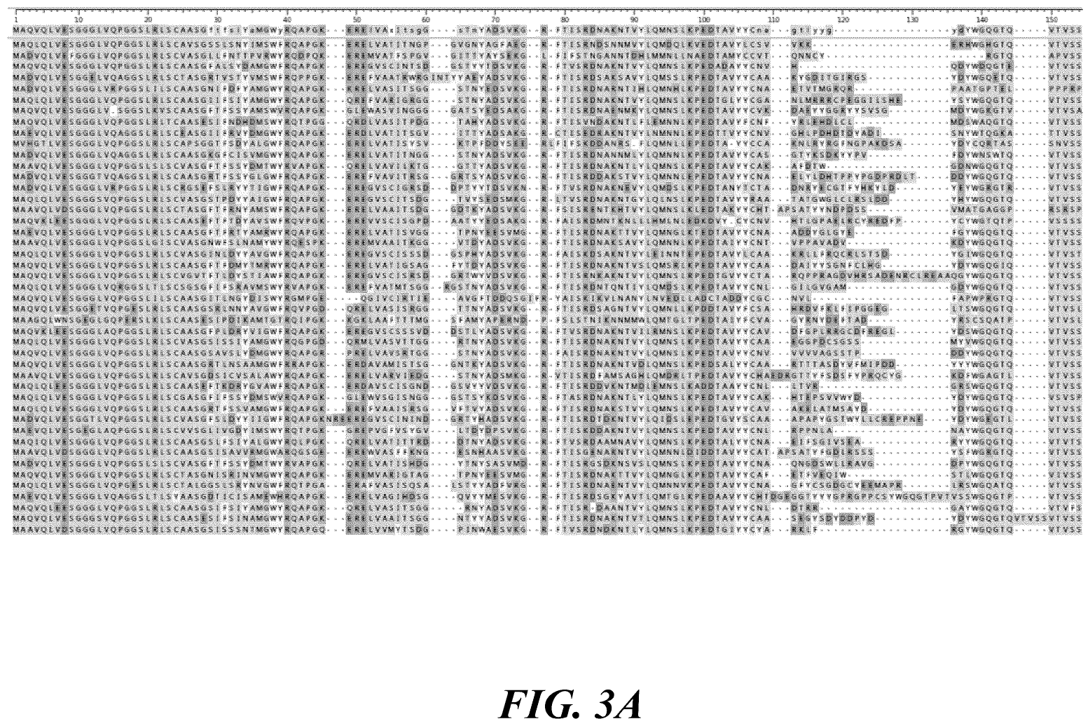

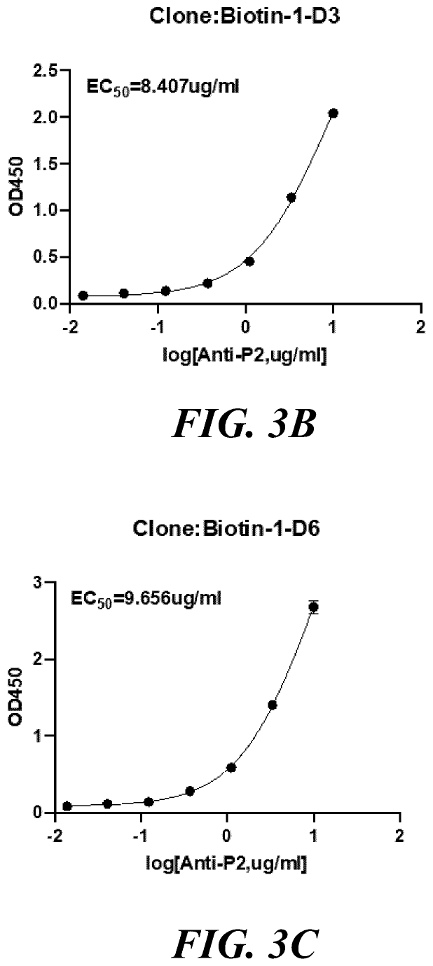

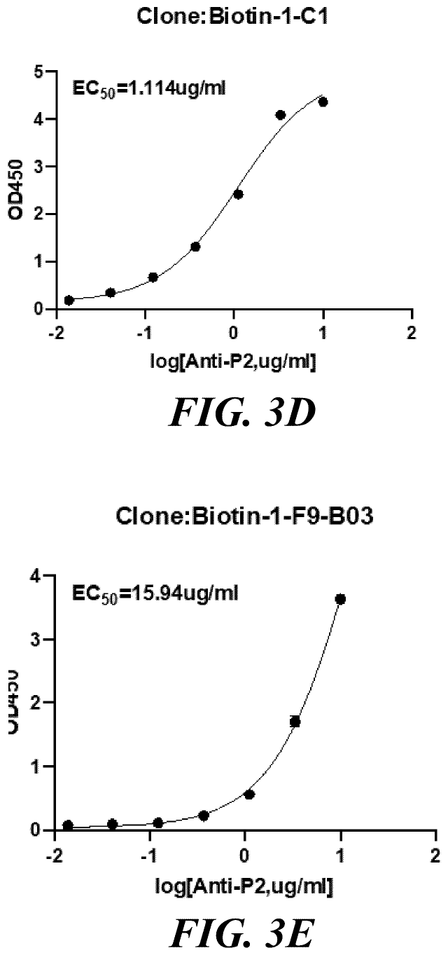

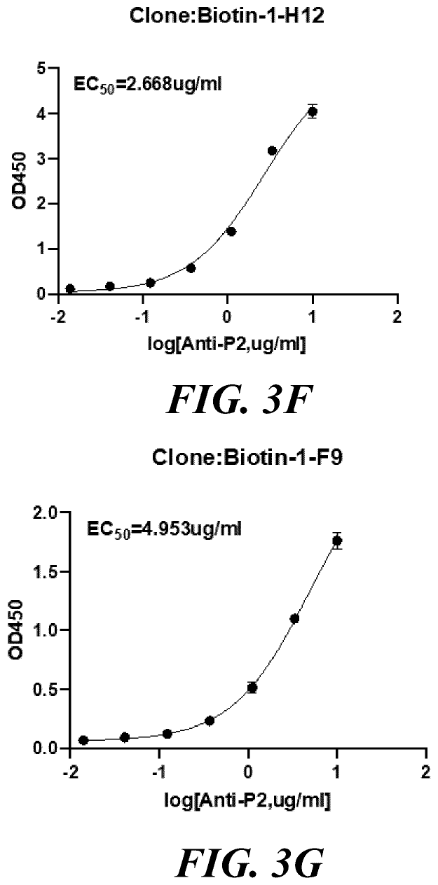

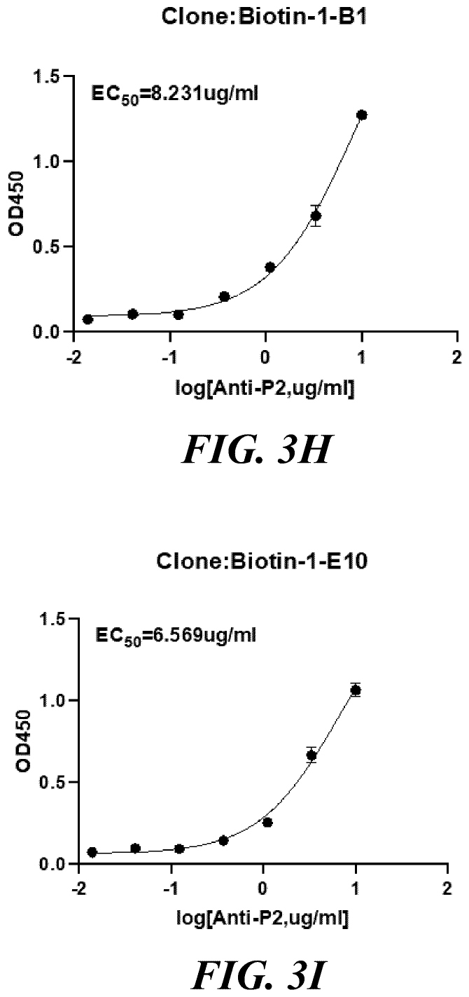

The sequences of 50 randomly selected clones are shown in FIG. 3A, which demonstrated low overlap, indicating a diverse library. Single clones were subjected to a flow cytometry-based assay in which biotinylated recombinant Der p2 was incubated with the VHH clone and following a wash step, detection of bound Der p2 identified the clone as a VHH candidate. VHH candidates were then constructed into prokaryotic expression vectors.

Isolated VHH clones were further validated by performing an ELISA assay as described in Example 1. Metrics of thermostability including melting temperature (Tₘ), onset temperature (Tₒₙₛₑₜ), and temperature of aggregation (T_{agg}), were also tested with increasing temperature in the range of 20° C.-95° C. at the speed of 1° C./min. These measurements were performed using a Nanotemper DSF. When applicable, Tₘ1 refers to the first melting point observed, Tₘ2 refers to the second melting point observed, etc., Sequence information for the VHH clones and the binding assay results are summarized in Table 3. The individual binding curves are shown for each candidate in FIGS. 3B-3I. Each clone demonstrated strong binding, indicated by low EC₅₀ values.

TABLE 3
Anti-Der p2 VHH Validation

| | | | EC₅₀ | | |
|---|---|---|---|---|---|
| | SEQ | | (μg/ | | |
| Clone | ID NO: | Sequence | mL) | T_{agg} | Tₘ |
| SDAB22 | 1 | QLQLVESGGGLVQPGGSLRLSCVVS | 8.407 | 42.45 | Tm1: |
| 111-1- | | GRTLSGHNMGWFRQAPGKEREFVA | | | 53.78 |
| D3 | | RINSNGGTTRYADAVKGRFTISRDNA | | | Tm2: |
| | | KNTAYLQMNSLKSEDTAVYYCRLV | | | 69.76 |
| | | GPAGYWGQGTQVTVSS | | | |
| SDAB22 | 2 | DVQLVESGGGLVQPGGSLRLSCVAS | 9.656 | 41.57 | Tm1: |
| 111-1- | | GLTFSYYDMKWHRQAPGKERELVA | | | 49.02 |
| D6 | | AIIEGGRTMYADSVKGRFTISRDSPK | | | Tm2: |
| | | KSVYLQMNNLKSEDTAVYYCNAVR | | | 62.55 |
| | | ARFRGLGNDDAWGQGTQVTVSS | | | |
| SDAB22 | 3 | QVQLVESGGGLVQPGGSLRLSCAAS | 1.114 | 43.53 | Tm1: |
| 111-1- | | ASFVGAYAMYWFRQATGKQRELVA | | | 54.52 |
| C1 | | FITSGNSTNYADSVKGRFTISRDYTK | | | Tm2: |
| | | NTVYLQMNKLKPEDTAVYSCNLVTS | | | 66.51 |
| | | RGDYWGQGTQVTVSS | | | |
| SDAB22 | 4 | EVQLVESGGGLVQPGGSLRLSCAAS | 15.94 | 40.04 | 71.38 |
| 111-1- | | GRTFSSYAMGWFRQAPGKEREFVAG | | | |
| F9-B03 | | ISRRGSNIYYADSVKGRFTISRDNAK | | | |
| | | NTVYLQMNSLKPEDTAVYYCNARHP | | | |
| | | RISTRLYWGQGTQVTVSS | | | |
| SDAB22 | 5 | QVQLVESGGGLVQPGGSLRLSCAAS | 2.668 | 43.37 | Tm1: |
| 111-1- | | RSIDRFPAMGWYRQAPGKKRELVAA | | | 51.93 |
| H12 | | ISSANTRTKYADSVKGRFTISRDNAE | | | Tm2: |
| | | NTMYLQMNSLKPEDTAVYYCNVFPR | | | 59.13 |
| | | PVQGYWGQGTQVTVSS | | | Tm3: |
| | | | | | 68.62 |
| SDAB22 | 6 | QVQLVESGGGLVQPGGSLRLSCAAS | 4.953 | | |
| 111-1- | | GRIFRPDGMAWYRQAPGKQREFVAG | | | |
| F9 | | ITRGGRTTYADSVKGRFTISRDNAEN | | | |
| | | TVYLQMNSLKPEDTAVYYCNAKPSA | | | |
| | | WTLPRYRYDYWGQGTQVTVSS | | | |
| SDAB22 | 7 | DVQLVESGGGLVQPGGSLRLSCAAS | 8.231 | | |
| 111-1- | | GFTFALYSMRWYRQAPGKERELVAA | | | |
| B1 | | ITSGRSTNYADSVKGRFTISRDNAKN | | | |
| | | TVYLQMNSLKPEDTAVYHCNARHPI | | | |
| | | ATLGNYWGQGTPVTVSS | | | |
| SDAB22 | 8 | QLQLVESGGGMVQPGGSLRLSCAAS | 6.569 | | |
| 111-1- | | GTLFSTALMGWYRQAPGKQRTLVAS | | | |
| E10 | | ITDGGSTNYVDSVKGRFTISRDNEKN | | | |
| | | TVYLQMNSLKFEDTAVYYCNAHWG | | | |
| | | SYDYWGQGTQVVVSS | | | |

### Example 5: Identification of Anti-can f1 VHH

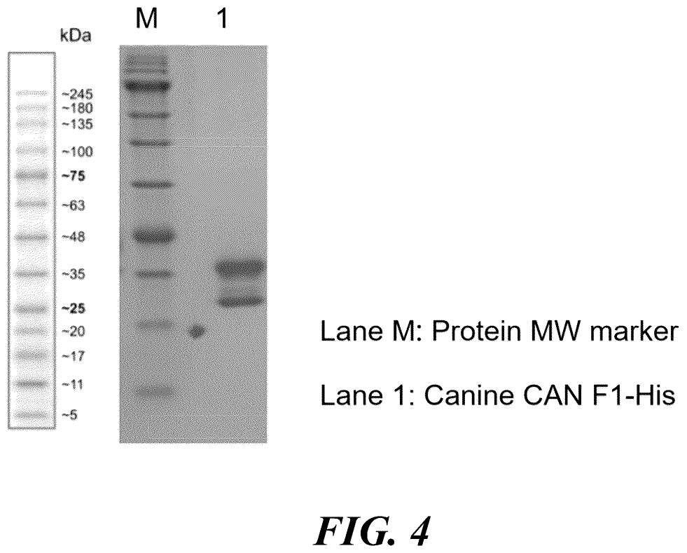

In order to obtain single variable domain on heavy chain, i.e., VHH, specific for dog allergen Can f1, a recombinant His-tagged Can f1 was prepared, which reached high purity, as shown via SDS-Page Western blotting in FIG. 4. To confirm this, an ELISA was performed measuring optical density when the recombinant Can f1-His is incubated with a biotinylated anti-His antibody. As summarized in Table 4, the recombinant Can f1-His was effectively bound by the antibody.

TABLE 4
Recombinant Can f1-His ELISA Binding Assay

| Can f1-His Concentration | Repeat 1 | Repeat 2 |
|---|---|---|
| (μg/mL) | (OD at 450 nm) | (OD at 450 nm) |
| 10 | 2.84 | 2.92 |
| 2 | 0.95 | 1.03 |
| 0.4 | 0.21 | 0.24 |
| 0.08 | 0.15 | 0.16 |
| 0.016 | 0.09 | 0.12 |
| 0.0032 | 0.09 | 0.11 |
| 0.00064 | 0.07 | 0.11 |
| Blank | 0.09 | 0.08 |

The recombinant Can f1 was then administered to two alpacas in four rounds. Alpacas were administered 250 ug of the recombinant Can f1 on day 0, day 14, day 28, and day 49. An ELISA using titrated serum from blood was performed detecting levels of anti-Can f1 VHH. The results of which are summarized in Table 5. Each measurement was performed in duplicate. Negative serum was taken from naïve alpacas to be used as a negative control.

TABLE 5
Can f1 Immunized Alpaca Serum Titration

| | | OD at 450 nm | OD at 450 nm |
|---|---|---|---|
| | | Day 14 (2^{nd} | Day 28 (3^{rd} |
| Titration | Negative Serum | Immunization) | Immunization) |

Alpaca 1

| | | | | | | |
|---|---|---|---|---|---|---|
| 1:1K | 0.294 | 0.342 | 3.972 | 4.008 | 4.040 | 4.079 |
| 1:2K | 0.182 | 0.198 | 3.917 | 3.911 | 4.055 | 4.088 |
| 1:4k | 0.137 | 0.147 | 3.505 | 3.495 | 3.962 | 3.989 |
| 1:8K | 0.116 | 0.117 | 2.724 | 2.799 | 3.873 | 3.775 |
| 1:16K | 0.101 | 0.110 | 1.925 | 2.018 | 3.329 | 3.249 |
| 1:32k | 0.102 | 0.107 | 1.243 | 1.279 | 2.684 | 2.606 |
| 1:64K | 0.103 | 0.103 | 0.765 | 0.737 | 1.881 | 1.812 |
| PBS | 0.139 | 0.111 | 0.106 | 0.108 | 0.105 | 0.106 |

Alpaca 2

| | | | | | | |
|---|---|---|---|---|---|---|
| 1:1K | 0.228 | 0.218 | 3.521 | 3.597 | 4.285 | 4.157 |
| 1:2K | 0.185 | 0.179 | 3.522 | 3.437 | 4.112 | 4.104 |
| 1:4k | 0.122 | 0.122 | 2.835 | 2.863 | 3.985 | 4.020 |
| 1:8K | 0.108 | 0.111 | 2.268 | 2.339 | 3.879 | 3.892 |
| 1:16K | 0.101 | 0.098 | 1.515 | 1.558 | 3.583 | 3.610 |
| 1:32k | 0.091 | 0.093 | 1.018 | 1.044 | 2.897 | 2.942 |
| 1:64K | 0.088 | 0.087 | 0.600 | 0.607 | 2.038 | 2.122 |
| PBS | 0.112 | 0.100 | 0.123 | 0.108 | 0.125 | 0.141 |

After the 3^{rd} immunization, peripheral blood mononuclear cells (PBMCs) were obtained from the immunized alpacas, RNA was extracted from the PBMCs, and RNA was reverse transcribed into cDNA. VHH sequences were then amplified from cDNA using the single domain antibody cloning primer combinations and subcloned into the yeast display vector pYDisplay, which were electro-transformed to EYB100 competent cells to construct single domain antibody yeast display libraries.

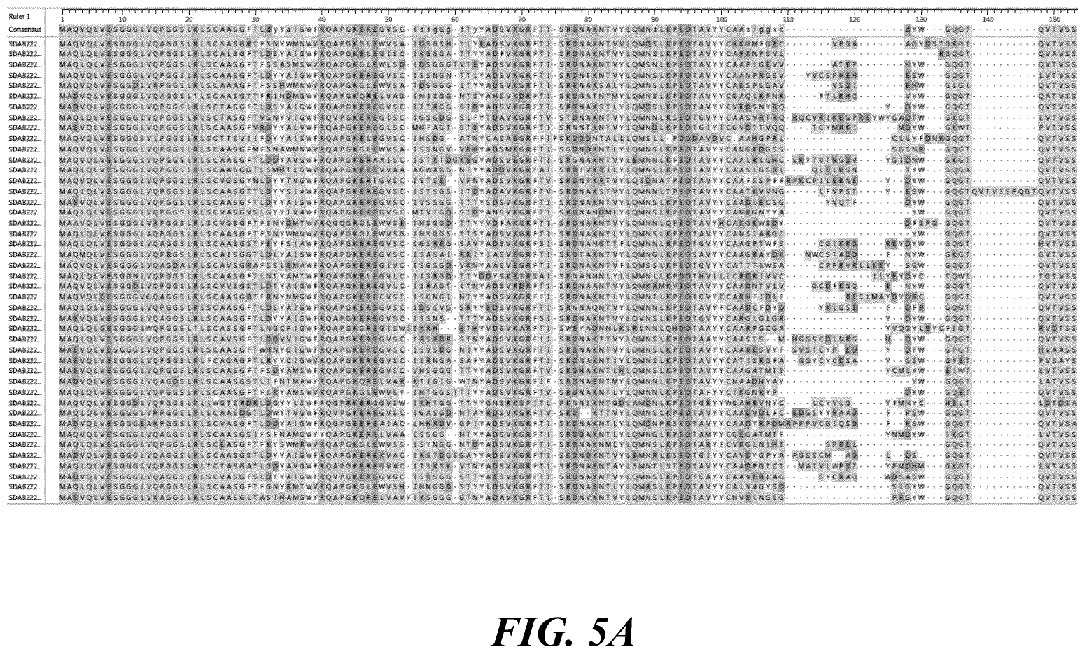

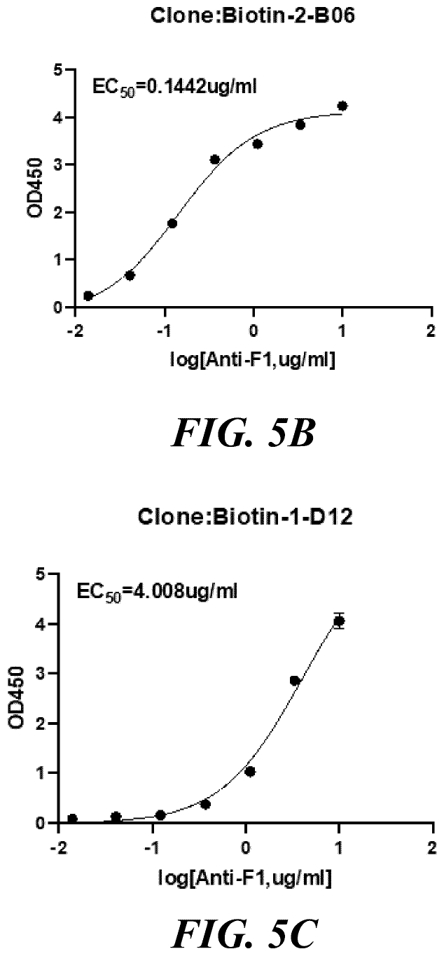

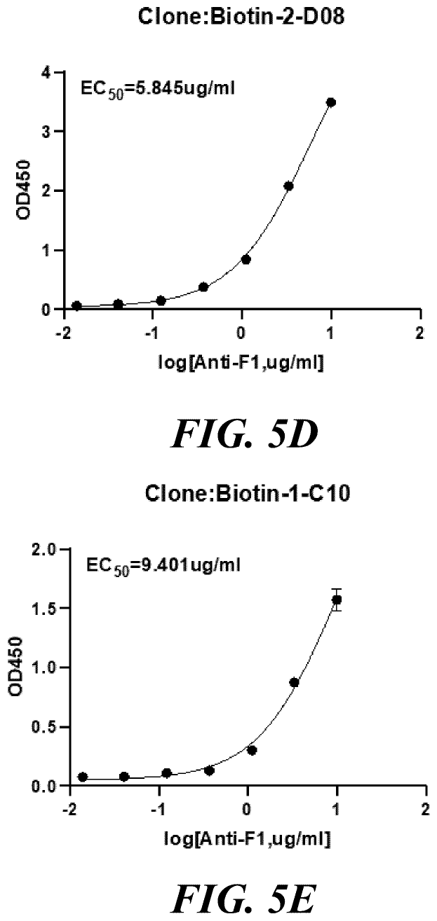

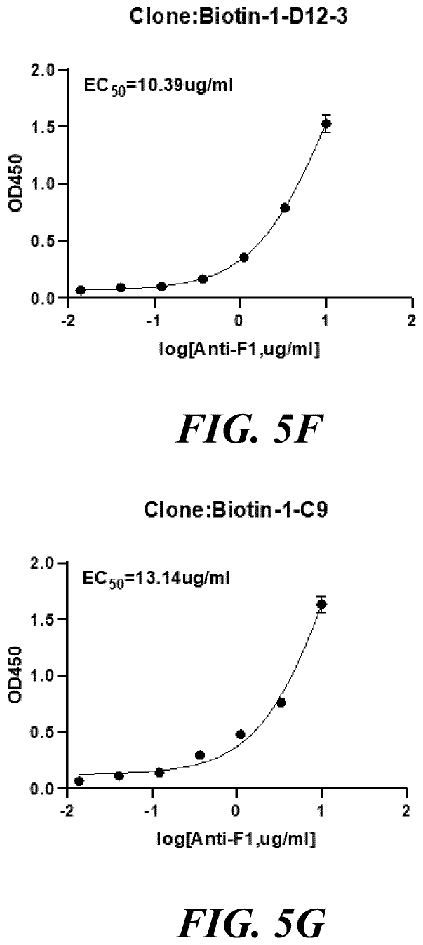

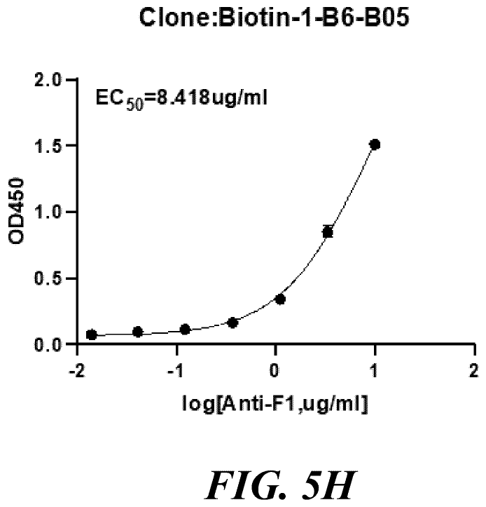

The sequences of 50 randomly selected clones are shown in FIG. 5A, which demonstrated low overlap, indicating a diverse library. Single clones were subjected to a flow cytometry-based assay in which biotinylated recombinant Can f1 was incubated with the VHH clone and following a wash step, detection of bound Can f1 identified the clone as a VHH candidate. VHH candidates were then constructed into prokaryotic expression vectors.

Isolated VHH clones were further validated by performing an ELISA assay as described in Example 1. Metrics of thermostability including melting temperature (Tₘ), onset temperature (Tₒₙₛₑₜ), and temperature of aggregation (T_{agg}), were also tested with increasing temperature in the range of 20° C.-95° C. at the speed of 1° C./min. Sequence information for the VHH clones and the binding assay results are summarized in Table 6. The individual binding curves are shown for each candidate in FIGS. 5B-5H. Each clone demonstrated strong binding, indicated by low EC₅₀ values.

TABLE 6
Anti-Can f1 VHH Validation

| | | | EC₅₀ | | |
|---|---|---|---|---|---|
| | SEQ | | (μg/ | | |
| Clone | ID NO: | Sequence | mL) | T_{agg} | Tₘ |
| SDAB22 | 67 | QVQLVESGGGLVQPGGSLRLSCAAS | 0.1442 | 48.00 | Tm1: |
| 112-2- | | GFTFRLAAMGWYRQAPEKEREWVA | | | 56.38 |
| B06 | | SITGPGTDTNYADSVKGRFTVSRDNA | | | Tm2: |
| | | KNTVYLQMNSLKPEDTAVYYCRGM | | | 63.74 |
| | | GYWGKGTLVTVSS | | | Tm3: |
| | | | | | 70.13 |
| SDAB22 | 68 | AVQLVDSGGGLVQAGGSLRLACAAS | 4.008 | 49.15 | 57.78 |
| 112-1- | | GRTFDTYAVGWFRQAPGKERDVVA | | | |
| D12 | | SITWTSGSTWYADFVKGRFTISKDNA | | | |
| | | KNTVYLQMNNLSPEDTAVYYCGAR | | | |
| | | NQIYTRWDSWGQGTQVTVSS | | | |
| SDAB22 | 69 | QVKLEESGGGLVQAGGSLRLSCVAS | 5.845 | | |
| 112-2- | | GRTFSRWHTGWYRQAPGREREFVAT | | | |
| D08 | | LRASGGDTYYADSVKGRFTISRDNA | | | |
| | | KNTVYLQMNSLKPEDTAVYYCNVW | | | |
| | | ANWGAPPSDFSSWGQGTQVTVSS | | | |
| SDAB22 | 70 | QLQLVESGGGLVQPGGSLRLSCAAS | 9.401 | | |
| 112-1- | | GFTFSSYSMSWYRQAPGKERELVATI | | | |
| C10 | | DTDGRTNYADSVKGRFTISRDNAKN | | | |
| | | SVYLQMNSLKPEDTAVYYCNRRQLG | | | |
| | | VDYWGQGTQVTVSS | | | |
| SDAB22 | 71 | QVQLVESGGGLVQPGGSLRLSCAAS | 10.39 | | |
| 112-1- | | GFTFSNYGMSWVRQAPGKGLEWVS | | | |
| D12-3 | | DVNSSGSRRFYVDSVKGRFTISRDNA | | | |
| | | KNTVFLQMNSLKPEDSAVYYCVKLA | | | |
| | | EAGTLIHVGSWGQGTRVTVSS | | | |
| SDAB22 | 72 | QLQLVESGGGLVQPGGSLRLSCVGS | 13.14 | | |
| 112-1- | | GFTFSLASMGWYRQAPGKEREWVA | | | |
| C9 | | SISSLDASTNYADSVKGRFTISRDNA | | | |
| | | KRMVYLQMNNLKSEDTAVYYCKA | | | |
| | | MNYWGKGTQVTVSS | | | |
| SDAB22 | 73 | QLQLVESGGGLVQPGGSLRLSCAAS | 8.418 | | |
| 112-1- | | RSIFSSYVMAWYRRAPGKKRELVASI | | | |
| B6-B05 | | ANVGSNTDYASFAKGRFTISRDDDKS | | | |
| | | RDNDKITVYLQMNSLNPEDTAVYYC | | | |
| | | NAWLGAGSDYWGQGTQVTVSS | | | |

### Example 6: Identification of Anti-can f2 VHH

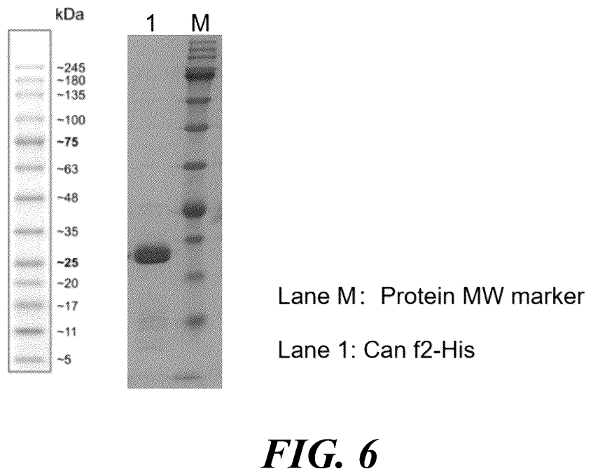

In order to obtain single variable domain on heavy chain, i.e., VHH, specific for dog allergen Can f2, a recombinant His-tagged Can f2 was prepared, which reached high purity, as shown via SDS-Page Western blotting in FIG. 6. To confirm this, an ELISA was performed measuring optical density when the recombinant Can f2-His is incubated with a biotinylated anti-His antibody. As summarized in Table 7, the recombinant Can f2-His was effectively bound by the antibody.

TABLE 7
Recombinant Can f2-His ELISA Binding Assay

| Can f2-His Concentration | Repeat 1 | Repeat 2 |
|---|---|---|
| (μg/mL) | (OD at 450 nm) | (OD at 450 nm) |
| 10 | 1.12 | 1.07 |
| 2 | 0.57 | 0.65 |
| 0.4 | 0.22 | 0.19 |
| 0.08 | 0.12 | 0.13 |
| 0.016 | 0.10 | 0.11 |
| 0.0032 | 0.11 | 0.11 |
| 0.00064 | 0.10 | 0.11 |
| Blank | 0.11 | 0.07 |

The recombinant Can f2 was then administered to two alpacas in four rounds. Alpacas were administered 250 ug of the recombinant Can f2 on day 0, day 14, day 28, and day 49. An ELISA using titrated serum from blood was performed detecting levels of anti-Can f2 VHH. The results of which are summarized in Table 8. This was also repeated with alpacas immunized with a sumomylated Can f2-His, the results of which are found in Table 9. Suomo tags were utilized to test its efficacy in increasing solubility. Each measurement was performed in duplicate. Negative serum was taken from naïve alpacas to be used as a negative control.

TABLE 8
Can f2-His Immunized Alpaca Serum Titration

| | | OD at 450 nm | OD at 450 nm |
|---|---|---|---|
| | | Day 14 (2^{nd} | Day 28 (3^{rd} |
| Titration | Negative Serum | Immunization) | Immunization) |

Alpaca 1

| | | | | | | |
|---|---|---|---|---|---|---|
| 1:1K | 0.128 | 0.133 | 2.171 | 1.809 | 2.466 | 2.207 |
| 1:2K | 0.093 | 0.099 | 1.737 | 1.522 | 2.156 | 1.893 |
| 1:4k | 0.077 | 0.075 | 1.460 | 1.375 | 1.723 | 1.774 |
| 1:8K | 0.076 | 0.076 | 1.233 | 1.245 | 1.681 | 1.559 |
| 1:16K | 0.075 | 0.076 | 0.783 | 0.809 | 1.220 | 1.245 |
| 1:32k | 0.069 | 0.062 | 0.562 | 0.530 | 0.874 | 0.891 |
| 1:64K | 0.064 | 0.069 | 0.359 | 0.340 | 0.626 | 0.584 |
| PBS | 0.083 | 0.088 | 0.096 | 0.092 | 0.084 | 0.111 |

Alpaca 2

| | | | | | | |
|---|---|---|---|---|---|---|
| 1:1K | 0.227 | 0.241 | 2.001 | 1.985 | 2.604 | 2.638 |
| 1:2K | 0.143 | 0.133 | 1.497 | 1.404 | 2.056 | 2.060 |
| 1:4k | 0.108 | 0.101 | 1.098 | 1.081 | 1.626 | 1.578 |
| 1:8K | 0.099 | 0.084 | 0.772 | 0.678 | 1.263 | 1.252 |
| 1:16K | 0.080 | 0.075 | 0.454 | 0.464 | 0.867 | 0.799 |
| 1:32k | 0.075 | 0.068 | 0.290 | 0.302 | 0.573 | 0.530 |
| 1:64K | 0.070 | 0.062 | 0.194 | 0.195 | 0.363 | 0.334 |
| PBS | 0.080 | 0.072 | 0.073 | 0.080 | 0.100 | 0.081 |

TABLE 9
Can f2-sumo-His Immunized Alpaca Serum Titration

| | | OD at 450 nm | OD at 450 nm |
|---|---|---|---|
| | | Day 14 (2^{nd} | Day 28 (3^{rd} |
| Titration | Negative Serum | Immunization) | Immunization) |

Alpaca 1

| | | | | | | |
|---|---|---|---|---|---|---|
| 1:1K | 0.119 | 0.111 | 4.028 | 4.074 | 4.120 | 4.205 |
| 1:2K | 0.088 | 0.085 | 3.755 | 3.792 | 3.855 | 4.047 |
| 1:4k | 0.071 | 0.072 | 3.438 | 3.392 | 3.828 | 3.973 |
| 1:8K | 0.072 | 0.072 | 2.976 | 2.909 | 3.690 | 3.817 |
| 1:16K | 0.071 | 0.071 | 2.190 | 2.215 | 3.212 | 3.487 |
| 1:32k | 0.056 | 0.057 | 1.560 | 1.557 | 2.570 | 2.883 |
| 1:64K | 0.057 | 0.058 | 1.011 | 0.999 | 1.923 | 2.109 |
| PBS | 0.078 | 0.080 | 0.088 | 0.079 | 0.078 | 0.114 |

Alpaca 2

| | | | | | | |
|---|---|---|---|---|---|---|
| 1:1K | 0.186 | 0.181 | 4.060 | 3.970 | 4.053 | 4.084 |
| 1:2K | 0.119 | 0.113 | 3.318 | 3.461 | 3.804 | 3.846 |
| 1:4k | 0.090 | 0.081 | 2.915 | 2.860 | 3.576 | 3.564 |
| 1:8K | 0.083 | 0.079 | 2.267 | 2.290 | 3.192 | 3.196 |
| 1:16K | 0.074 | 0.081 | 1.506 | 1.520 | 2.591 | 2.598 |
| 1:32k | 0.071 | 0.061 | 1.004 | 1.009 | 1.957 | 1.957 |
| 1:64K | 0.070 | 0.057 | 0.638 | 0.616 | 1.319 | 1.322 |
| PBS | 0.084 | 0.067 | 0.064 | 0.068 | 0.066 | 0.075 |

After the 3^{rd} immunization, peripheral blood mononuclear cells (PBMCs) were obtained from the immunized alpacas, RNA was extracted from the PBMCs, and RNA was reverse transcribed into cDNA. VHH sequences were then amplified from cDNA using the single domain antibody cloning primer combinations and subcloned into the yeast display vector pYDisplay, which were electro-transformed to EYB100 competent cells to construct single domain antibody yeast display libraries.

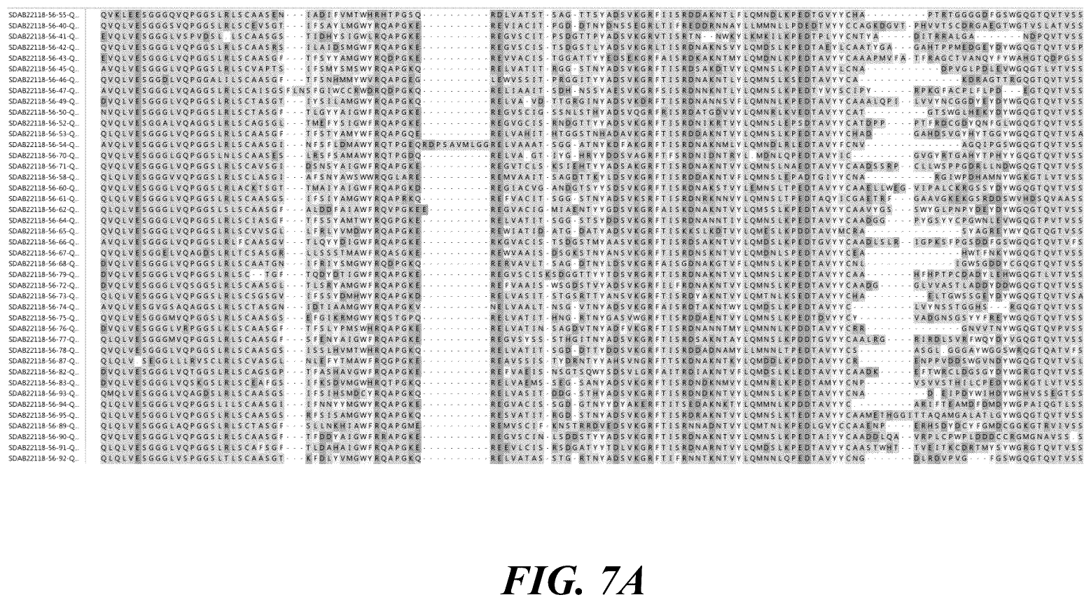

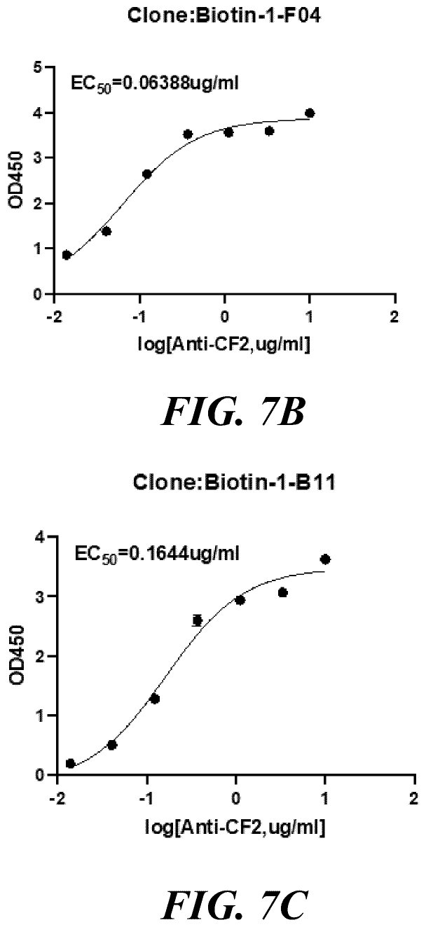

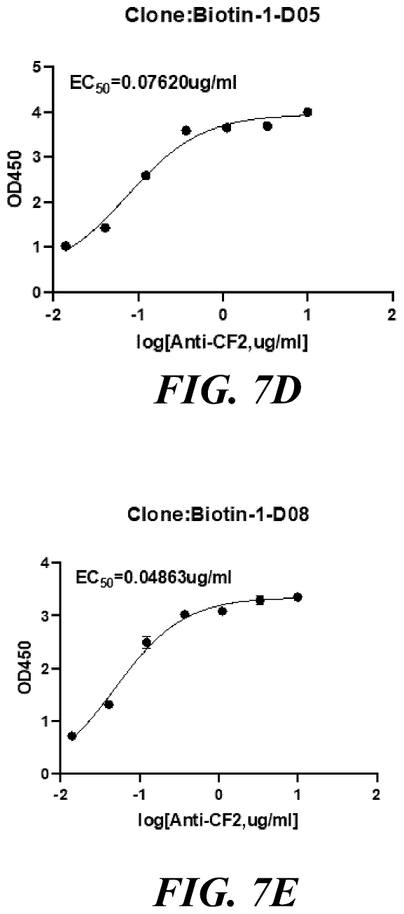

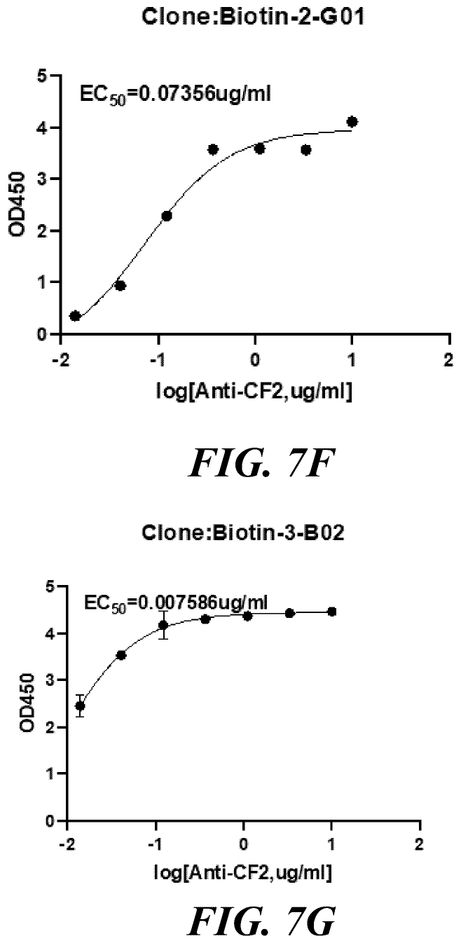

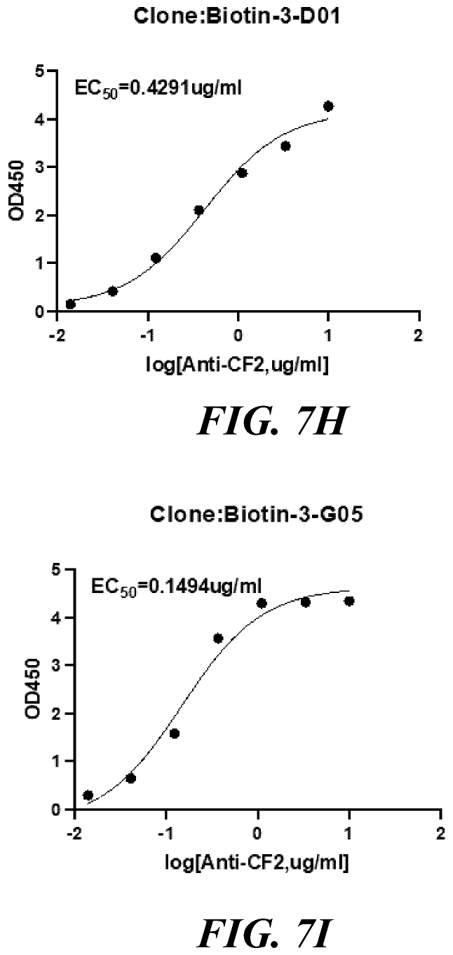

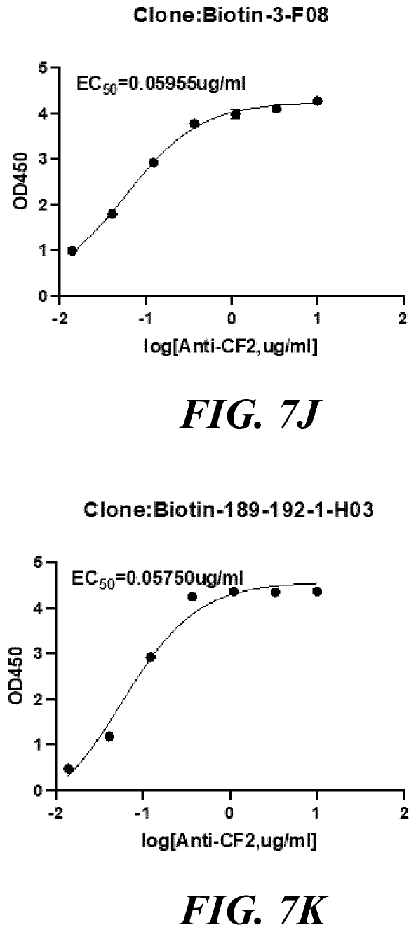

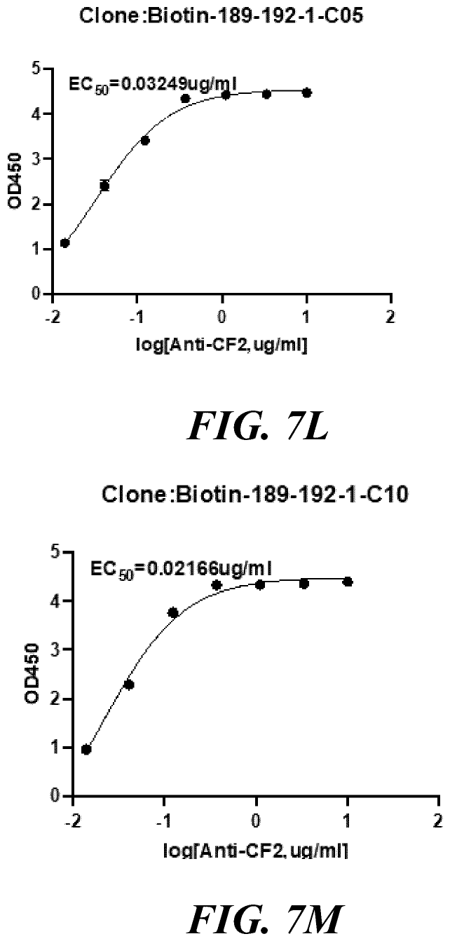

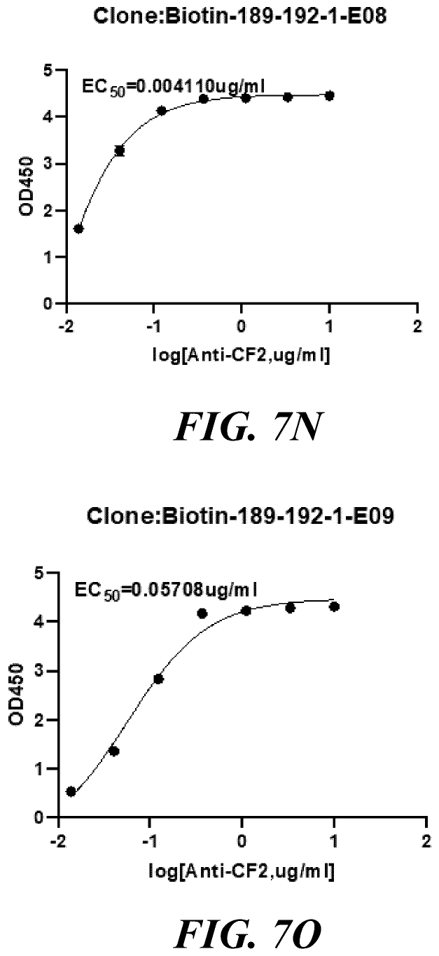

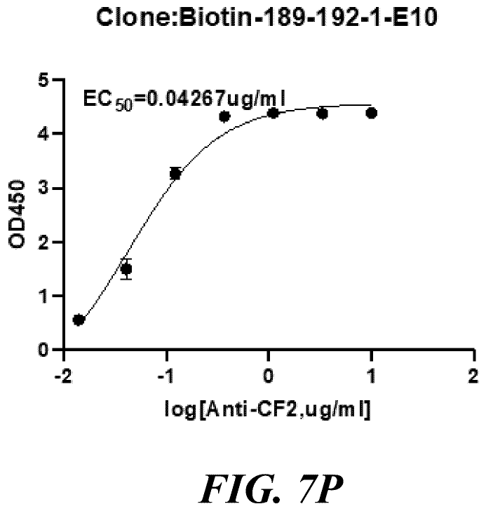

The sequences of 50 randomly selected clones are shown in FIG. 7A, which demonstrated low overlap, indicating a diverse library. Single clones were subjected to a flow cytometry-based assay in which biotinylated recombinant Can f2 was incubated with the VHH clone and following a wash step, detection of bound Can f2 identified the clone as a VHH candidate. VHH candidates were then constructed into prokaryotic expression vectors.

Isolated VHH clones were further validated by performing an ELISA assay as described in Example 1. Metrics of thermostability including melting temperature (Tₘ), onset temperature (Tₒₙₛₑₜ), and temperature of aggregation (T_{agg}), were also tested with increasing temperature in the range of 20° C.-95° C. at the speed of 1° C./min. Sequence information for the VHH clones and the binding assay results are summarized in Table 10. The individual binding curves are shown for each candidate in FIGS. 7B-7P. Each clone demonstrated strong binding, indicated by low EC₅₀ values.

TABLE 10
Anti-Can f2 VHH Validation

| | | | EC₅₀ | | |
|---|---|---|---|---|---|
| | SEQ | | (μg/ | | |
| Clone | ID NO: | Sequence | mL) | T_{agg} | Tₘ |
| SDAB22 | 9 | QVQLVESGGGLVQPGGSLRLSCAYS | 0.06388 | 48.08 | 58.63 |
| 118-1- | | GFTLDNNVIGWFRQAPGKEREFVAAI | | | |
| F04 | | SRSGAFTHYTESVQGRFTISRDNAKN | | | |
| | | TVYLQMNSLKPEDTAVYYCAAGAIL | | | |
| | | LPTERRYDYWGQGTQVTVSS | | | |
| SDAB22 | 10 | AVQLVESGGGLVQPGGSLRLSCAAS | 0.1644 | 44.54 | 55.17 |
| 118-1- | | GFTFSNYYMSWYRQAPGKEREWVA | | | |
| B11 | | FITSTGSSTNYANSVKGRFTASRDNA | | | |
| | | KNTVYLQMNSLKPEDTAVYYCKTRL | | | |
| | | WGNDYWGKGTLVTVSS | | | |
| SDAB22 | 11 | QVQLVESGGGLVQAGGSLGLSCAAS | 0.0762 | 39.54 | Tm1: |
| 118-1- | | GRTFNNYVMGWFRQAPGKEREFVA | | | 48.35 |
| D05 | | AISRSGSFTHYAEAVQGRFTISRDNA | | | Tm2: |
| | | KITVYLQMNSLKPEDTAVYYCAAGA | | | 58.58 |
| | | ILMPTERTYDYWGQGTQVTVSS | | | Tm3: |
| | | | | | 64.58 |
| SDAB22 | 12 | EVQLVESGGGLVQPGGSLRLSCVVS | 0.04863 | 40.82 | Tm1: |
| 118-1- | | GSIFSDNAMGWYRQAPGKQREMVAI | | | 38.89 |
| D08 | | ISSVGTTNNVDSVNGRFTISRDNAKN | | | Tm2: |
| | | TVYLQMNSLKPEDTAVYYCKDFSAP | | | 43.62 |
| | | RYWGQGTQVTVSS | | | Tm3: |
| | | | | | 54.69 |
| SDAB22 | 13 | AVQLVDSGGGLVQAGDSLRLSCVAS | 0.07356 | 38.93 | 43.71 |
| 118-2- | | GRTFSSYVMGWFRQAPGKERLIVATI | | | |
| G01 | | SKSGSLTHYADSVEGRFTISRDNAKN | | | |
| | | TVYLQMNSLEPEDTAVYYCTPVSDL | | | |
| | | TGRRLGSYWGQGTQVTVSS | | | |
| SDAB22 | 14 | AVQLVDSGGGLVQPGGSLRLSCAAS | 0.007586 | | |
| 118-3- | | GRTFSYYAMGWFRQALGKEREFVA | | | |
| B02 | | ASSRTGRVTNYADSVKGRFTISRDNA | | | |
| | | KNTVYLQMNSLKPEDTAIYYCAADD | | | |
| | | RFYGGDNPAFYNSWGQGTQVTVSS | | | |
| SDAB22 | 15 | QVQLVESGGGLVQAGDSLRLSCAAS | 0.4291 | | |
| 118-3- | | GRSFSNYVMNWFRQAPGKEREFVAA | | | |
| D01 | | ISRSGRSTFYADSVKGRFTISRDNPKA | | | |
| | | TVYLQMNSLVIEDTAVYFCAAASDIT | | | |
| | | SMRSLAAITSWGQGTQVIVSS | | | |
| SDAB22 | 16 | AVQLVESGGGLVQAGGSLRLSCAAS | 0.1494 | | |
| 118-3- | | GRTFNNSVMGWFRQAPGKEREFVA | | | |
| G05 | | AISRSGAFAHYAESVEGRFTISRDNA | | | |
| | | KNTVYLQMNSLKPEDTAVYYCAAG | | | |
| | | AILLPTERRYDYWGQGTQVTVSS | | | |
| SDAB22 | 17 | QVQLVESGGGLVQPGESLRLSCAAS | 0.05955 | | |
| 118-3- | | GSIFSIYRMGWYRQAPGEQREHVAT | | | |
| F08 | | VTSGGGTGYADSVKGRFTIYRDNAK | | | |
| | | NTVYLQMNSLKPEDTAVYSCYAIPK | | | |
| | | SWSRTSEYSWGQGTQVTVSS | | | |
| SDAB22 | 18 | EVQLVESGGGLVQARGSLRLSCAAS | 0.0575 | | |
| 118-189- | | GRTFNNYAMGWFRQAPGKEREFVA | | | |
| 192-1- | | AIASNTGTTYYAGSVKGRFAISRDNA | | | |
| H03 | | KNTVDLHMNSLKPEDTAVYYCALGP | | | |
| | | LRLGWWYDSRPYDYWGQGTQVTVS | | | |
| | | S | | | |
| SDAB22 | 19 | QVQLVESGGGGVQAGGSLRLSCVAS | 0.03249 | | |
| 118-189- | | QSHFGYNVMGWYRQAPGRQRELVA | | | |
| 192-1- | | TIISSGGTNYADSVKGRFTISRDNAKN | | | |
| C05 | | TVHLSMDSLNVEDTAVYFCYAKGV | | | |
| | | WLGREYWGQGTQVTVSS | | | |
| SDAB22 | 20 | EVQLVESGGGLVQAGGSLRLSCAAS | 0.02166 | | |
| 118-189- | | GRTFSEYIMGWFRQAPGKERVFVSTI | | | |
| 192-1- | | SKSGAITNYADSVQGRFTISRDNAKN | | | |
| C10 | | TVYLQMNSLKPEDSAVYYCAAGPLL | | | |
| | | SPAGRQYDYWGQGTQVTVSS | | | |
| SDAB22 | 21 | AVQLVESGGGLVQPGGSLRLSCVAS | 0.00411 | | |
| 118-189- | | GSSSRNYAMGWYRQAPGNEREFVA | | | |
| 192-1- | | VITSRGYTHYANSVSGRFTISRDHAK | | | |
| E08 | | DTAYLQMNSLKPEDTAVYYCNTQA | | | |
| | | GLLPFMTVYDWGTGTQVTVSS | | | |
| SDAB22 | 22 | EVQLVESGGGLVQPGGSLRLSCAAS | 0.05708 | | |
| 118-189- | | GTIFLINRMGWYRQAPGKQRELVAT | | | |
| 192-1- | | SFTSGNSTMYADSVKGRFTISRDNAK | | | |
| E09 | | KTVYLQMNSLKPEDTAVYYCKARIS | | | |
| | | RRYGNWDDYWGQGTQVTVSS | | | |
| SDAB22 | 23 | AVQLVDSGGGLVQTGGSLRLSCAAS | 0.04267 | | |
| 118-189- | | GTIFSINRMAWYRQAPGKQRELVASI | | | |
| 192-1- | | FFSGGITHYADFVKGRFTISRDNAKN | | | |
| E10 | | TMYLQMNSLKPEDTAVYYCKGVIAP | | | |
| | | NYRRTSNFQDYWGQGTQVTVSS | | | |

### Example 7: Identification of Anti-Der p1 VHH

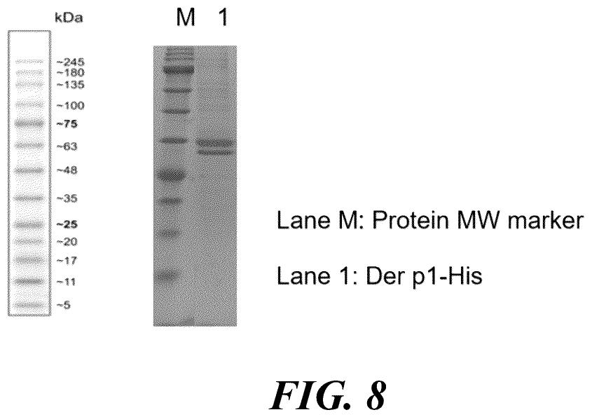

In order to obtain single variable domain on heavy chain, i.e., VHH, specific for dust mite allergen Der p1, a recombinant His-tagged Der p1 was prepared, which reached high purity, as shown via SDS-Page Western blotting in FIG. 8. To confirm this, an ELISA was performed measuring optical density when the recombinant Der p1-His is incubated with a known Der p1 antibody (clone 10B9). As summarized in Table 11, the recombinant Der p1-His was effectively bound by the antibody.

TABLE 11
Recombinant Der p1-His ELISA Binding Assay

| Der p1-His Concentration | Repeat 1 | Repeat 2 |
|---|---|---|
| (μg/mL) | (OD at 450 nm) | (OD at 450 nm) |
| 10 | 4.37 | 4.40 |
| 2 | 4.03 | 4.31 |
| 0.4 | 1.80 | 1.87 |
| 0.08 | 0.58 | 0.49 |
| 0.016 | 0.20 | 0.17 |
| 0.0032 | 0.25 | 0.12 |
| 0.00064 | 0.13 | 0.12 |
| Blank | 0.13 | 0.11 |

The recombinant Der p1 was then administered to two alpacas in four rounds. Alpacas were administered 250 ug of the recombinant Der p1 on day 0, day 14, day 28, and day 49. A fifth round of immunization was completed on one of the alpacas on Day 56. An ELISA using titrated serum from blood was performed detecting levels of anti-Der p1 VHH. The results of which are summarized in Table 12. Each measurement was performed in duplicate. Negative serum was taken from naïve alpacas to be used as a negative control.

TABLE 12
Der p1 Immunized Alpaca Serum Titration
Alpaca 1

| | | | OD at 450 nm | | OD at 450 nm | |
|---|---|---|---|---|---|---|
| | | | Day 28 (3^{rd} | | Day 49 (4^{th} | |
| Titration | Negative Serum | | Immunization) | | Immunization) | |
| 1:1K | 0.216 | 0.191 | 2.770 | 3.005 | 4.021 | 3.960 |
| 1:2K | 0.145 | 0.124 | 1.847 | 2.003 | 3.407 | 3.369 |
| 1:4k | 0.105 | 0.089 | 1.178 | 1.268 | 2.625 | 2.613 |
| 1:8K | 0.100 | 0.083 | 0.757 | 0.756 | 1.711 | 1.695 |
| 1:16K | 0.085 | 0.073 | 0.447 | 0.460 | 1.118 | 1.091 |
| 1:32k | 0.094 | 0.073 | 0.265 | 0.278 | 0.722 | 0.745 |
| 1:64K | 0.082 | 0.061 | 0.163 | 0.133 | 0.455 | 0.470 |
| PBS | 0.083 | 0.079 | 0.078 | 0.065 | 0.062 | 0.065 |

Alpaca 2

| | | | OD at 450 nm | | OD at 450 nm | |
|---|---|---|---|---|---|---|
| | | | Day 49 (4^{th} | | Day 56 (5^{th} | |
| Titration | Negative Serum | | Immunization) | | Immunization) | |
| 1:1K | 0.387 | 0.399 | 3.119 | 3.021 | 4.140 | 4.070 |
| 1:2K | 0.251 | 0.284 | 1.968 | 2.048 | 3.313 | 3.319 |
| 1:4k | 0.185 | 0.218 | 1.091 | 1.159 | 2.115 | 2.128 |
| 1:8K | 0.135 | 0.159 | 0.652 | 0.719 | 1.309 | 1.275 |
| 1:16K | 0.104 | 0.123 | 0.385 | 0.394 | 0.775 | 0.772 |
| 1:32k | 0.104 | 0.103 | 0.244 | 0.243 | 0.480 | 0.485 |
| 1:64K | 0.097 | 0.097 | 0.119 | 0.110 | 0.350 | 0.335 |
| PBS | 0.089 | 0.084 | 0.110 | 0.111 | 0.112 | 0.108 |

After the 4th and 5th immunization, peripheral blood mononuclear cells (PBMCs) were obtained from the two immunized alpacas, respectively. Then, RNA was extracted from the PBMCs, and RNA was reverse transcribed into cDNA. VHH sequences were then amplified from cDNA using the single domain antibody cloning primer combinations and subcloned into the yeast display vector pYDisplay, which were electro-transformed to EYB100 competent cells to construct single domain antibody yeast display libraries.

The sequences of 50 randomly selected clones demonstrated low overlap, indicating a diverse library. Single clones were subjected to a flow cytometry-based assay in which biotinylated recombinant Der p1 was incubated with the VHH clone and following a wash step, detection of bound Der p1 identified the clone as a VHH candidate. VHH candidates were then constructed into prokaryotic expression vectors.

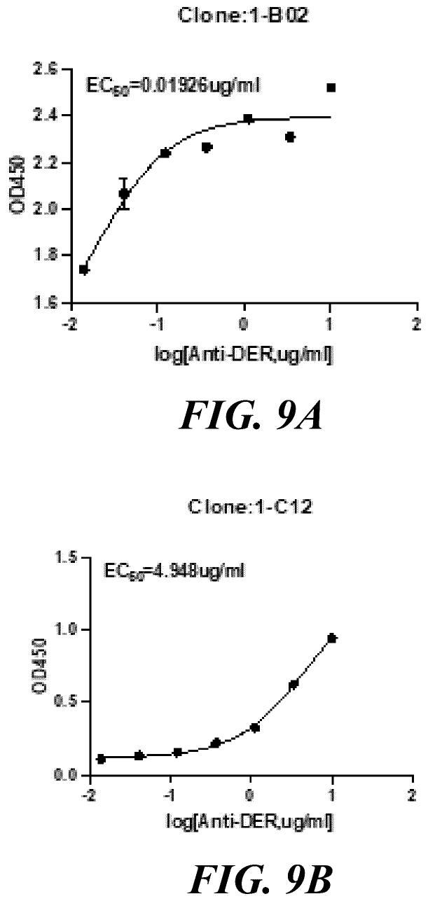

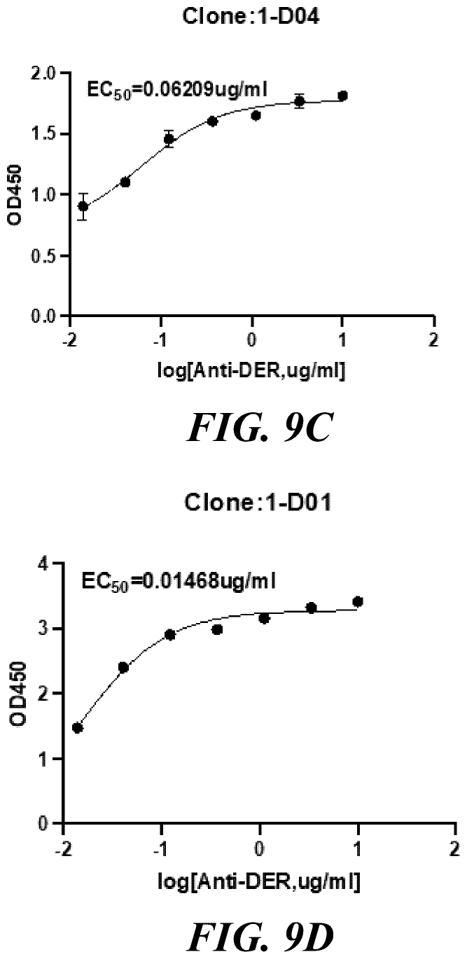

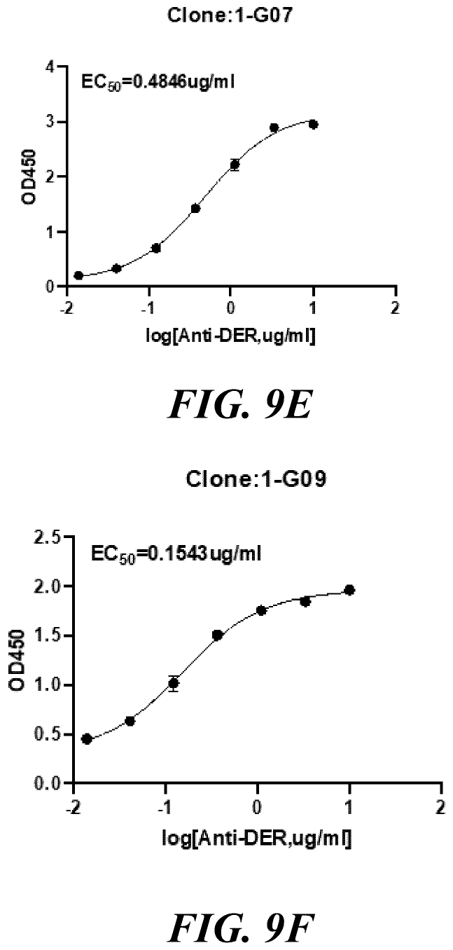

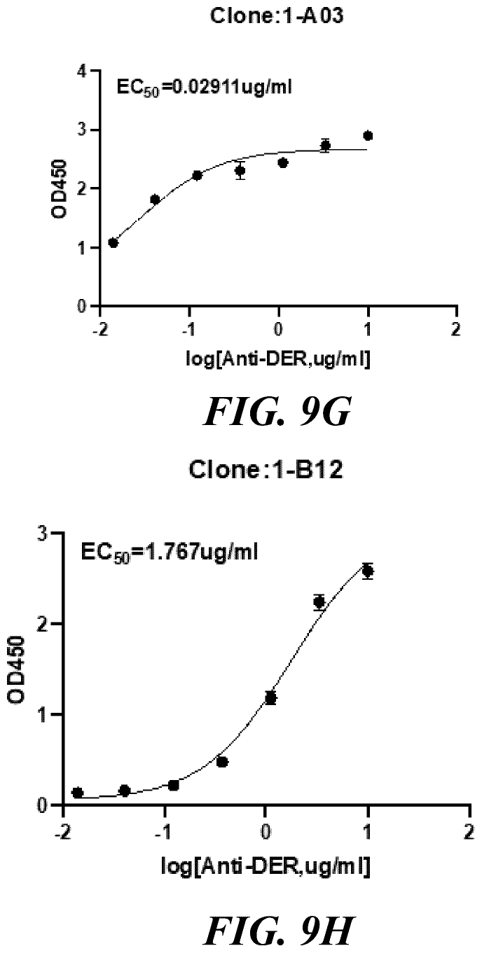

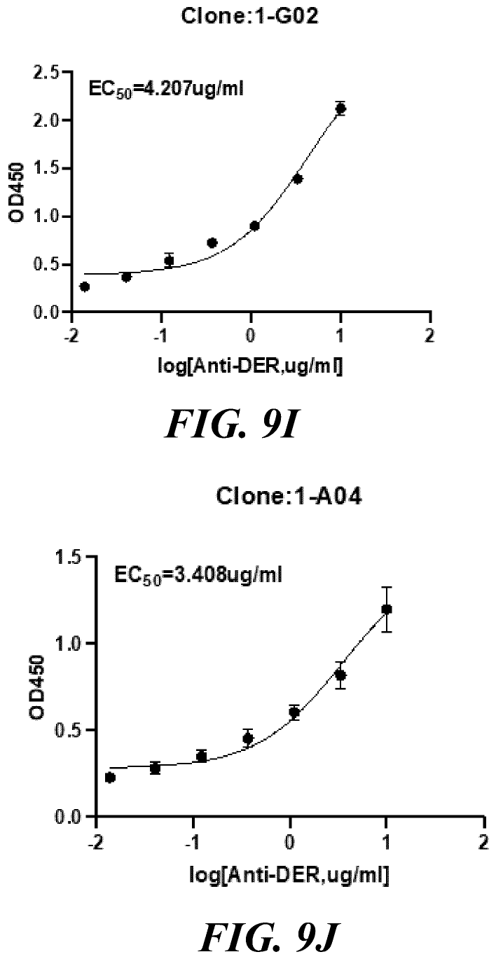

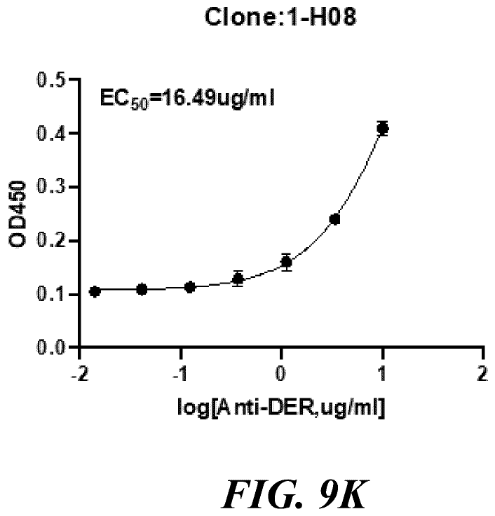

Isolated VHH clones were further validated by performing an ELISA assay as described in Example 1. Sequence information for the VHH clones and the binding assay results are summarized in Table 13. The individual binding curves are shown for each candidate in FIGS. 9A-9K. Each clone demonstrated strong binding, indicated by low EC50 values.

TABLE 13
Anti-Der p1 VHH Validation

| | | | EC₅₀ | | |
|---|---|---|---|---|---|
| | SEQ | | (μg/ | | |
| Clone | ID NO: | Sequence | mL) | T_{agg} | Tₘ |
| SDAB22 | 24 | QLQLVESGGGLVQSGGSLRLSCVAK | 0.01926 | | |
| 129-55- | | GGTNHPYPIGWFRQAPGKEQEGVLCI | | | |
| 35-46- | | ASGGQTQDSAHNPNWADSVKGRFTI | | | |
| 49-1-B02 | | SRDDATHTVYLEMNNLKADDTAVY | | | |
| | | FCAAPREIYIDSRCATYKYEYWGQGT | | | |
| | | QVTVSP | | | |
| SDAB22 | 25 | QVQLVESGGGLVQAGGSLRLSCAAS | 4.948 | | |
| 129-55- | | GRTFSSYAMGWFRQAPGKEREFVAII | | | |
| 35-46- | | SNTGGLTDYAHSVKGRFTISRDNAK | | | |
| 49-1-C12 | | NTGYLQMNSLKPEDTALYYCAADFL | | | |
| | | GPKSWPSYWGQGTQVTVSS | | | |
| SDAB22 | 26 | QLQLVESGGGLVQPGGSLRLSCVISG | 0.06209 | | |
| 129-55- | | GTFHPYPIGWFREAPGKEREGVLCIN | | | |
| 35-46- | | SGGQSEISANSAHDPKYADSVKGRFT | | | |
| 49-1-D04 | | ISRDNAANTVYLQMNNLEPGDTAVY | | | |
| | | YCAAPREIYVDPYCPTYAYEYWGQG | | | |
| | | TQVTVSA | | | |
| SDAB22 | 27 | QVQLVESGGGLVQAGGSLRLSCAAS | 0.01468 | | |
| 129-55- | | GRTFSRYAMGWFRQAPGKEREFVAA | | | |
| 35-46- | | ITGSGSRTYYADSIQGRFTISRDNAKN | | | |
| 49-1-D01 | | TVYLQMNSLKPEDTAVYYCVQGWA | | | |
| | | EATMTSLGEDYDYWGQGTQVTVSS | | | |
| SDAB22 | 28 | QVQLVESGGGLVQAGGSLRLSCAAS | 0.4846 | | |
| 129-55- | | GRTFSSYAMGWFRQAPGKERELVAT | | | |
| 35-46- | | ISWSGGSTYYADSVKGRFTISRDNAK | | | |
| 49-1-G07 | | NTGFLQMNSLKPEDTAVYYCAADFA | | | |
| | | STVGTPLTRPAYWGQGTQVTVSS | | | |
| SDAB22 | 29 | QVKLEESGGGAVQPGGSLRLSCTAS | 0.1543 | | |
| 129-55- | | GQTFSNYIISWFRQAPGKEREFVVGIS | | | |
| 35-46- | | KSGGRTYYADSAKGRFTISRDNAKN | | | |
| 49-1-G09 | | TVYLQMSSLKPEDTAVYYCAADGLV | | | |
| | | LTAAAAEYDYWGQGTQVTVSS | | | |
| SDAB22 | 30 | QVQLVESGGGLVQAGGSLRLSCAVS | 0.02911 | | |
| 129-55- | | GRTVSRNVMGWFRQAPGKEREFVA | | | |
| 35-46- | | GIGFSGGSTYYADSVKGRFTISRDNA | | | |
| 49-1-A03 | | KNTVYLEMNRLQPEDTAVYYCAAPS | | | |
| | | LPLLTSDLHDYDYWGQGTQVTVSS | | | |
| SDAB22 | 31 | QVQLVESGGGLVQAGGPLRLSCAAS | 1.767 | | |
| 129-55- | | GRTFSSYAMGWFRQAPGKEREFVAA | | | |
| 35-46- | | ISWSGDSTYYADSVKGRFTLSRDNA | | | |
| 49-1-B12 | | KNTVYLQMNSLKPEDTAVYYCAVPS | | | |
| | | SGRGTYYYTLSAYEYWGQGTQVTVF | | | |
| | | S | | | |
| SDAB22 | 32 | QLQLVESGGGLVQAGGSLRLSCTAS | 4.207 | | |
| 129-55- | | GRTFRNFGMGWFRQAPGKERKLVAS | | | |
| 35-46- | | ISYVGGNTDYADSVKGRFTISMDNA | | | |
| 49-1-G02 | | KNTVVLQMNSLKPEDTAMYYCAAR | | | |
| | | NPNRNEYPWWGQGTQVTVSS | | | |
| SDAB22 | 33 | QLQLVESGGGLVQPGGSLRLSCAAS | 3.408 | | |
| 129-55- | | GRVYNSWTMAWFRQAPGKEREFVG | | | |
| 35-46- | | AFSLLDSGTRYADSLKDRVAISRDDA | | | |
| 49-1-A04 | | ANTQWLQLSALKPEDTAVYYCAAKS | | | |
| | | GTIRATSEGQYNYWGQGIQVTVSS | | | |
| SDAB22 | 34 | EVQLVESGGGLVQAGGSLTLSCGRT | 16.49 | | |
| 129-55- | | TSIWGMGWFRQGRGKEREFVAAITP | | | |
| 35-46- | | SGSITFYSDHVKGRFTVSRDNALNTV | | | |
| 49-1-H08 | | YLQMNSLKPEDTAVYYCARRGSSGS | | | |
| | | YYWAGSYEAWGQGTQVTVSS | | | |

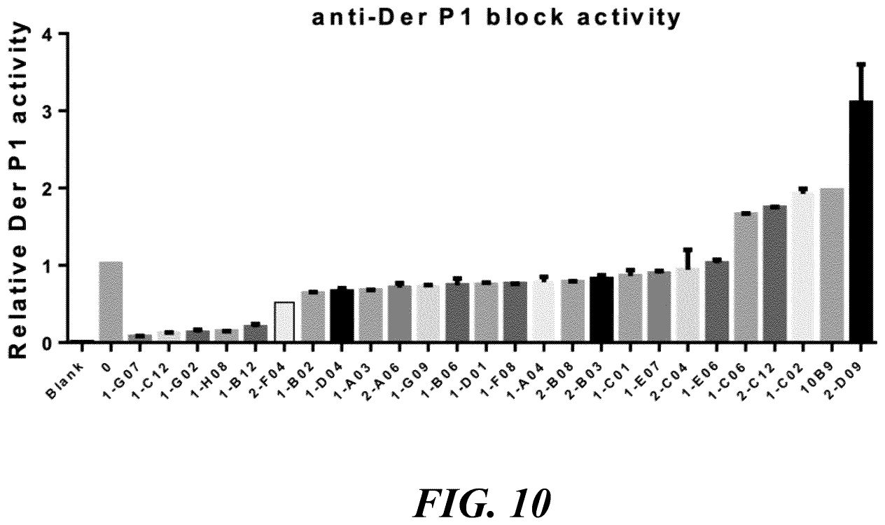

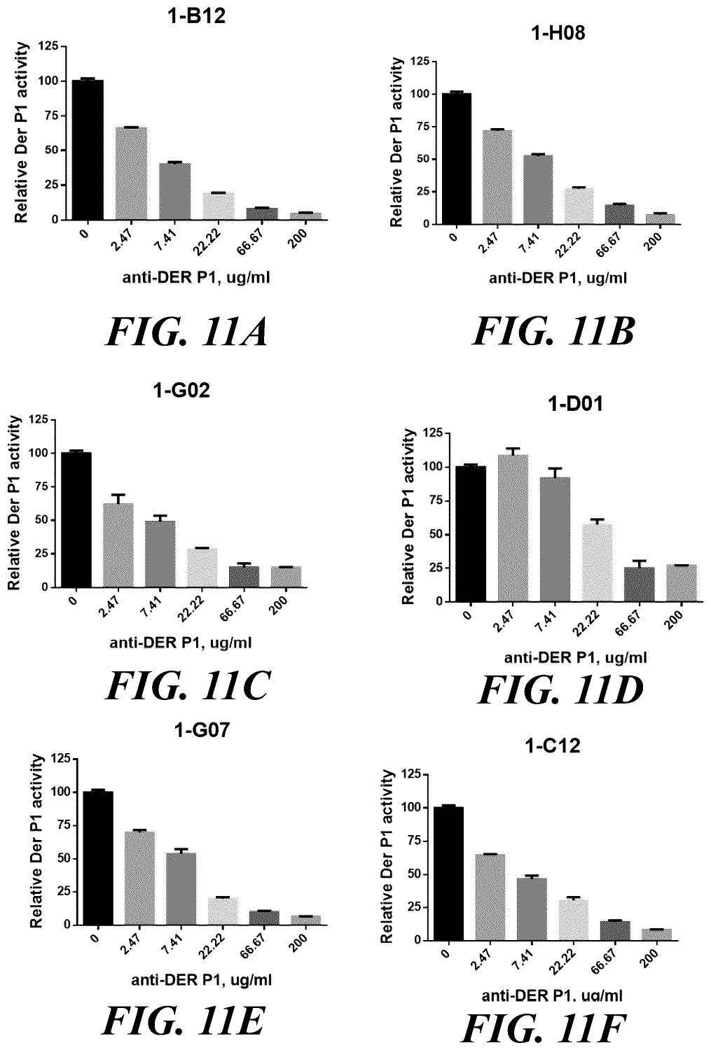

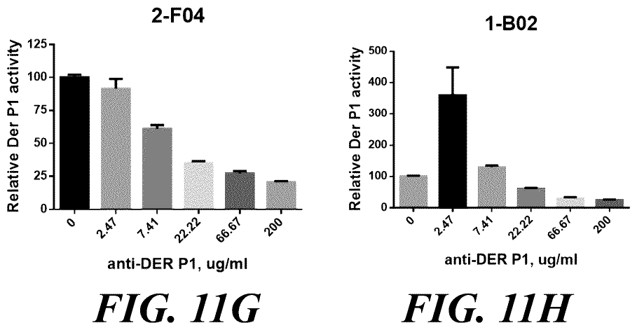

The cysteine protease activity of Der p1 is a major contributor to its allergenicity through enhancement of total IgE and Der p1-speciic IgE synthesis1. To screen for the VHH candidates that can block this enzymatic activity and thus the induced IgE synthesis, VHH candidates were incubated with Der p1 and cysteine solution, and the cysteine protease activity of Der p1 was measured in a continuous rate assay with the fluorogenic substrate. Der p1 was pre-activated with 5 mM cysteine (Sigma Chemical Co.) to regenerate its thiol group, which becomes oxidized during purification. The catalytic activity of Derp 1 was measured in a continuous rate (kinetic) assay using the fluorogenic peptide substrate N-tert-butoxy-carbonyl (Boc)-Gln-Ala-Arg-7-amino-4-methyl-coumarin (AMC), which was conducted in 50 mM sodium phosphate buffer, pH 7.0, containing 2.5 mM EDTA and 2.5 mM dithiothreitol (DTT) at 37° C. in a total volume of 1 ml. Hydrolysis of AMC substrates was monitored using a Hitachi F-2000 fluorescence with λex=380 nm and λem=460 nm. As summarized in FIG. 10 many VHH demonstrated blocking effects on Der p1 enzymatic activity. These candidates were further tested in titration experiments to confirm their activity. Each VHH was diluted in three folds by five times in sodium phosphate buffer starting at 0.6 mg/ml as shown in Table 14. Their activity of blocking Der p1 cysteine protease activity was measured. The results of the titration for each VHH is shown in FIGS. 11A-11H and summarized in Table 15.

TABLE 14
Titration Concentrations

| | | | | | |
|---|---|---|---|---|---|
| Antibody candidate | 600.00 | 200.00 | 66.67 | 22.22 | 7.41 |
| concentration (ug/ml) | | | | | |
| Antibody candidate final | 200.00 | 66.67 | 22.22 | 7.41 | 2.47 |
| concentration (ug/ml) | | | | | |

TABLE 15
Blocking Effect of VHH on Der pl Activity

| Clone | Maximum Rate of Blocking |
|---|---|
| 1-B12 | 95.38 |
| 1-G07 | 93.34 |
| 1-H08 | 92.83 |
| 1-C12 | 91.59 |
| 1-G02 | 85.17 |
| 2-F04 | 79.36 |
| 1-B02 | 75.36 |
| 1-D01 | 72.95 |

### Example 8. Identification of Der p1 Binding Peptides

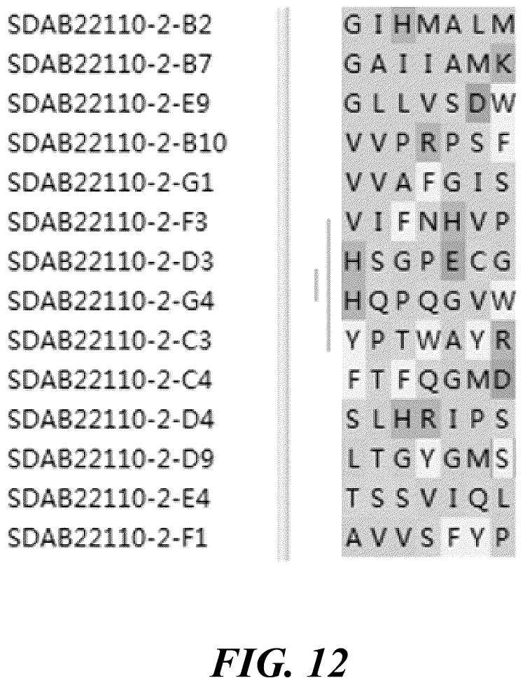

The recombinant Der p1 obtained in Example 7 was utilized to generate a phage display library to screen for 7-amino acid peptides that can bind to Der p1. This was performed similar to the display libraries described in the foregoing examples, using nucleic acids encoding the peptides which are expressed in phages. Shown in FIG. 12, a number of peptides were identified and demonstrated a high degree of diversity. The sequences of these peptides are summarized in Table 16.

TABLE 16
Der p1 Binding Peptides

| | SEQ | |
|---|---|---|
| Name | ID NO: | Sequence |
| SDAB22 | 35 | GIHMALM |
| 110-2-B2 | | |
| SDAB22 | 36 | GAIIAMK |
| 110-2-B7 | | |
| SDAB22 | 37 | GLLVSDW |
| 110-2-E9 | | |
| SDAB22 | 38 | VVPRPSF |
| 110-2- | | |
| B10 | | |
| SDAB22 | 39 | VVAFGIS |
| 110-2-G1 | | |
| SDAB22 | 40 | VIFNHVP |
| 110-2-F3 | | |
| SDAB22 | 41 | HSGPECG |
| 110-2-D3 | | |
| SDAB22 | 42 | HQPQGVW |
| 110-2-G4 | | |
| SDAB22 | 43 | YPTWAYR |
| 110-2-C3 | | |
| SDAB22 | 44 | FTFQGMD |
| 110-2-C4 | | |
| SDAB22 | 45 | SLHRIPS |
| 110-2-D4 | | |
| SDAB22 | 46 | LTGYGMS |
| 110-2-D9 | | |
| SDAB22 | 47 | TSSVIQL |
| 110-2-E4 | | |
| SDAB22 | 48 | AVVSFYP |
| 110-2-F1 | | |

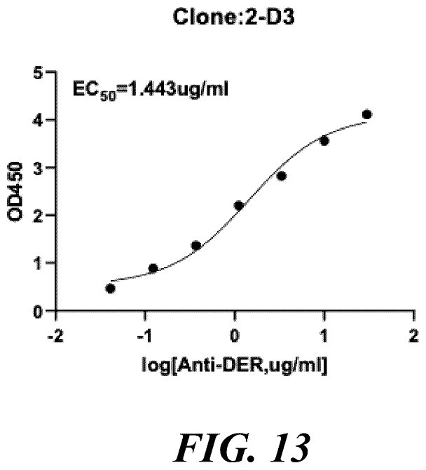

An ELISA was used to validate identified peptides. Der p1 was bound to the bottom of a plate. Different concentrations of the chosen (biotinylated) peptides were added and then washed. Then, HRP-streptavidin (which binds to the biotin) was added and an OD at 450 nm is measured. The results are summarized in Table 17. Duplicate measurements are separated by commas. An exemplary binding curve for peptide 2-D3 is shown in FIG. 13.

TABLE 17
Der p1 Peptide Binding Assay

| | Peptide: | Peptide: | Peptide: | Peptide: | Peptide: | Peptide: |
|---|---|---|---|---|---|---|
| Concentration | 2-B2 | 2-C3 | 2-D3 | 2-D9 | 2-F1 | 2-G1 |
| of Peptide | (OD450) | (OD450) | (OD450) | (OD450) | (OD450) | (OD450) |
| 30 | 0.536, | 0.684, | 4.097, | 0.751, | 0.938, | 0.666, |
| | 0.537 | 0.748 | 4.131 | 0.771 | 0.805 | 0.629 |
| 10 | 0.275, | 0.346, | 3.550, | 0.374, | 0.479, | 0.323, |
| | 0.289 | 0.376 | 3.571 | 0.357 | 0.516 | 0.293 |
| 3.333 | 0.132, | 0.162, | 2.770, | 0.154, | 0.221, | 0.147, |
| | 0.136 | 0.191 | 2.875 | 0.154 | 0.209 | 0.145 |
| 1.111 | 0.089, | 0.104, | 2.158, | 0.095, | 0.131, | 0.102, |
| | 0.089 | 0.102 | 2.250 | 0.102 | 0.125 | 0.109 |
| 0.370 | 0.083, | 0.072, | 1.372, | 0.068, | 0.076, | 0.088, |
| | 0.071 | 0.066 | 1.358 | 0.078 | 0.073 | 0.082 |
| 0.123 | 0.078, | 0.065, | 0.866, | 0.067, | 0.073, | 0.082, |
| | 0.066 | 0.065 | 0.906 | 0.068 | 0.075 | 0.073 |
| 0.041 | 0.070, | 0.058, | 0.468, | 0.061, | 0.079, | 0.057, |
| | 0.061 | 0.057 | 0.463 | 0.059 | 0.066 | 0.062 |
| 0.000 | 0.074, | 0.066, | 0.063, | 0.062, | 0.062, | 0.070, |
| | 0.071 | 0.063 | 0.067 | 0.067 | 0.070 | 0.065 |

### Example 9: Identification of Anti-Fel D1 VHH

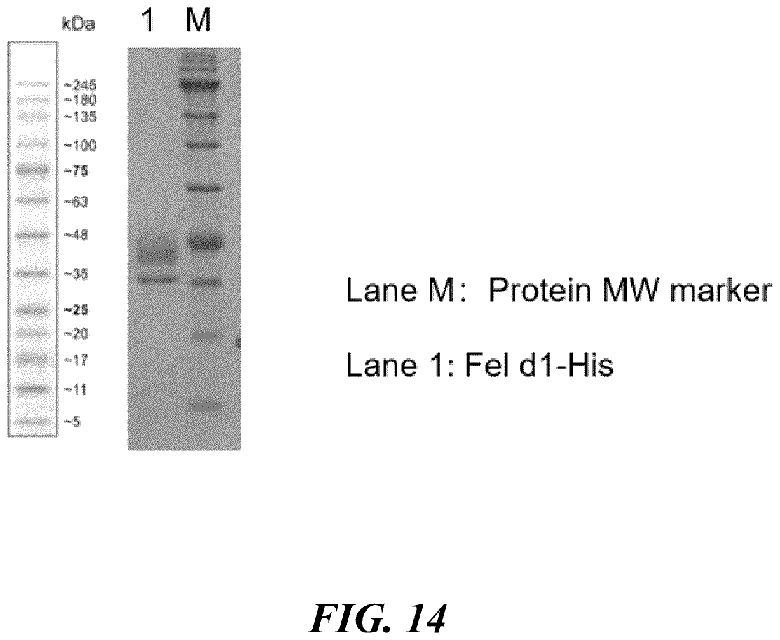

In order to obtain single variable domain on heavy chain, i.e., VHH, specific for cat allergen Fel D1, a recombinant His-tagged Fel D1 was prepared, which reached high purity, as shown via SDS-Page Western blotting in FIG. 14. To confirm this, an ELISA was performed measuring optical density when the recombinant Fel d1-His is incubated with two known Fel D1 antibodies which are summarized in Table 18. As summarized in Table 19, the recombinant Fel d1-His was effectively bound by both antibodies.

TABLE 18
Fel D1 Positive Control Antibodies

| | SEQ | | |
|---|---|---|---|
| | ID | | |
| Name | NO: | Name | Sequence |
| REGN1909 | 62 | Heavy Chain | EVQLVESGGGLVQPGGSLRLSCA |
| | | Variable | ASGFTFSSYAMSWVRQAPGKGLE |
| | | Region | WVSAISGRGYNADYADSVKGRFT |
| | | | ISRDNSKNTLYLQMNSLRAEDTA |
| | | | VYYCAKLEYFDYWGQGTLVTVS |
| | | | S |
| | 63 | Light Chain | DIQMTQSPSTLSASVGDRVTITCR |
| | | Variable | ASQSISSWLAWYQQKPGKAPKLL |
| | | Region | IYKASSLESGVPSRFSGSGSGTDFT |
| | | | LTISSLRPEDFATYYCQQYNSYPL |
| | | | TFGGGTKVEIK |
| 370 VH + | 64 | Heavy Chain | EVQLVESGGGLAQPGGSLRLSCA |
| 378 VL | | Variable | ASGFTFNNYAMTWVRQAPGKGL |
| | | Region | DWVSAISDSGRSTFSADSVKGRFT |
| | | | ISRDNSKNTLYLQMDSLRAEDTA |
| | | | LYYCAKHRNWNYPVFDYWGQG |
| | | | TLVTVSS |
| 370 VH + | 65 | Light Chain | DIQLTQSPSFLSASVGDRVTITCW |
| 378 VL | | Variable | ASQGISSYLAWYQQKPGKAPKLL |
| | | Region | IYSASTLQSGVPSRFSGSGSGTEFT |
| | | | LTISSLQPEDFATYYCQQLNSYPF |
| | | | TFGPGTKVDIK |
| 18 VH + | 66 | Heavy Chain | EVQLVESGGGLVKPGGSLRLSCA |
| 378 VL | | Variable | ASGFTFRNYNINWVRQAPGKGLE |
| | | Region | WVSLISGSSSYIYYADSVKGRFTV |
| | | | SRDNAKNSLYLQMNSLRAEDTA |
| | | | VYYCARRTLSYYVMDVWGQGTT |
| | | | VTVSS |
| 18 VH + | 65 | Light Chain | DIQLTQSPSFLSASVGDRVTITCW |
| 378 VL | | Variable | ASQGISSYLAWYQQKPGKAPKLL |
| | | Region | IYSASTLQSGVPSRFSGSGSGTEFT |
| | | | LTISSLQPEDFATYYCQQLNSYPF |
| | | | TFGPGTKVDIK |

TABLE 19
Recombinant Fel D1 His Binding Assay

| | |
|---|---|
| Volume of Fel D1-His (μL) | OD450 Using Positive Control: |
| (concentration ~1 μg/μL) | 18VH + 378VL |

| | | |
|---|---|---|
| 100 | 4.60 | 4.60 |
| 50 | 4.55 | 4.53 |
| 10 | 4.47 | 4.44 |
| Negative Control | 0.16 | 0.09 |

| | |
|---|---|
| | OD450 Using Positive Control: |
| Volume of Fel D1-His (μL) | 370VH + 378VL |

| | | |
|---|---|---|
| 100 | 4.60 | 4.60 |
| 50 | 4.55 | 4.53 |
| 10 | 4.47 | 4.44 |
| Negative Control | 0.16 | 0.09 |

Four distinct epitopes of Fel D1 were selected to be used for further identification. These epitopes are summarized in Table 20. Peptides were synthesized for the four epitopes for VHH identification.

TABLE 20
Fel D1 Epitopes Used

| SEQ | | | Positive |
|---|---|---|---|
| ID NO: | Name | Sequence | Control Ab |
| 58 | SDAB22110- | VAQYKALP | N/A |
| | 225-38AA | VVLENA | |
| 59 | SDAB22110- | FAVANGNE | REGN1909 |
| | 315-28AA-1 | LLLDLS | |
| 60 | SDAB22110- | AKMTEEDK | N/A |
| | 446-59AA | ENALS | |
| 61 | SDAB22110- | ENARILKN | 370 VH + 378 VL, |
| | 115-28AA-2 | CVDAKM | 18 VH + 378 VL |

The recombinant Fel D1 was then administered to two alpacas in four rounds. Alpacas were administered 250 ug of the recombinant Fel D1 on day 0, day 14, day 28, and day 49. A fifth round of immunization was completed on one of the alpacas on Day 56. An ELISA using titrated serum from blood was performed detecting levels of anti-Fel D1 VHH. The results of which are summarized in Table 21. Each measurement was performed in duplicate. Negative serum was taken from naïve alpacas to be used as a negative control.

TABLE 21
Fel D1 Immunized Alpaca Serum Titration
Alpaca 1

| | | | OD at 450 nm | | OD at 450 nm | |
|---|---|---|---|---|---|---|
| | | | Day 28 (3^{rd} | | Day 49 (4^{th} | |
| Titration | Negative Serum | | Immunization) | | Immunization) | |
| 1:1K | 0.258 | 0.289 | 3.438 | 3.300 | 4.137 | 3.971 |
| 1:2K | 0.167 | 0.176 | 2.630 | 2.550 | 3.858 | 3.806 |
| 1:4k | 0.132 | 0.138 | 1.793 | 1.702 | 3.654 | 3.648 |
| 1:8K | 0.128 | 0.115 | 1.139 | 1.065 | 3.046 | 3.033 |
| 1:16K | 0.103 | 0.103 | 0.727 | 0.668 | 2.319 | 2.266 |
| 1:32k | 0.106 | 0.115 | 0.447 | 0.416 | 1.625 | 1.612 |
| 1:64K | 0.107 | 0.108 | 0.278 | 0.257 | 1.018 | 1.041 |
| PBS | 0.135 | 0.112 | 0.107 | 0.098 | 0.112 | 0.103 |

Alpaca 2

| | | | OD at 450 nm | | OD at 450 nm | |
|---|---|---|---|---|---|---|
| | | | Day 49 (4^{th} | | Day 56 (5^{th} | |
| Titration | Negative Serum | | Immunization) | | Immunization) | |
| 1:1K | 0.248 | 0.220 | 3.705 | 3.586 | 3.957 | 4.115 |
| 1:2K | 0.151 | 0.155 | 2.904 | 2.741 | 3.859 | 3.971 |
| 1:4k | 0.120 | 0.119 | 2.131 | 2.040 | 3.808 | 4.009 |
| 1:8K | 0.114 | 0.117 | 1.434 | 1.330 | 3.768 | 3.917 |
| 1:16K | 0.096 | 0.098 | 0.803 | 0.830 | 3.219 | 3.406 |
| 1:32k | 0.105 | 0.108 | 0.496 | 0.533 | 2.456 | 2.510 |
| 1:64K | 0.092 | 0.101 | 0.297 | 0.290 | 1.623 | 1.548 |
| PBS | 0.106 | 0.104 | 0.111 | 0.119 | 0.119 | 0.144 |

After the 4th and 5th immunization, peripheral blood mononuclear cells (PBMCs) were obtained from the two immunized alpacas, respectively. Then, RNA was extracted from the PBMCs, and RNA was reverse transcribed into cDNA. VHH sequences were then amplified from cDNA using the single domain antibody cloning primer combinations and subcloned into the yeast display vector pYDisplay, which were electro-transformed to EYB100 competent cells to construct single domain antibody yeast display libraries.

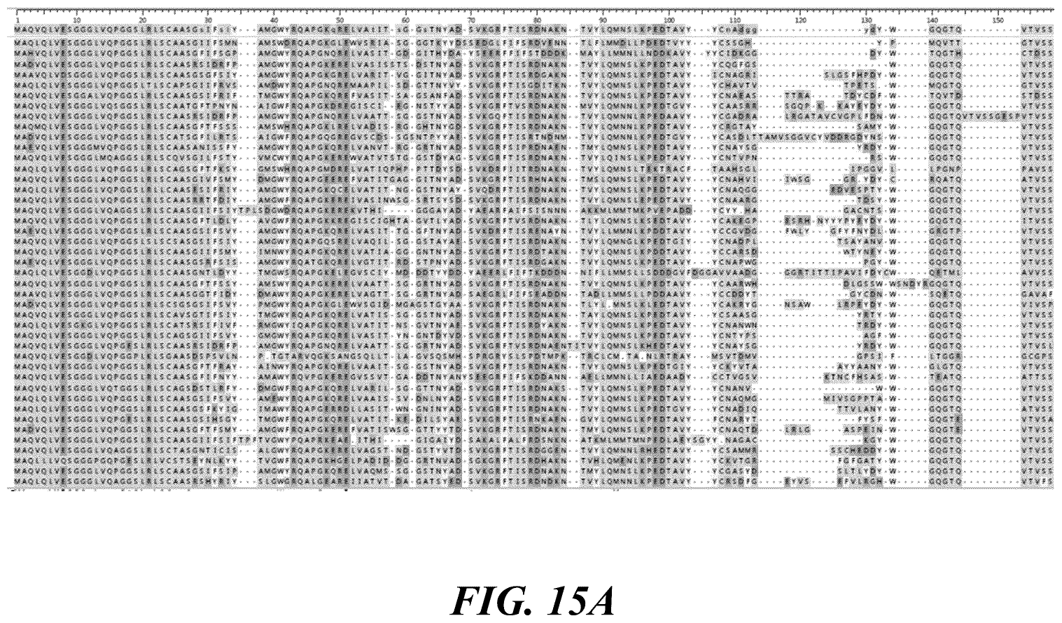

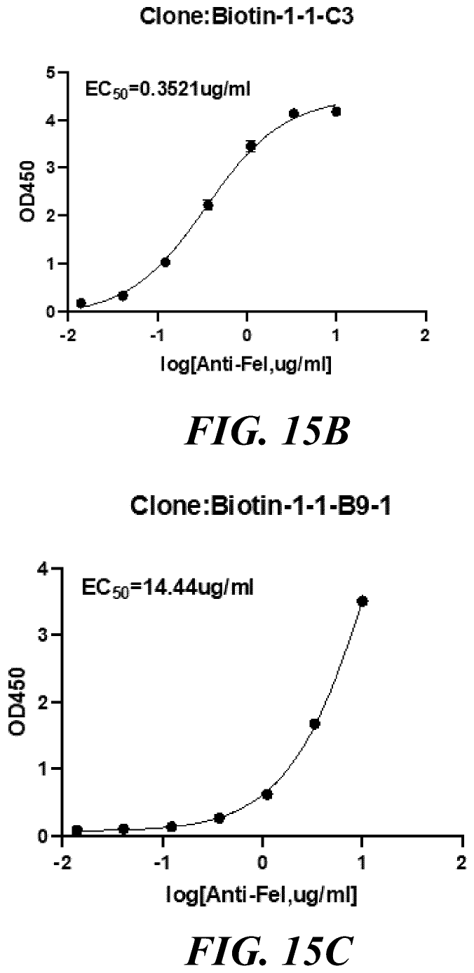

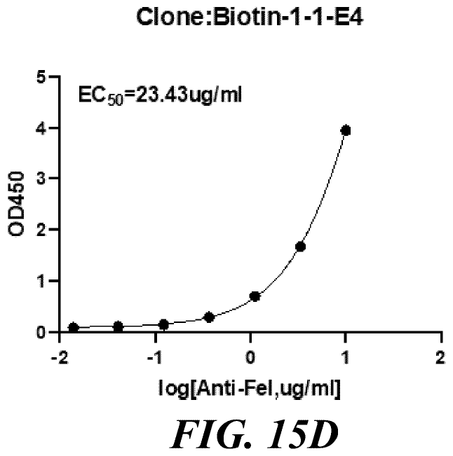

The sequences of 50 randomly selected clones demonstrated low overlap, indicating a diverse library, as shown in FIG. 15A. Single clones were subjected to a flow cytometry-based assay in which biotinylated recombinant Fel D1 was incubated with the VHH clone and following a wash step, detection of bound Fel D1 identified the clone as a VHH candidate. VHH candidates were then constructed into prokaryotic expression vectors.

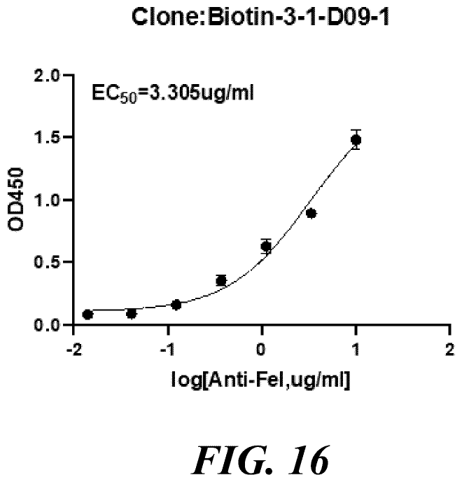

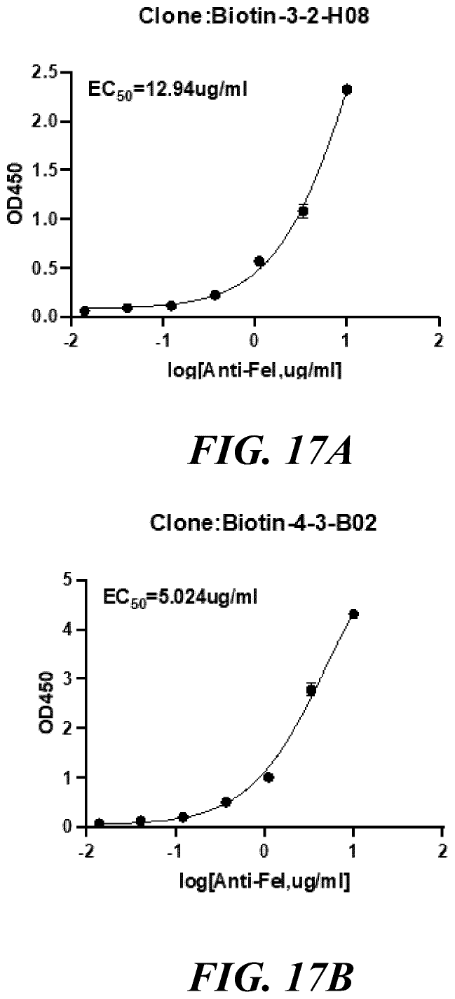

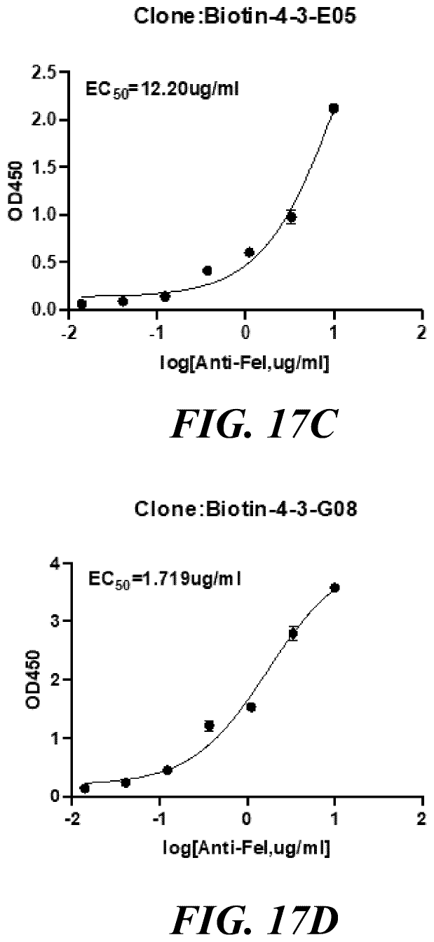

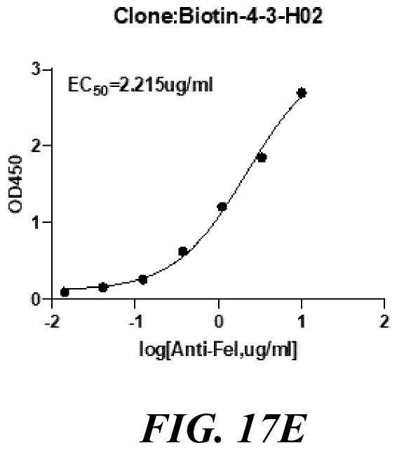

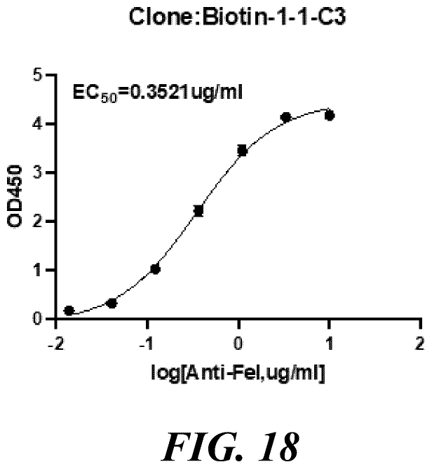

Isolated VHH clones were further validated by performing an ELISA assay as described in Example 1. Clones were incubated with each of the epitopes listed in Table 20. Binding results are shown in FIG. 15B-15D for the epitope of SEQ ID NO: 61. Binding results are shown in FIG. 16 for the epitope of SEQ ID NO: 59. Binding results are shown in FIG. 17A-17E for the epitope of SEQ ID NO: 60. Binding results are shown in FIG. 18 for the epitope of SEQ ID NO: 58. Sequence information for the VHH clones and the binding assay results are summarized in Table 22. Each clone demonstrated strong binding, indicated by low EC50 values.

TABLE 22
Anti-Fel d1 VHH Validation

| | | | | EC₅₀ | | |
|---|---|---|---|---|---|---|
| | SEQ ID | | Epitope | (μg/ | | |
| Clone | NO: | Sequence | Recognized | mL) | T_{agg} | Tₘ |
| SDAB22 | 49 | QLQLVESGGGLVQPGGSLRLSC | ENARILK | 0.352 | 58.93 | 66.50 |
| 110-1-1- | | EASGFTFKYYTMSWYRQAPGK | NCVDAK | | | |
| C3 | | ERELVATITNGDRTNYADSVK | M (SEQ ID | | | |
| | | GRFTISRDNAKNTLYLQMNSL | NO: 61), | | | |
| | | KPYDTAVYYCNRHLPLLQIWG | VAQYKA | | | |
| | | QGTQVTVSS | LPVVLEN | | | |
| | | | A (SEQ ID | | | |
| | | | NO: 58) | | | |
| SDAB22 | 50 | QVQLVESGGGLVQPGGSLRLS | ENARILK | 14.440 | 41.73 | Tm1: |
| 110-1-1- | | CAASGFTFRLAAMGWYRQAPE | NCVDAK | | | 54.03 |
| B9-1 | | KEREWVASITGPGTDTNYADS | M (SEQ ID | | | Tm2: |
| | | VKGRFTVSRDNAKNTVYLQM | NO: 61) | | | 63.20 |
| | | NSLKPEDTAVYYCRGMGYWG | | | | Possible |
| | | KGTLVTVSS | | | | aggregates |
| SDAB22 | 51 | AVQLVDSGGGLVQAGGSLRLA | ENARILK | 3.305 | 42.78 | Tm1: |
| 110-1-1- | | CAASGRTFDTYAVGWFRQAPG | NCVDAK | | | 41.13 |
| E4 | | KERDVVASITWTSGSTWYADF | M (SEQ ID | | | Tm2: |
| | | VKGRFTISKDNAKNTVYLQMN | NO: 61) | | | 52.88 |
| | | NLSPEDTAVYYCGARNQIYTR | | | | Possible |
| | | WDSWGQGTQVTVSS | | | | aggregates |
| SDAB22 | 52 | QVKLEESGGGLVQAGGSLRLS | FAVANG | 12.940 | 42.30 | 47.50 |
| 110-3-1- | | CVASGRTFSRWHTGWYRQAP | NELLLDL | | | |
| D9-1 | | GREREFVATLRASGGDTYYAD | S (SEQ ID | | | |
| | | SVKGRFTISRDNAKNTVYLQM | NO: 59) | | | |
| | | NSLKPEDTAVYYCNVWANWG | | | | |
| | | APPSDFSSWGQGTQVTVSS | | | | |
| SDAB22 | 53 | QLQLVESGGGLVQPGGSLRLSC | AKMTEE | 5.024 | 35.68 | 42.43 |
| 110-3-2- | | AASGFTFSSYSMSWYRQAPGK | DKENALS | | | |
| H08 | | ERELVATIDTDGRTNYADSVK | (SEQ ID | | | |
| | | GRFTISRDNAKNSVYLQMNSL | NO: 60) | | | |
| | | KPEDTAVYYCNRRQLGVDYW | | | | |
| | | GQGTQVTVSS | | | | |
| SDAB22 | 54 | QVQLVESGGGLVQPGGSLRLS | AKMTEE | 12.200 | 40.93 | 46.83 |
| 110-4-3- | | CAASGFTFSNYGMSWVRQAPG | DKENALS | | | |
| B02 | | KGLEWVSDVNSSGSRRFYVDS | (SEQ ID | | | |
| | | VKGRFTISRDNAKNTVFLQMN | NO: 60) | | | |
| | | SLKPEDSAVYYCVKLAEAGTLI | | | | |
| | | HVGSWGQGTRVTVSS | | | | |
| SDAB22 | 55 | QLQLVESGGGLVQPGGSLRLSC | AKMTEE | 1.719 | 31.74 | Tm1: |
| 110-4-3- | | VGSGFTFSLASMGWYRQAPGK | DKENALS | | | 37.85 |
| E05 | | EREWVASISSLDASTNYADSVK | (SEQ ID | | | Tm2: |
| | | GRFTISRDNAKRMVYLQMNNL | NO: 60) | | | 47.65 |
| | | KSEDTAVYYCKAMNYWGKGT | | | | Tm3: |
| | | QVTVSS | | | | 62.39 |
| | | | | | | Possible |
| | | | | | | aggregates |
| SDAB22 | 56 | QLQLVESGGGLVQPGGSLRLSC | AKMTEE | 2.215 | 42.29 | 58.58 |
| 110-4-3- | | AASRSIFSSYVMAWYRRAPGK | DKENALS | | | |
| G08 | | KRELVASIANVGSNTDYASFAK | (SEQ ID | | | |
| | | GRFTISRDDDKSRDNDKITVYL | NO: 60) | | | |
| | | QMNSLNPEDTAVYYCNAWLG | | | | |
| | | AGSDYWGQGTQVTVSS | | | | |
| SDAB22 | 57 | QLQLVESGGGLVQPGGSLRLSC | AKMTEE | 12.940 | 42.30 | 47.50 |
| 110-4-3- | | EASGFTFKYYTMSWYRQAPGK | DKENALS | | | |
| H02 | | ERELVATITNGDRINYADSVK | (SEQ ID | | | |
| | | GRFTISRDNAKNTLYLQMNSL | NO: 60) | | | |
| | | KPYDTAVYYCNRHLPLLQIWG | | | | |
| | | QGTQVTVSS | | | | |

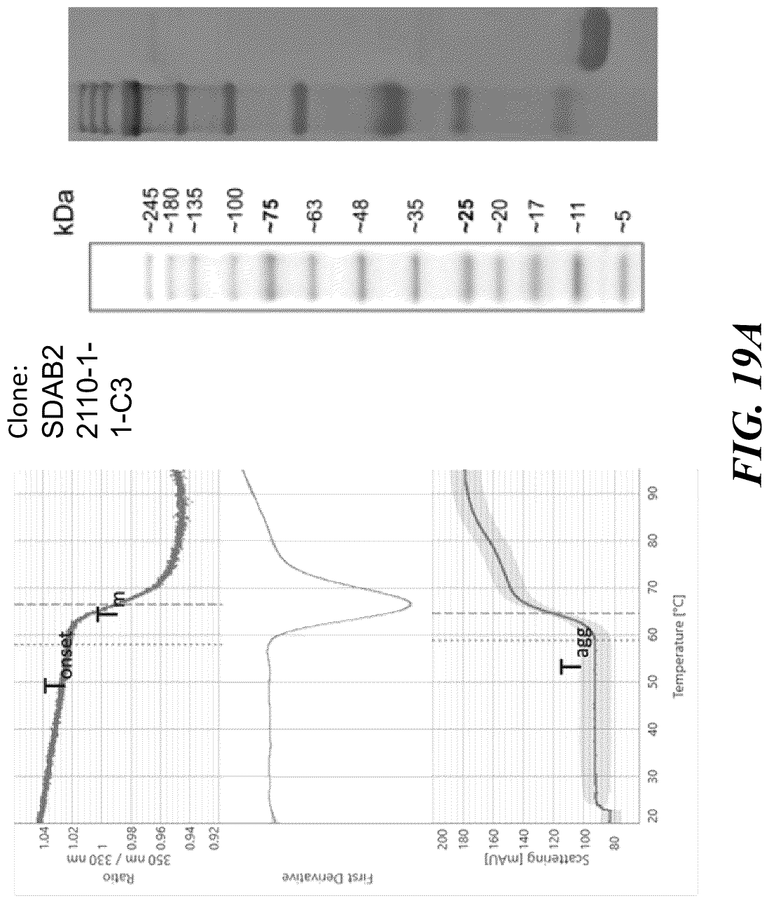

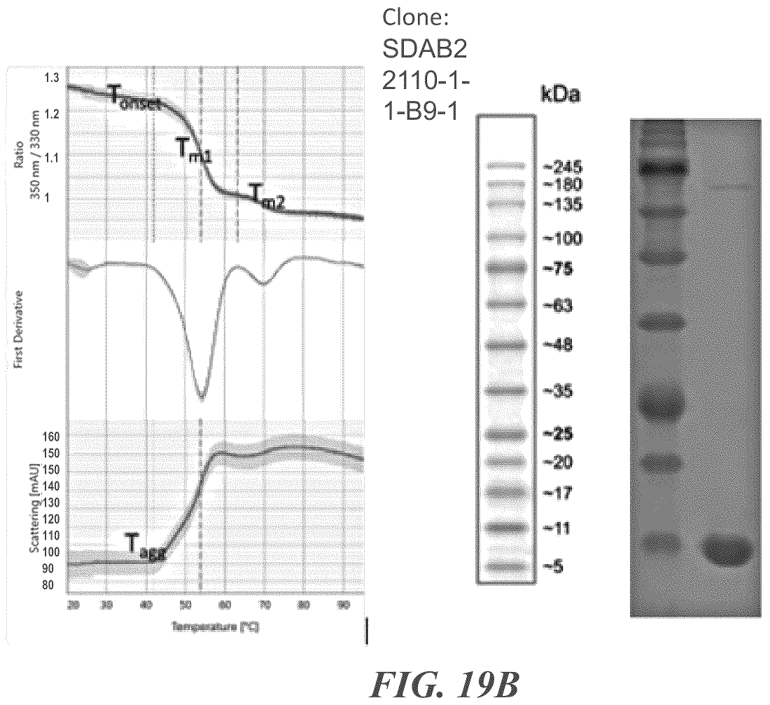

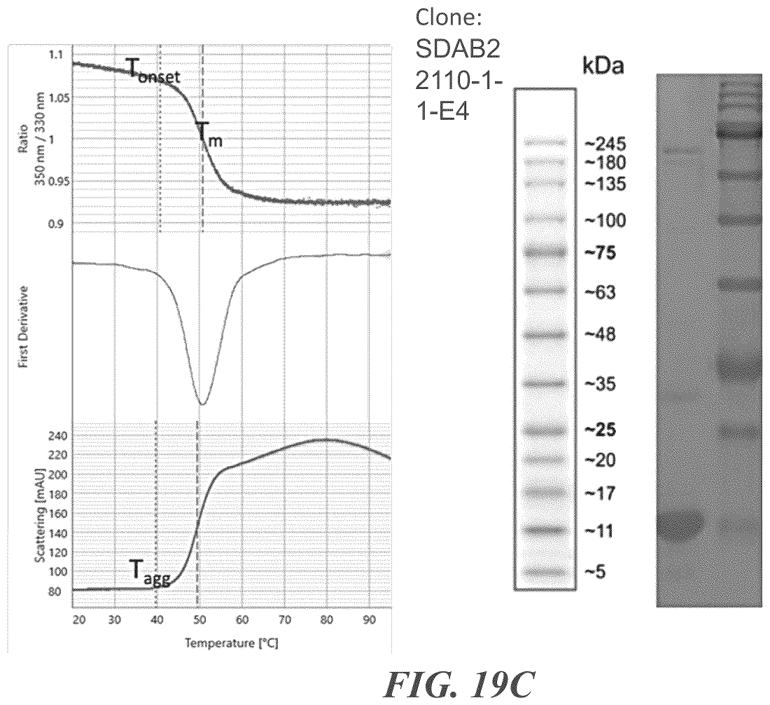

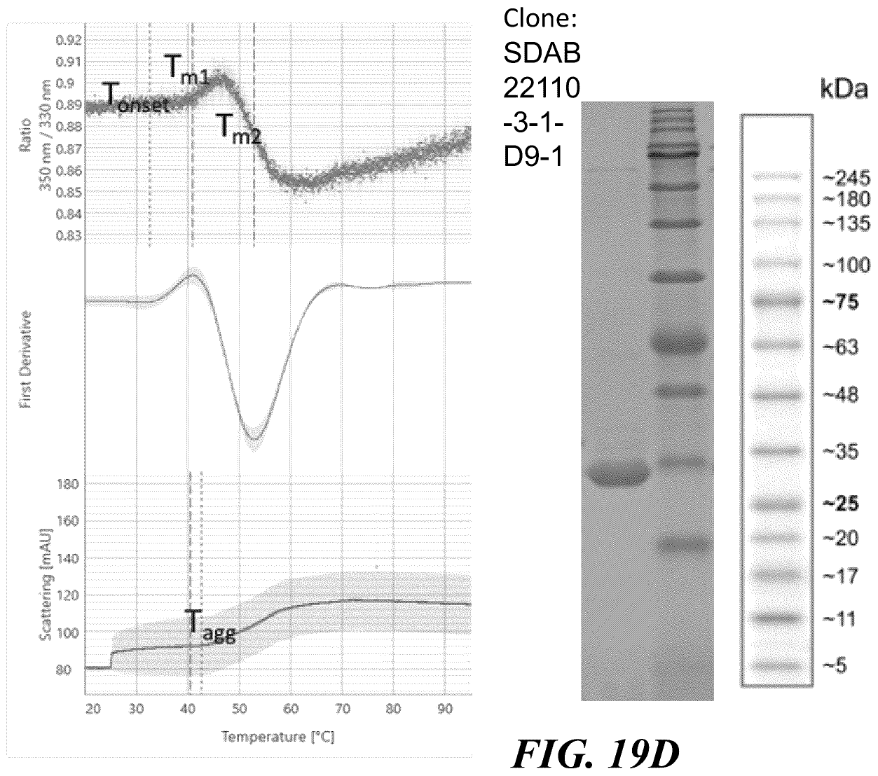

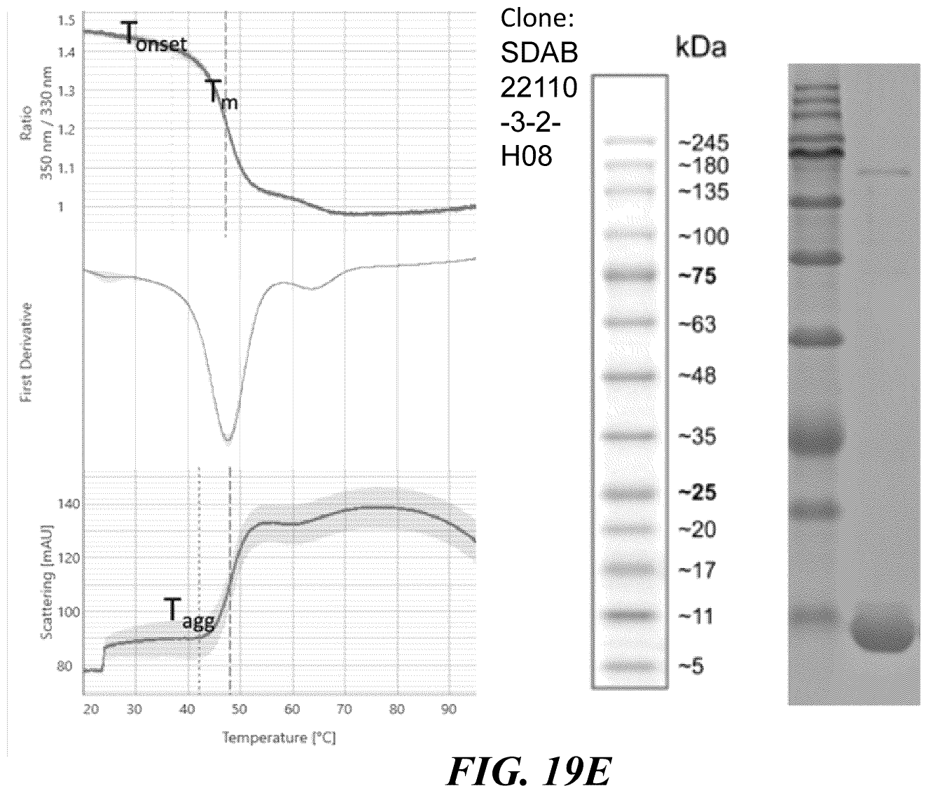

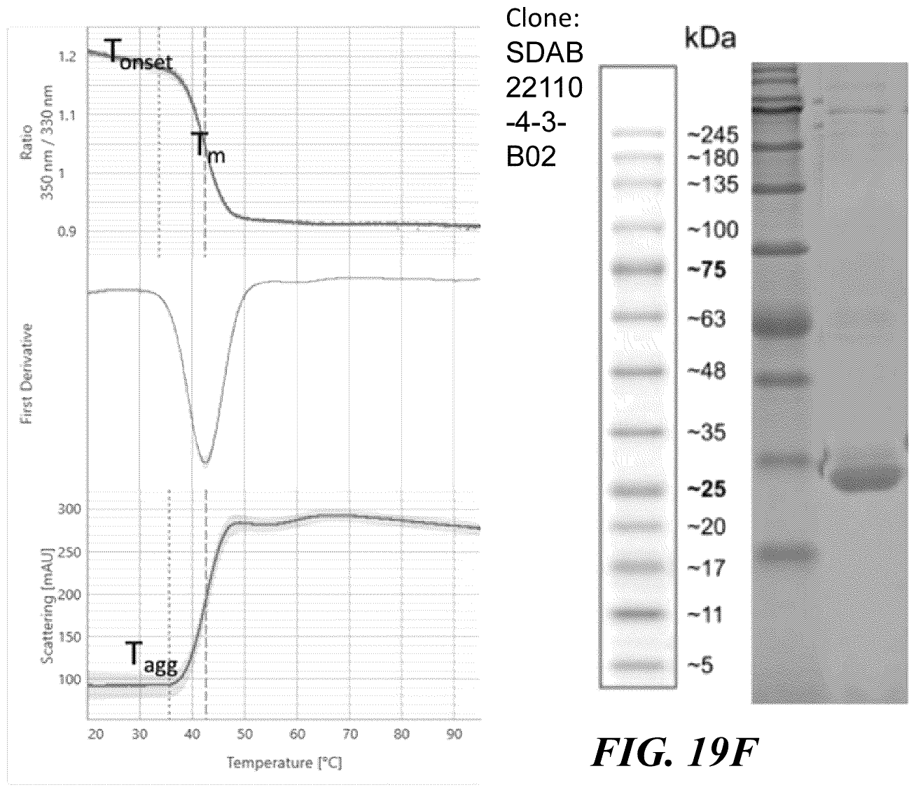

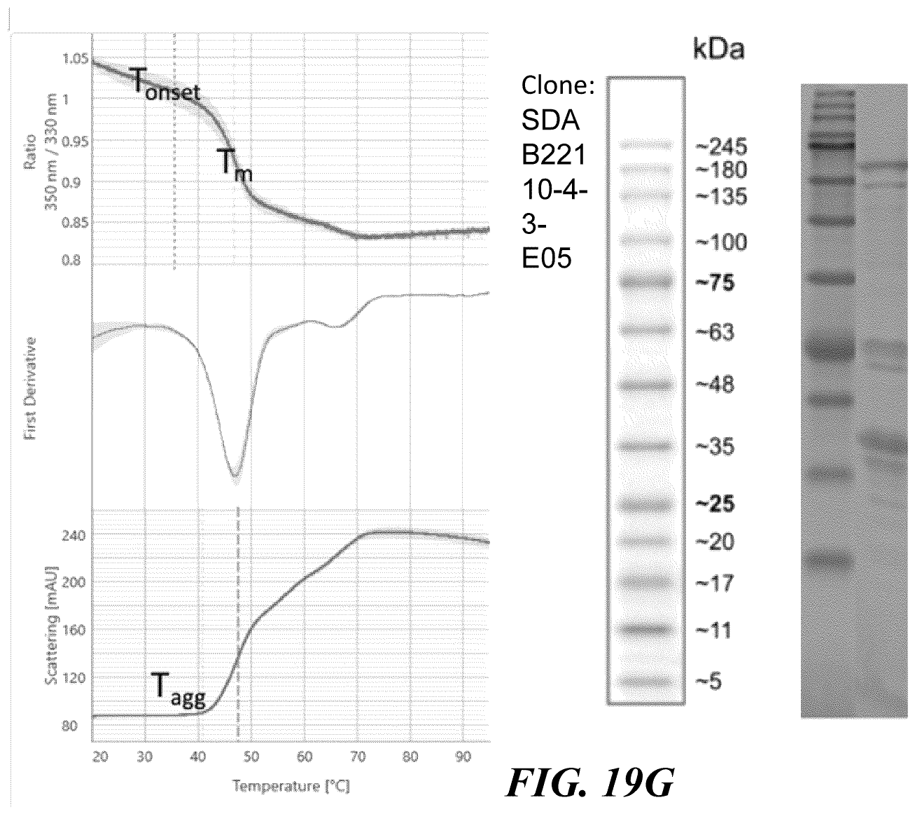

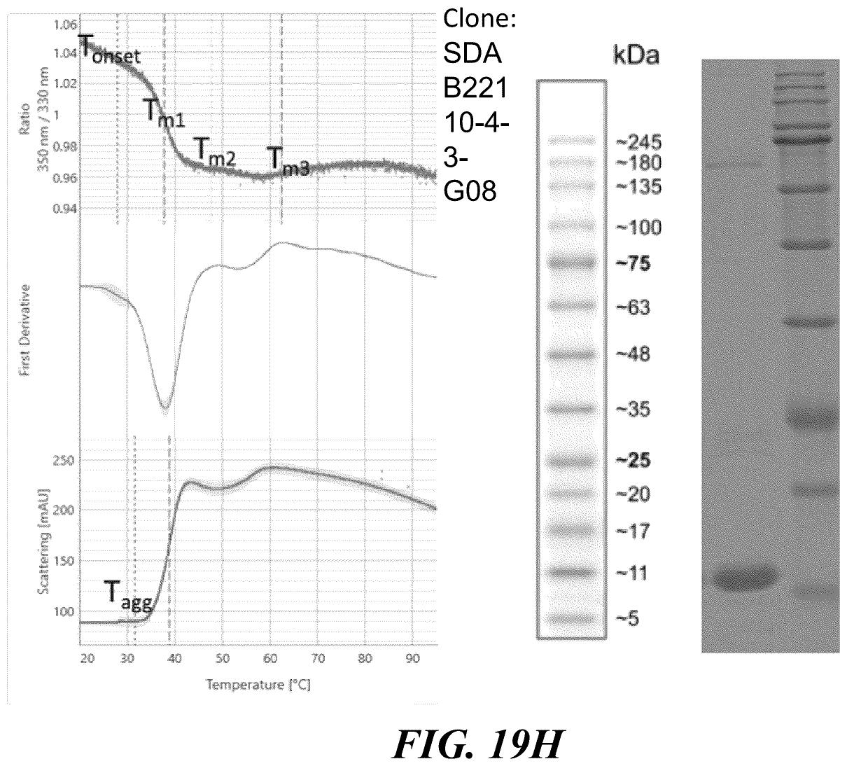

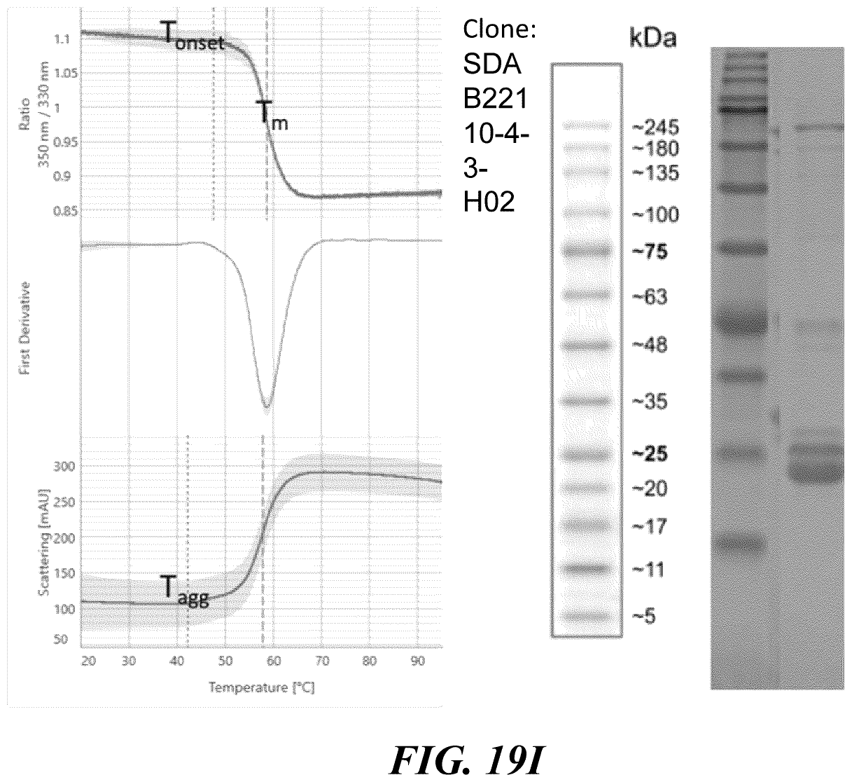

The detailed thermostability graphs used to determine the Tm, Tonset, and Tagg are illustrated in FIGS. 19A-19I and raw data is summarized in Table 23. Any subsequent melting points observed (Tₘ1, Tₘ2, Tₘ3, etc.) are separated by commas.

TABLE 23
Thermostability Results for Anti-Fel d1 VHH

| Clone: | Tₘ | | | | | |
|---|---|---|---|---|---|---|
| DAB22110- | ø | σ | Tₒₙₛₑₜ | | T_{agg} | |
| 1-1-C3 | (mean) | (std. dev) | ø | σ | ø | σ |
| Average | 66.50 | 0.01 | 58.06 | 0.16 | 58.93 | 0.12 |
| Replicate 1 | 66.50 | | 58.17 | | 59.02 | |
| Replicate 2 | 66.51 | | 57.94 | | 58.85 | |

| | Tₘ | | Tₒₙₛₑₜ | | T_{agg} | |
|---|---|---|---|---|---|---|
| | ø | σ | ø | σ | ø | σ |
| Clone: | | | | | | |
| SDAB22110- | | | | | | |
| 1-1-B9-1 | | | | | | |
| Average | 54.03, 63.20 | 0.11, 0.08 | 42.07 | 0.05 | 41.73 | 0.00 |
| Replicate 1 | 53.95, 63.26 | | 42.11 | | 41.73 | |
| Replicate 2 | 54.10, 63.15 | | 42.04 | | 41.73 | |
| Clone: | | | | | | |
| SDAB22110- | | | | | | |
| 1-1-E4 | | | | | | |
| Average | 50.69 | 0.05 | 40.64 | 0.07 | 39.61 | 0.00 |
| Replicate 1 | 50.73 | | 40.60 | | 39.62 | |
| Replicate 2 | 50.66 | | 40.64 | 0.07 | 39.61 | |
| Clone: | | | | | | |
| SDAB22110- | | | | | | |
| 3-1-D9-1 | | | | | | |
| Average | 41.13, 52.88 | 0.17, 0.03 | 32.74 | 0.23 | 42.78 | 0.03 |
| Replicate 1 | 41.01, 52.86 | | 32.57 | | 42.76 | |
| Replicate 2 | 41.25, 52.91 | | 32.90 | | 42.80 | |
| Clone: | | | | | | |
| SDAB22110- | | | | | | |
| 3-2-H08 | | | | | | |
| Average | 47.50 | 0.05 | 37.18 | 0.22 | 42.30 | 0.16 |
| Replicate 1 | 47.46 | | 37.03 | | 42.41 | |
| Replicate 2 | 47.53 | | 37.34 | | 42.19 | |
| Clone: | | | | | | |
| SDAB22110- | | | | | | |
| 4-3-B02 | | | | | | |
| Average | 42.43 | 0.12 | 33.78 | 0.00 | 35.68 | 0.19 |
| Replicate 1 | 42.52 | | 33.78 | | 35.55 | |
| Replicate 2 | 42.34 | | 33.78 | | 35.82 | |
| Clone: | | | | | | |
| SDAB22110- | | | | | | |
| 4-3-E05 | | | | | | |
| Average | 46.83 | 0.27 | 35.67 | 0.08 | 40.93 | 0.00 |
| Replicate 1 | 47.02 | | 35.61 | | 40.93 | |
| Replicate 2 | 46.64 | | 35.73 | | 40.93 | |
| Clone: | | | | | | |
| SDAB22110- | | | | | | |
| 4-3-G08 | | | | | | |
| Average | 37.85, | 0.10, | 27.92 | 0.00 | 31.74 | 0.04 |
| | 47.65, 62.92 | 0.16, 0.75 | | | | |
| Replicate 1 | 37.77, | | 27.93 | | 31.71 | |
| | 47.76, 61.85 | | | | | |
| Replicate 2 | 37.92, | | 27.92 | | 31.77 | |
| | 47.53, 62.92 | | | | | |
| Clone: | | | | | | |
| SDAB22110- | | | | | | |
| 4-3-H02 | | | | | | |
| Average | 58.58 | 0.02 | 47.54 | 0.31 | 42.29 | 0.08 |
| Replicate 1 | 58.60 | | 47.33 | | 42.24 | |
| Replicate 2 | 58.57 | | 47.76 | | 42.35 | |

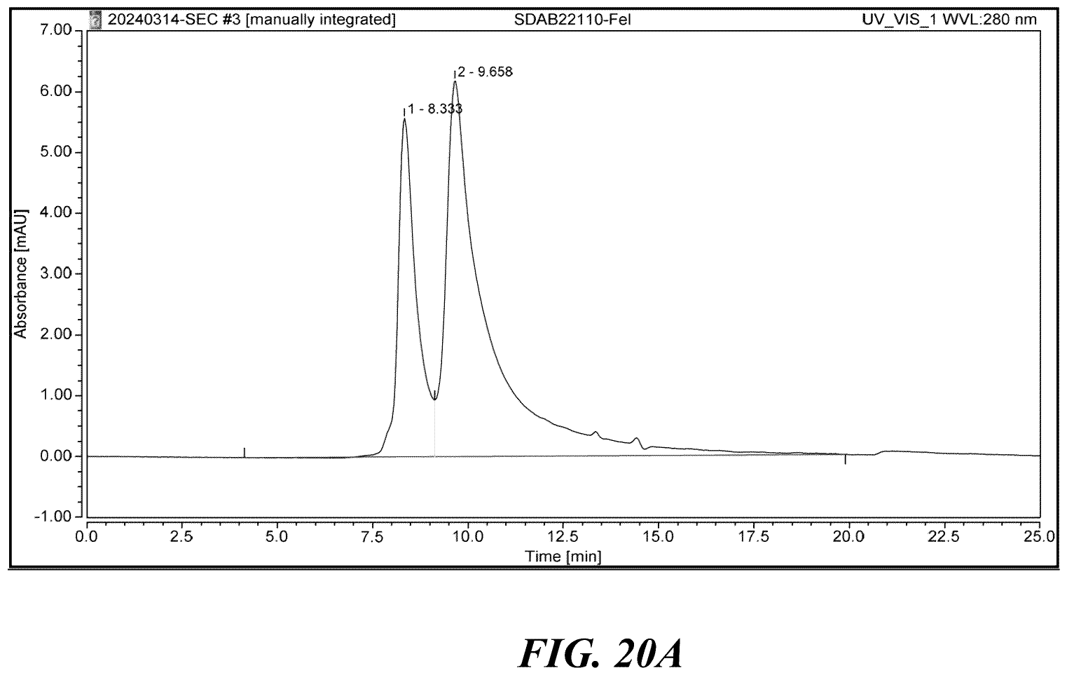

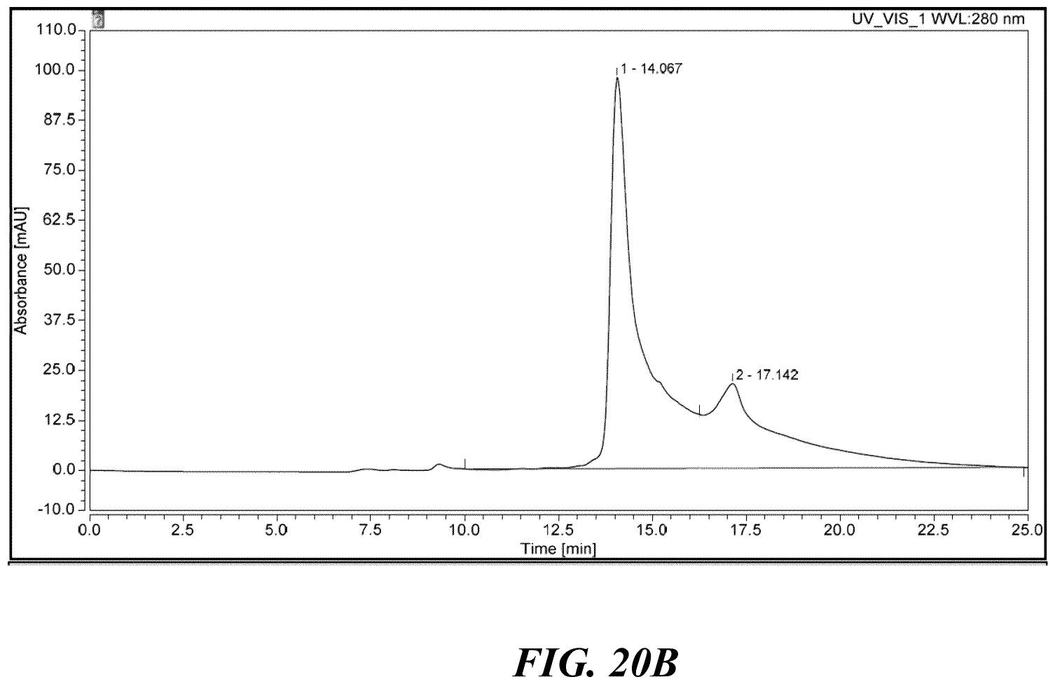

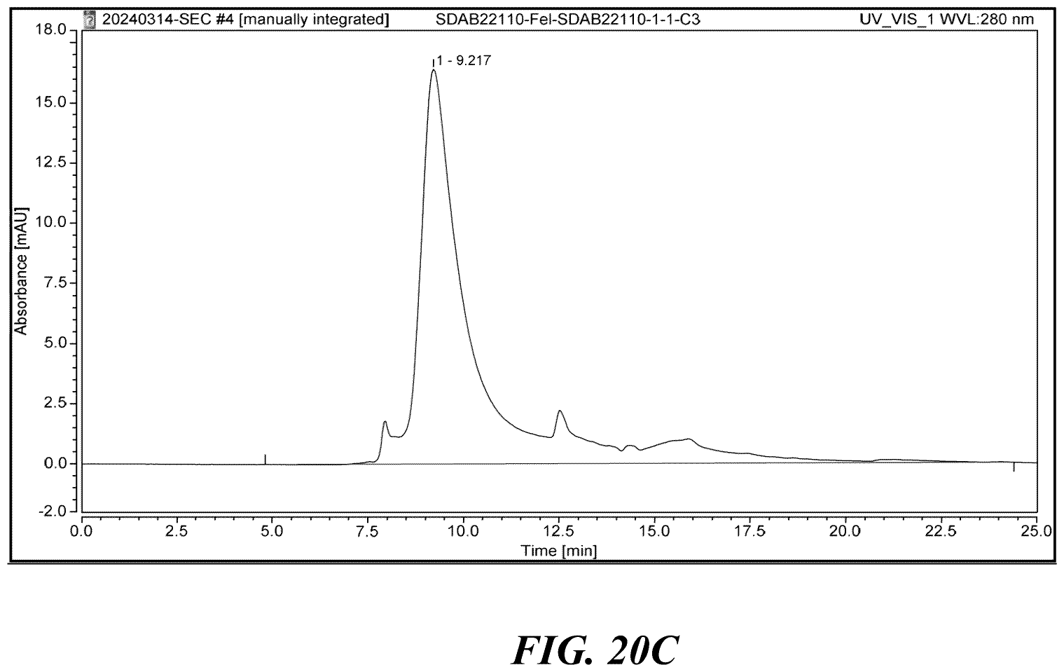

Functional validation was also performed testing the capability of an exemplary clone to neutralize Fel D1. Fel D1 is naturally a weaker tetramer that causes allergic reactions by causing cross-linking of neighboring IgEs on immune cells (mast cells, etc.). Size Exclusion Chromatography (SEC) was performed wherein proteins were run through a mesh where larger proteins do not flow into the mesh as readily and therefore elute earlier. On the SEC, there are two distinct peaks for pure Fel D1, the earlier peak representing the tetramer and the laker peak representing the monomer (FIG. 20A). Conditions for this SEC graph are (Column: Zenix-C SEC-80, 7.8×300 mm, 3 um, 80 Å; Sample: Fel D1; Sample Volume: 30 μL (25 ug); Mobile phase: 150 mM phosphate buffer, pH 7.0; Flow rate: 0.7 mL/min; Detection: UV 280 nm; System: Thermo Vanquish Flex.

When clone C3 was assayed, it was observed to be smaller than the Fel D1 monomer, having a main peak around 14 minutes (FIG. 20B). Conditions for this SEC graph are (Column: Zenix-C SEC-80, 7.8×300 mm, 3 um, 80 Å; Sample: 1-C3; Sample Volume: 30 μL; Mobile phase: 150 mM phosphate buffer, pH 7.0; Flow rate: 0.7 mL/min; Detection: UV 280 nm; System: Thermo Vanquish Flex.

When clone C3 and Fel D1 are incubated together for 4 hours at 37° C. and then assayed, the two Fel D1 peaks were no longer detected and only the C3-Fel D1 complex is detected. Notably, this complex appears at a later time than the Fel D1 tetramer, demonstrating that the C3 nanobody is preventing the Fel D1 tetramer from forming (FIG. 20C). Conditions for this SEC graph are (Column: Zenix-C SEC-80, 7.8×300 mm, 3 um, 80 Å; Sample: 30 ug Fel D1 protein and 30 ug 1-C3 were pre-incubated at 37° C. for 4 hours in a 60 ul incubation system; Sample Volume: 30 μL (25 ug); Mobile phase: 150 mM phosphate buffer, pH 7.0; Flow rate: 0.7 mL/min; Detection: UV 280 nm; System: Thermo Vanquish Flex.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-196. (canceled)

197. An allergen binding protein that binds to or neutralizes an allergen, wherein the allergen binding protein comprises a nanobody, a monobody, a DARPin, or a small peptide below about 25 kilodalton in molecular weight or under about 300 amino acids in length, wherein the nanobody or the small peptide has at least 80% sequence identity to a sequence selected from the group consisting of SEQ ID NOs: 1-57 and 67-73.

198. The allergen binding protein of claim 197, wherein the nanobody or the small peptide has at least 85%, at least 90%, at least 95%, at least 97%, at least 99%, or 100% sequence identity to a sequence selected from the group consisting of SEQ ID NOs: 1-57 and 67-73.

199. The allergen binding protein of claim 197, wherein the allergen binding protein is formulated for coating an animal food or spraying, misting, or brushing an animal or household surface, wherein the allergen binding protein is at a concentration of about 0.01 milligram per milliliter (mg/ml) to about 500 mg/ml in solution or after suspension.

200. The allergen binding protein of claim 197, wherein the allergen comprises an environmental allergen or an animal allergen.

201. The allergen binding protein of claim 200, wherein the animal allergen is selected from the group consisting of Fel d 1, Fel d 2, Fel d 3, Fel d 4, Can f 1, Can f 2, Can f 4, Can f 7, Ory C 1, Mus M 1, and Bla G 2.

202. The allergen binding protein of claim 201, wherein the animal allergen is selected from the group consisting of Fel d 1, Can f 1, and Can f 2.

203. The allergen binding protein of claim 202, wherein the allergen binding protein binds to or neutralizes Fel d 1 and comprises the nanobody comprising an amino acid sequence having at least 80%, at least 85%, at least 90%, at least 95%, at least 97%, at least 99%, or 100% sequence identity to a sequence selected from the group consisting of SEQ ID NOs: 49-57.

204. The allergen binding protein of claim 202, wherein the allergen binding protein binds to or neutralizes Can f 1 and comprises the nanobody comprising an amino acid sequence having at least 80%, at least 85%, at least 90%, at least 95%, at least 97%, at least 99%, or 100% sequence identity to a sequence selected from the group consisting of SEQ ID NOs: 67-73.

205. The allergen binding protein of claim 202, wherein the allergen binding protein binds to or neutralizes Can f 2 and comprises the nanobody comprising an amino acid sequence having at least 80%, at least 85%, at least 90%, at least 95%, at least 97%, at least 99%, or 100% sequence identity to a sequence selected from the group consisting of SEQ ID NOs: 9-23.

206. The allergen binding protein of claim 200, wherein the environmental allergen comprises a dust allergen comprising Der p1 or Der p2.

207. The allergen binding protein of claim 206, wherein the allergen binding protein binds to or neutralizes Der p1 and comprises the nanobody comprising an amino acid sequence having at least 80%, at least 85%, at least 90%, at least 95%, at least 97%, at least 99%, or 100% sequence identity to a sequence selected from the group consisting of SEQ ID NOs: 24-34, or the small peptide having least 80%, at least 85%, at least 90%, at least 95%, at least 97%, at least 99%, or 100% sequence identity to a sequence selected from the group consisting of SEQ ID NOs: 35-48.

208. The allergen binding protein of claim 206, wherein the allergen binding protein binds to or neutralizes Der p2 and comprises the nanobody having at least 80%, at least 85%, at least 90%, or 100% sequence identity to a sequence selected from the group consisting of SEQ ID NOs: 1-8.

209. The allergen binding protein of claim 197, wherein the allergen comprises a plant or plant pollen allergen, wherein the plant or pollen allergen is selected from the group consisting of Bet v1, Phl p 5, Phl p 1, Poa p 1, Cyn d 1, Bet v 2, Ole e 1, Amb a 1, Amb a 11, and Art v 1.

210. The allergen binding protein of claim 197, wherein the allergen comprises a mold allergen, wherein the mold allergen is selected from the group consisting of Alt a 1, Asp f 1, Asp f 2, Cla h 8, Pen ch 13, and Pen ch 18.

211. The allergen binding protein of claim 197, wherein the allergen is a food allergen, wherein the food allergen is selected from the group consisting of Pen a 1, Ara h 1, Ara h 3.

212. The allergen binding protein of claim 197, wherein the allergen binding protein comprises at least one modified amino acid.

213. The allergen binding protein of claim 212, wherein the allergen binding protein comprises about 1, about 5, about 10, about 15, about 20, about 25 or about 30 modified amino acids, wherein the modified amino acid comprises a non-canonical amino acid.

214. The allergen binding protein of claim 213, wherein the non-canonical amino acid is selected from the group consisting of para-benzoylphenylalanine, 3,4-dyhydroxyphenylalanine, a tetrazine, a clooctene, homopropargylglycine, para-proparglyoxyphenylalanine, para-azidophenylalanine, para-isothiocynate phenylalanine, para-benzoylphenylalanine, para-cyanophenylalanine, para-nitrophenylalanine, a m-halogenated tyrosine analog, a halogenated proline analog, a halogenated tryptophan analog, and a halogenated leucine analog.

215. The allergen binding protein of claim 212, wherein the allergen binding protein provides (i) solubility increased by about 25%, 50%, 75%, 100%, 200%, 300%, 400%, 500%, 600% or more compared to an unmodified allergen binding protein, (ii) binding affinity increased by about 25%, 50%, 75%, 100%, 200%, 300%, 400%, 500%, 600%, 700%, 800%, 900%, 1000%, 1500%, or more compared to an unmodified allergen binding protein, or (iii) thermal stability increased by about 10%, 25%, 50%, 75%, 100%, 200%, 300%, or more compared to an unmodified allergen binding protein.

216. The allergen binding protein of claim 197, wherein the allergen binding protein is a multivalent allergen binding protein, wherein binding affinity of the multivalent allergen binding protein is increased by about 25%, 50%, 75%, 100%, 200%, 300%, 400%, 500%, 600%, 700%, 800%, 900%, 1000%, 1500%, or more compared to a monovalent allergen binding protein.


SEQUENCE LISTING (73 sequences)

SEQ ID NO: 1 - protein, 115 aa, synthetic construct
QLQLVESGGGLVQPGGSLRLSCVVSGRTLSGHNMGWFRQAPGKEREFVARINSNGGTTRYADAVKGRFTISRDNAKNTAYLQMNSLKSEDTAVYYCRLVGPAGYWGQGTQVTVSS

SEQ ID NO: 2 - protein, 121 aa, synthetic construct
DVQLVESGGGLVQPGGSLRLSCVASGLTFSYYDMKWHRQAPGKERELVAAIIEGGRTMYADSVKGRFTISRDSPKKSVYLQMNNLKSEDTAVYYCNAVRARFRGLGNDDAWGQGTQVTVSS

SEQ ID NO: 3 - protein, 115 aa, synthetic construct
QVQLVESGGGLVQPGGSLRLSCAASASFVGAYAMYWFRQATGKQRELVAFITSGNSTNYADSVKGRFTISRDYTKNTVYLQMNKLKPEDTAVYSCNLVTSRGDYWGQGTQVTVSS

SEQ ID NO: 4 - protein, 119 aa, synthetic construct
EVQLVESGGGLVQPGGSLRLSCAASGRTFSSYAMGWFRQAPGKEREFVAGISRRGSNIYYADSVKGRFTISRDNAKNTVYLQMNSLKPEDTAVYYCNARHPRISTRLYWGQGTQVTVSS

SEQ ID NO: 5 - protein, 117 aa, synthetic construct
QVQLVESGGGLVQPGGSLRLSCAASRSIDRFPAMGWYRQAPGKKRELVAAISSANTRTKYADSVKGRFTISRDNAENTMYLQMNSLKPEDTAVYYCNVFPRPVQGYWGQGTQVTVSS

SEQ ID NO: 6 - protein, 122 aa, synthetic construct
QVQLVESGGGLVQPGGSLRLSCAASGRIFRPDGMAWYRQAPGKQREFVAGITRGGRTTYADSVKGRFTISRDNAENTVYLQMNSLKPEDTAVYYCNAKPSAWTLPRYRYDYWGQGTQVTVSS

SEQ ID NO: 7 - protein, 118 aa, synthetic construct
DVQLVESGGGLVQPGGSLRLSCAASGFTFALYSMRWYRQAPGKERELVAAITSGRSTNYADSVKGRFTISRDNAKNTVYLQMNSLKPEDTAVYHCNARHPIATLGNYWGQGTPVTVSS

SEQ ID NO: 8 - protein, 115 aa, synthetic construct
QLQLVESGGGMVQPGGSLRLSCAASGTLFSTALMGWYRQAPGKQRTLVASITDGGSTNYVDSVKGRFTISRDNEKNTVYLQMNSLKFEDTAVYYCNAHWGSYDYWGQGTQVVVSS

SEQ ID NO: 9 - protein, 122 aa, synthetic construct
QVQLVESGGGLVQPGGSLRLSCAYSGFTLDNNVIGWFRQAPGKEREFVAAISRSGAFTHYTESVQGRFTISRDNAKNTVYLQMNSLKPEDTAVYYCAAGAILLPTERRYDYWGQGTQVTVSS

SEQ ID NO: 10 - protein, 116 aa, synthetic construct
AVQLVESGGGLVQPGGSLRLSCAASGFTFSNYYMSWYRQAPGKEREWVAFITSTGSSTNYANSVKGRFTASRDNAKNTVYLQMNSLKPEDTAVYYCKTRLWGNDYWGKGTLVTVSS

SEQ ID NO: 11 - protein, 122 aa, synthetic construct
QVQLVESGGGLVQAGGSLGLSCAASGRTFNNYVMGWFRQAPGKEREFVAAISRSGSFTHYAEAVQGRFTISRDNAKITVYLQMNSLKPEDTAVYYCAAGAILMPTERTYDYWGQGTQVTVSS

SEQ ID NO: 12 - protein, 114 aa, synthetic construct
EVQLVESGGGLVQPGGSLRLSCVVSGSIFSDNAMGWYRQAPGKQREMVAIISSVGTTNNVDSVNGRFTISRDNAKNTVYLQMNSLKPEDTAVYYCKDFSAPRYWGQGTQVTVSS

SEQ ID NO: 13 - protein, 121 aa, synthetic construct
AVQLVDSGGGLVQAGDSLRLSCVASGRTFSSYVMGWFRQAPGKERLIVATISKSGSLTHYADSVEGRFTISRDNAKNTVYLQMNSLEPEDTAVYYCTPVSDLTGRRLGSYWGQGTQVTVSS

SEQ ID NO: 14 - protein, 124 aa, synthetic construct
AVQLVDSGGGLVQPGGSLRLSCAASGRTFSYYAMGWFRQALGKEREFVAASSRTGRVTNYADSVKGRFTISRDNAKNTVYLQMNSLKPEDTAIYYCAADDRFYGGDNPAFYNSWGQGTQVTVSS

SEQ ID NO: 15 - protein, 124 aa, synthetic construct
QVQLVESGGGLVQAGDSLRLSCAASGRSFSNYVMNWFRQAPGKEREFVAAISRSGRSTFYADSVKGRFTISRDNPKATVYLQMNSLVIEDTAVYFCAAASDITSMRSLAAITSWGQGTQVIVSS

SEQ ID NO: 16 - protein, 122 aa, synthetic construct
AVQLVESGGGLVQAGGSLRLSCAASGRTFNNSVMGWFRQAPGKEREFVAAISRSGAFAHYAESVEGRFTISRDNAKNTVYLQMNSLKPEDTAVYYCAAGAILLPTERRYDYWGQGTQVTVSS

SEQ ID NO: 17 - protein, 120 aa, synthetic construct
QVQLVESGGGLVQPGESLRLSCAASGSIFSIYRMGWYRQAPGEQREHVATVTSGGGTGYADSVKGRFTIYRDNAKNTVYLQMNSLKPEDTAVYSCYAIPKSWSRTSEYSWGQGTQVTVSS

SEQ ID NO: 18 - protein, 125 aa, synthetic construct
EVQLVESGGGLVQARGSLRLSCAASGRTFNNYAMGWFRQAPGKEREFVAAIASNTGTTYYAGSVKGRFAISRDNAKNTVDLHMNSLKPEDTAVYYCALGPLRLGWWYDSRPYDYWGQGTQVTVSS

SEQ ID NO: 19 - protein, 117 aa, synthetic construct
QVQLVESGGGGVQAGGSLRLSCVASQSHFGYNVMGWYRQAPGRQRELVATIISSGGTNYADSVKGRFTISRDNAKNTVHLSMDSLNVEDTAVYFCYAKGVWLGREYWGQGTQVTVSS

SEQ ID NO: 20 - protein, 122 aa, synthetic construct
EVQLVESGGGLVQAGGSLRLSCAASGRTFSEYIMGWFRQAPGKERVFVSTISKSGAITNYADSVQGRFTISRDNAKNTVYLQMNSLKPEDSAVYYCAAGPLLSPAGRQYDYWGQGTQVTVSS

SEQ ID NO: 21 - protein, 120 aa, synthetic construct
AVQLVESGGGLVQPGGSLRLSCVASGSSSRNYAMGWYRQAPGNEREFVAVITSRGYTHYANSVSGRFTISRDHAKDTAYLQMNSLKPEDTAVYYCNTQAGLLPFMTVYDWGTGTQVTVSS

SEQ ID NO: 22 - protein, 121 aa, synthetic construct
EVQLVESGGGLVQPGGSLRLSCAASGTIFLINRMGWYRQAPGKQRELVATSFTSGNSTMYADSVKGRFTISRDNAKKTVYLQMNSLKPEDTAVYYCKARISRRYGNWDDYWGQGTQVTVSS

SEQ ID NO: 23 - protein, 124 aa, synthetic construct
AVQLVDSGGGLVQTGGSLRLSCAASGTIFSINRMAWYRQAPGKQRELVASIFFSGGITHYADFVKGRFTISRDNAKNTMYLQMNSLKPEDTAVYYCKGVIAPNYRRTSNFQDYWGQGTQVTVSS

SEQ ID NO: 24 - protein, 132 aa, synthetic construct
QLQLVESGGGLVQSGGSLRLSCVAKGGTNHPYPIGWFRQAPGKEQEGVLCIASGGQTQDSAHNPNWADSVKGRFTISRDDATHTVYLEMNNLKADDTAVYFCAAPREIYIDSRCATYKYEYWGQGTQVTVSP

SEQ ID NO: 25 - protein, 120 aa, synthetic construct
QVQLVESGGGLVQAGGSLRLSCAASGRTFSSYAMGWFRQAPGKEREFVAIISNTGGLTDYAHSVKGRFTISRDNAKNTGYLQMNSLKPEDTALYYCAADFLGPKSWPSYWGQGTQVTVSS

SEQ ID NO: 26 - protein, 135 aa, synthetic construct
QLQLVESGGGLVQPGGSLRLSCVISGGTFHPYPIGWFREAPGKEREGVLCINSGGQSEISANSAHDPKYADSVKGRFTISRDNAANTVYLQMNNLEPGDTAVYYCAAPREIYVDPYCPTYAYEYWGQGTQVTVSA

SEQ ID NO: 27 - protein, 125 aa, synthetic construct
QVQLVESGGGLVQAGGSLRLSCAASGRTFSRYAMGWFRQAPGKEREFVAAITGSGSRTYYADSIQGRFTISRDNAKNTVYLQMNSLKPEDTAVYYCVQGWAEATMTSLGEDYDYWGQGTQVTVSS

SEQ ID NO: 28 - protein, 124 aa, synthetic construct
QVQLVESGGGLVQAGGSLRLSCAASGRTFSSYAMGWFRQAPGKERELVATISWSGGSTYYADSVKGRFTISRDNAKNTGFLQMNSLKPEDTAVYYCAADFASTVGTPLTRPAYWGQGTQVTVSS

SEQ ID NO: 29 - protein, 123 aa, synthetic construct
QVKLEESGGGAVQPGGSLRLSCTASGQTFSNYIISWFRQAPGKEREFVVGISKSGGRTYYADSAKGRFTISRDNAKNTVYLQMSSLKPEDTAVYYCAADGLVLTAAAAEYDYWGQGTQVTVSS

SEQ ID NO: 30 - protein, 124 aa, synthetic construct
QVQLVESGGGLVQAGGSLRLSCAVSGRTVSRNVMGWFRQAPGKEREFVAGIGFSGGSTYYADSVKGRFTISRDNAKNTVYLEMNRLQPEDTAVYYCAAPSLPLLTSDLHDYDYWGQGTQVTVSS

SEQ ID NO: 31 - protein, 126 aa, synthetic construct
QVQLVESGGGLVQAGGPLRLSCAASGRTFSSYAMGWFRQAPGKEREFVAAISWSGDSTYYADSVKGRFTLSRDNAKNTVYLQMNSLKPEDTAVYYCAVPSSGRGTYYYTLSAYEYWGQGTQVTVFS

SEQ ID NO: 32 - protein, 119 aa, synthetic construct
QLQLVESGGGLVQAGGSLRLSCTASGRTFRNFGMGWFRQAPGKERKLVASISYVGGNTDYADSVKGRFTISMDNAKNTVVLQMNSLKPEDTAMYYCAARNPNRNEYPWWGQGTQVTVSS

SEQ ID NO: 33 - protein, 124 aa, synthetic construct
QLQLVESGGGLVQPGGSLRLSCAASGRVYNSWTMAWFRQAPGKEREFVGAFSLLDSGTRYADSLKDRVAISRDDAANTQWLQLSALKPEDTAVYYCAAKSGTIRATSEGQYNYWGQGIQVTVSS

SEQ ID NO: 34 - protein, 121 aa, synthetic construct
EVQLVESGGGLVQAGGSLTLSCGRTTSIWGMGWFRQGRGKEREFVAAITPSGSITFYSDHVKGRFTVSRDNALNTVYLQMNSLKPEDTAVYYCARRGSSGSYYWAGSYEAWGQGTQVTVSS

SEQ ID NO: 35 - protein, 7 aa, synthetic construct
GIHMALM

SEQ ID NO: 36 - protein, 7 aa, synthetic construct
GAIIAMK

SEQ ID NO: 37 - protein, 7 aa, synthetic construct
GLLVSDW

SEQ ID NO: 38 - protein, 7 aa, synthetic construct
VVPRPSF

SEQ ID NO: 39 - protein, 7 aa, synthetic construct
VVAFGIS

SEQ ID NO: 40 - protein, 7 aa, synthetic construct
VIFNHVP

SEQ ID NO: 41 - protein, 7 aa, synthetic construct
HSGPECG

SEQ ID NO: 42 - protein, 7 aa, synthetic construct
HQPQGVW

SEQ ID NO: 43 - protein, 7 aa, synthetic construct
YPTWAYR

SEQ ID NO: 44 - protein, 7 aa, synthetic construct
FTFQGMD

SEQ ID NO: 45 - protein, 7 aa, synthetic construct
SLHRIPS

SEQ ID NO: 46 - protein, 7 aa, synthetic construct
LTGYGMS

SEQ ID NO: 47 - protein, 7 aa, synthetic construct
TSSVIQL

SEQ ID NO: 48 - protein, 7 aa, synthetic construct
AVVSFYP

SEQ ID NO: 49 - protein, 117 aa, synthetic construct
AVQLVESGGGLVQPGGSLRLSCAASGFTFTNYAMRWYRQAPGKERELVAAIPTSGGYTYTADSVKGRFTISRDNAKNTVYLQMNSLKPEDTAVYYCYGLRTLSDYYWGQGTQVTVSS

SEQ ID NO: 50 - protein, 121 aa, synthetic construct
DVQLVESGGGWVQPGGSLRLSCAASGFTFTSYALSWVRQAPGKGLEWVSLADARGTSTTYADSVKGRFTISKDNPRNTLYLLMNSLKPEDTAVYYCTKGFDGNWYPDYDYWGQGTQVTVSS

SEQ ID NO: 51 - protein, 119 aa, synthetic construct
QLQLVESGGGLVQPGGSLRLSCTASGFTFSTAGTSWYRQSPGKARKLVAELSRIGGIANYADSVKGRFTISRDNTRNTVYLQMNSLKPEDTAVYYCKVVTNRNYGFTYWGQGTQVTVSS

SEQ ID NO: 52 - protein, 126 aa, synthetic construct
AVQLVESGGGLVQPGGSLRLSCIISGFTSVSYSIVWFRQAPGKGREGVSCIGSSGGSTDYIDSVKGRFTISRDSAKNTVYLQMDCLKPEDTAVYYCAALRDVYRLCVIDYMNNDYWGQGTQVTVSS

SEQ ID NO: 53 - protein, 114 aa, synthetic construct
QVQLVESGGGLVQPGGSLRLSCAASGFTFSRKGMSWYRQVPGKEREWVARIYSTGEKTDYADSVKGRFTISRDNAKNMVYLDMNSLKPEDTAVYYCKAWGYEYWNQGTQVTVSS

SEQ ID NO: 54 - protein, 115 aa, synthetic construct
QVQLVESGGGLVQPGGSLRLSCVASGFTFSFYTMEWYRQAAGKQRELVAGLTASGTTHYADSAKGRFTISRDNAWNTVFLQMNGLKPEDTAVYYCNAYSGYRDYWGQGTQVTVSL

SEQ ID NO: 55 - protein, 117 aa, synthetic construct
QVQLVESGGGLVQPGGSLRLSCVGSGFTFSQYAMRWYRQVPGKARELVAAISRSGDFMYYADSVKGRFTISRDNVKSAVILQMNSLKPEDTAVYYCYGQATISDTLWGQGTQVTVSS

SEQ ID NO: 56 - protein, 116 aa, synthetic construct
DVQLVESGGGLVQPGGSLRLSCAASGFTFSLYYMSWYRQVPGKERELVATITSGTHATTYADSVKGRFTISRDNAKNTVSLQMNSLKREDTGVYYCKRTNAGQDPWGQGTQVTVSS

SEQ ID NO: 57 - protein, 115 aa, synthetic construct
QLQLVESGGGLVQPGGSLRLSCEASGFTFKYYTMSWYRQAPGKERELVATITNGDRTNYADSVKGRFTISRDNAKNTLYLQMNSLKPYDTAVYYCNRHLPLLQIWGQGTQVTVSS

SEQ ID NO: 58 - protein, 14 aa, synthetic construct
VAQYKALPVVLENA

SEQ ID NO: 59 - protein, 14 aa, synthetic construct
FAVANGNELLLDLS

SEQ ID NO: 60 - protein, 13 aa, synthetic construct
AKMTEEDKENALS

SEQ ID NO: 61 - protein, 14 aa, synthetic construct
ENARILKNCVDAKM

SEQ ID NO: 62 - protein, 115 aa, synthetic construct
EVQLVESGGGLVQPGGSLRLSCAASGFTFSSYAMSWVRQAPGKGLEWVSAISGRGYNADYADSVKGRFTISRDNSKNTLYLQMNSLRAEDTAVYYCAKLEYFDYWGQGTLVTVSS

SEQ ID NO: 63 - protein, 107 aa, synthetic construct
DIQMTQSPSTLSASVGDRVTITCRASQSISSWLAWYQQKPGKAPKLLIYKASSLESGVPSRFSGSGSGTDFTLTISSLRPEDFATYYCQQYNSYPLTFGGGTKVEIK

SEQ ID NO: 64 - protein, 120 aa, synthetic construct
EVQLVESGGGLAQPGGSLRLSCAASGFTFNNYAMTWVRQAPGKGLDWVSAISDSGRSTFSADSVKGRFTISRDNSKNTLYLQMDSLRAEDTALYYCAKHRNWNYPVFDYWGQGTLVTVSS

SEQ ID NO: 65 - protein, 107 aa, synthetic construct
DIQLTQSPSFLSASVGDRVTITCWASQGISSYLAWYQQKPGKAPKLLIYSASTLQSGVPSRFSGSGSGTEFTLTISSLQPEDFATYYCQQLNSYPFTFGPGTKVDIK

SEQ ID NO: 66 - protein, 119 aa, synthetic construct
EVQLVESGGGLVKPGGSLRLSCAASGFTFRNYNINWVRQAPGKGLEWVSLISGSSSYIYYADSVKGRFTVSRDNAKNSLYLQMNSLRAEDTAVYYCARRTLSYYVMDVWGQGTTVTVSS

SEQ ID NO: 67 - protein, 112 aa, synthetic construct
QVQLVESGGGLVQPGGSLRLSCAASGFTFRLAAMGWYRQAPEKEREWVASITGPGTDTNYADSVKGRFTVSRDNAKNTVYLQMNSLKPEDTAVYYCRGMGYWGKGTLVTVSS

SEQ ID NO: 68 - protein, 119 aa, synthetic construct
AVQLVDSGGGLVQAGGSLRLACAASGRTFDTYAVGWFRQAPGKERDVVASITWTSGSTWYADFVKGRFTISKDNAKNTVYLQMNNLSPEDTAVYYCGARNQIYTRWDSWGQGTQVTVSS

SEQ ID NO: 69 - protein, 122 aa, synthetic construct
QVKLEESGGGLVQAGGSLRLSCVASGRTFSRWHTGWYRQAPGREREFVATLRASGGDTYYADSVKGRFTISRDNAKNTVYLQMNSLKPEDTAVYYCNVWANWGAPPSDFSSWGQGTQVTVSS

SEQ ID NO: 70 - protein, 115 aa, synthetic construct
QLQLVESGGGLVQPGGSLRLSCAASGFTFSSYSMSWYRQAPGKERELVATIDTDGRTNYADSVKGRFTISRDNAKNSVYLQMNSLKPEDTAVYYCNRRQLGVDYWGQGTQVTVSS

SEQ ID NO: 71 - protein, 121 aa, synthetic construct
QVQLVESGGGLVQPGGSLRLSCAASGFTFSNYGMSWVRQAPGKGLEWVSDVNSSGSRRFYVDSVKGRFTISRDNAKNTVFLQMNSLKPEDSAVYYCVKLAEAGTLIHVGSWGQGTRVTVSS

SEQ ID NO: 72 - protein, 112 aa, synthetic construct
QLQLVESGGGLVQPGGSLRLSCVGSGFTFSLASMGWYRQAPGKEREWVASISSLDASTNYADSVKGRFTISRDNAKRMVYLQMNNLKSEDTAVYYCKAMNYWGKGTQVTVSS

SEQ ID NO: 73 - protein, 123 aa, synthetic construct
QLQLVESGGGLVQPGGSLRLSCAASRSIFSSYVMAWYRRAPGKKRELVASIANVGSNTDYASFAKGRFTISRDDDKSRDNDKITVYLQMNSLNPEDTAVYYCNAWLGAGSDYWGQGTQVTVSS